(19) United States
(12) Patent Application Publication
(10) Pub. No.: US 2026/0325465 A1
(43) Pub. Date: Oct. 22, 2026

(54) SHP-1 INHIBITORS FOR TREATING CANCER

(71) Applicant: MDX MANAGEMENT LLC, La Jolla, CA (US)

(72) Inventors: Yuan LIU, Decatur, GA (US); Lei SHI, Decatur, GA (US); Zhen BIAN, Tucker, GA (US); Harry STYLLI, La Jolla, CA (US)

(21) Appl. No.: 19/109,755

(22) PCT Filed: Sep. 7, 2023
(86) PCT No.: PCT/US2023/073674

§ 371 Date: Mar. 7, 2025

Related U.S. Application Data
(60) Provisional application No. 63/404,392, filed on Sep. 7, 2022.
(60) Provisional application No. 63/491,008, filed on Mar. 17, 2023.

(51) Int. Cl. A61K 31/122; A61K 31/355; A61K 31/52; A61K 38/19; A61K 38/20; A61K 39/00; A61P 35/00; C07K 16/28

(52) U.S. Cl. CPC A61K 31/122; A61K 31/355; A61K 31/52; A61K 38/191; A61K 38/2006; A61K 38/204; A61P 35/00; C07K 16/2875; A61K 2039/505

(57) ABSTRACT

The present application provides methods of treating a cancer in an individual that involves administering to the individual a SHP-1 inhibitor (such as TPI-1 or an analog or a derivative thereof) and a pro-inflammatory agent (such as a TLR agonist, a radiation therapy or an immune checkpoint inhibitor). In some cases, the methods involves administering a SHP-1 inhibitor when the individual is under an inflammation reaction.

### CROSS-REFERENCE TO RELATED APPLICATIONS

This application claims the benefit of, and priority to, U.S. Provisional Application 63/404,392, filed on Sep. 7, 2022, and U.S. Provisional Application 63/491,008, filed on Mar. 17, 2023, the contents of each of which are hereby incorporated herein by reference in their entirety.

### FIELD OF THE INVENTION

The present invention relates to compositions and methods for treating cancer involving administering a SHP-1 inhibitor and optionally a pro-inflammatory agent.

### BACKGROUND OF THE INVENTION

In cancers such as solid tumors, intratumoral myeloid leukocytes, including macrophages (i.e., tumor-associated macrophage or TAM) and myeloid-derived suppressive cells (MDSC), play critical roles in controlling the tumor microenvironment (TME) immunosuppression that supports tumor growth and also confers tumor resistance to immunotherapeutic treatments. One important mechanism by which intratumoral myeloid leukocytes adapt an immunosuppressive phenotype and strengthen their immunosuppressive capacity following tumor therapies is through their cell surface inhibitory receptors (iRs)-mediated multi-pathways of negative regulation. Phosphorylation of iRs in their cytoplasmic domain immunoreceptor tyrosine-based inhibitory motifs (ITIMs) under tumor therapies leads to activation of SHP-1, the central signal modulator, which mediates dephosphorylation and hence deactivation of a number of signal transduction molecules, resulting in diminishment of therapeutics-induced anticancer proinflammatory responses (FIG. 1). In solid tumors, essential cell surface iRs, such as SIRPα, Siglecs, LilRBs, PirB, LAIR1, lectin receptors, SLAM family receptors, etc (1, 2), which also show increased expression in the TME with tumor progression to advanced stages, conduce their regulations via activation of SHP-1, which then mediate downstream inhibition.

Given these inhibitory mechanisms elucidated within previous years, pipelines of therapeutic developments aiming to blockade iRs (e.g., anti-LilRB1/2 and anti-SIRPα) and their ligands (e.g., anti-CD47) are being undertaken (3-5). However, these efforts of targeting each iR or its ligand singularly, but not all inhibitory pathways at once, achieve weak-to-partial efficacies in controlling solid tumors.

The disclosures of all publications, patents, patent applications and published patent applications referred to herein are hereby incorporated herein by reference in their entirety.

### BRIEF SUMMARY OF THE INVENTION

The present application in one aspect provides a method of treating a cancer in an individual, comprising administering to the individual a) a SHP-1 inhibitor, and b) a pro-inflammatory agent, wherein the method comprises administering the SHP-1 inhibitor to the individual intermittently. In some embodiments, the method comprises systemically or locally (e.g., intratumorally) administering the SHP-1 inhibitor. In some embodiments, the pro-inflammatory agent comprises an agent selected from the group consisting of a TLR agonist, a STING activator, a radiation therapy, a PAMP/DAMP molecule, a checkpoint inhibitor, a pro-inflammatory cytokine, a pro-inflammatory cell, a cell, a cancer vaccine, a chemotherapeutic agent, a bacteria component, a cancer vaccine, an oncolytic virus, a sound treatment, a magnetic therapy, an electrical treatment, and an electrostatic treatment.

The present application in another aspect provides a method of treating a cancer in an individual, comprising administering to the individual a) a SHP-1 inhibitor, and b) a pro-inflammatory agent, wherein the method comprises systemically administering the SHP-1 inhibitor. In some embodiments, the method comprises administering the SHP-1 inhibitor to the individual intermittently. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, an oncolytic virus, a sound treatment, a magnetic therapy, an electrical treatment, and an electrostatic treatment.

The present application in another aspect provides a method of treating a cancer in an individual, comprising administering to the individual a) a SHP-1 inhibitor, and b) a pro-inflammatory agent, and wherein the pro-inflammatory agent comprises an agent selected from the group consisting of a TLR agonist, a STING activator, a PAMP/DAMP activator, a chemotherapy, a pro-inflammatory cytokine, a cancer vaccine, a bacteria component, a sound treatment, a magnetic therapy, an electrical treatment, and an electrostatic treatment. In some embodiments, the method comprises administering the SHP-1 inhibitor to the individual intermittently. In some embodiments, the method comprises systemically administering the SHP-1 inhibitor.

The present application in another aspect provides a method of treating a cancer in an individual, comprising administering to the individual a SHP-1 inhibitor, wherein the individual is under an inflammation reaction or has an ongoing infection. In some embodiments, the method comprises administering the SHP-1 inhibitor to the individual intermittently. In some embodiments, the method comprises systemically administering the SHP-1 inhibitor. In some embodiments, the method further comprises immune cells.

In some embodiments according to any one of the methods described above, the method comprises administering the SHP-1 inhibitor to the individual at an interval of no more than once every three days for at least twice.

In some embodiments according to any one of the methods described above, the method comprises administering the SHP-1 inhibitor to the individual for at least two cycles, wherein each cycle has about three to about twenty days.

In some embodiments according to any of the methods described above, the SHP-1 inhibitor has a half-life of no more than about 5 days, optionally the SHP-1 inhibitor has a half-life of no more than about 3 days.

In some embodiments according to any of the methods described above, the SHP-1 inhibitor is effective in inhibiting more than 50% of the SHP-1 activity for no more than about 5 days, optionally wherein the SHP-1 inhibitor is effective in inhibiting more than 50% of the SHP-1 activity for no more than about 3 days.

In some embodiments according to any of the methods described above, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1), or a protein agent that contains a SH2 domain (by competing for binding to ITIM motif so to inhibit SHP-1 activation), a tyrosine kinase inhibitor that inhibit ITIM phosphorylation thereby inhibit SHP-1 activation. In some embodiments, the SHP-1 inhibitor is selected from the group consisting of TPI-1 or an analog or a derivative thereof, vitamin E derivative, phomoxanthone A (PXA), and a PKCθ activator. In some embodiments, the SHP-1 inhibitor comprises TPI-1.

In some embodiments according to any of the methods described above, the SHP-1 inhibitor is administered at least three times. In some embodiments according to any one of the methods described above, the method comprises administrating the SHP-1 inhibitor systemically and locally, optionally wherein the method comprises intratumorally administering the SHP-1 inhibitor.

In some embodiments according to any of the methods described above, the systemic administration of SHP-1 comprises oral administration, intravenous administration, subcutaneous administration, and/or intraperitoneal administration.

In some embodiments according to any of the methods described above, the pro-inflammatory agent and the SHP-1 inhibitor are administered within about 24 hours (e.g., within about 16 hours, 8 hours, 4 hours, 2 hours, 1 hour, or 0.5 hour) of each other.

In some embodiments according to any of the methods described above, the method comprises intratumorally administering the pro-inflammatory agent.

In some embodiments according to any of the methods described above, the method comprises administering the pro-inflammatory agent to a site that is different from the site of the cancer to be treated.

In some embodiments according to any of the methods described above, the pro-inflammatory agent comprises a TLR agonist. In some embodiments, the TLR agonist activates a TLR on a macrophage. In some embodiments, the TLR comprises TLR2, TLR3, TLR4, TLR5, TLR6, TLR7, TLR8, and/or TLR9. In some embodiments, the TLR agonist comprises CpG, polyL:C and/or R848, flagellin (TLR5), zymosan (TLR2/4), radiation therapy produced DAMP such as HMGB1 (TLR2/4), DNA and RNA molecules (TLR3/7/8/9), etc.

In some embodiments according to any of the methods described above, the pro-inflammatory agent comprises a bacteria component, optionally the bacteria component comprises lipopolysaccharide (LPS).

In some embodiments according to any of the methods described above, the pro-inflammatory agent comprises a STING activator. In some embodiments, the STING activator comprises 2′3′-cGAMP.

In some embodiments according to any of the methods described above, the pro-inflammatory agent comprises a chemotherapeutic agent. In some embodiments, the chemotherapy comprises azathioprine (AZA).

In some embodiments according to any of the methods described above, the pro-inflammatory agent comprises a pro-inflammatory cytokine. In some embodiments, the pro-inflammatory cytokine comprises IL-1 family cytokines (e.g., IL-1b, IL-18), IL-6, IL-17, TNF family cytokines (e.g., TNFα), and their combination with type I and type II interferons (IFNα, IFNβ and IFNγ).

In some embodiments according to any of the methods described above that applies, the pro-inflammatory agent comprises a radiation therapy. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is non-ablative, insufficient to eliminate tumor (kill all tumor cells).

In some embodiments according to any of the methods described above, the pro-inflammatory agent comprises a checkpoint inhibitor. In some embodiments, the checkpoint inhibitor comprises an anti-PD-L1 antibody, an anti-PD-1 antibody or an anti-CLTA4 antibody.

In some embodiments according to any of the methods described above, the pro-inflammatory agent is administered intermittently.

In some embodiments according to any of the methods described above, the pro-inflammatory agent and the SHP-1 inhibitor are administered simultaneously or concurrently.

In some embodiments according to any of the methods described above, the pro-inflammatory agent comprises immune cells. In some embodiments, the immune cells are derived from the same individual. In some embodiments, the immune cells comprise or are macrophages, optionally wherein the macrophages have a proinflammatory (M1) phenotype. In some embodiments, the immune cells are derived from monocytes. In some embodiments, the immune cells express a high level of MHC-I, MHC-II, CD80 and/or CD86. In some embodiments, the immune cells express one or more pro-inflammatory cytokines, optionally wherein the one or more pro-inflammatory cytokines comprise TNFα and/or IL-12. In some embodiments, the immune cells do not express a significant level of TGFβ and/or IL-10. In some embodiments, the immune cells comprise T cells. In some embodiments, the immune cells are engineered to express a chimeric antigen receptor, optionally wherein the chimeric antigen receptor specifically binds to a tumor antigen. In some embodiments, the macrophages are engineered to be deficient in SHP-1 expression and/or activation. In some embodiments, the SHP-1 inhibitor and the immune cells are administered within 24 hours of each other, optionally wherein the SHP-1 inhibitor and the immune cells are administered within 4 hours of each other. In some embodiments, the immune cells are administered simultaneously or concurrently with the SHP-1 inhibitor.

In some embodiments according to any of the methods described above, the method further comprises administering to the individual an agent that reduces systemic inflammation and/or reduces inflammatory cytokine cascade or cytokine storm, including, but not limited to, an anti-TNFα antibody and an anti-IL6 antibody. In some embodiments, the agent that reduces systemic inflammation and/or reduces inflammatory cytokine cascade or cytokine storm is administered simultaneously with the tyrosine kinase inhibitor. In some embodiments, the agent that reduces systemic inflammation and/or reduces inflammatory cytokine cascade or cytokine storm is administered sequentially (e.g., prior to or after) with the tyrosine kinase inhibitor. In some embodiments, the administration of the agent that reduces systemic inflammation and/or reduces inflammatory cytokine cascade or cytokine storm follows the same dosing schedule as the tyrosine kinase inhibitor.

In some embodiments according to any of the methods described above, the cancer is a solid tumor.

In some embodiments according to any of the methods described above, the cancer is a hematological cancer.

In some embodiments according to any of the methods described above, the cancer is a late stage cancer.

In some embodiments according to any of the methods described above, the cancer is resistant or refractory to a radiation therapy, a chemotherapeutic agent, and/or a checkpoint inhibitor.

In some embodiments according to any of the methods described above, the individual is a human.

The present application in another aspect provides a composition comprising a SHP-1 inhibitor and a pro-inflammatory agent, optionally wherein the pro-inflammatory agent comprises an agent selected from the group consisting of immune cells, a TLR agonist, a STING activator, an agent used in radiation therapy, a PAMP/DAMP activator, a checkpoint inhibitor, a pro-inflammatory cytokine, a chemotherapeutic agent, a bacteria component, a cancer vaccine, an oncolytic virus, and an agent used in sound treatment, a magnetic therapy, an electrical treatment or an electrostatic treatment.

### BRIEF DESCRIPTION OF THE DRAWINGS

FIG. 1 depicts that SHP-1 serves as the “master” signaling mediator downstream of multi-inhibitory receptors on myeloid leukocytes in the tumor microenvironment (TME). The activity of SHP-1 diminishes RT- and immunotherapies-induced proinflammatory pathways and anti-cancer efficacy, and sustains myeloid leukocyte immunosuppressive phenotype. Our approach of SHP-1 inhibition as an anti-cancer strategy (red). Companies and approaches that aim to deplete or blockade individual cell surface inhibitory receptors including SIRPα (SIRPαnt Immunotherapeutics, anti-CD47 Gilead) and anti-SIRPα (Biosion) approaches), Siglec (NextCure), LilRB (Next-IO), SLAMF (BMS), etc. are partially listed.

FIGS. 2A-2G depict that the inhibition of SHP-1 enhances macrophage proinflammatory response, antigen presentation and phagocytosis in a tumor milieu. FIG. 2A and FIG. 2B depict that SHP-1 activity in macrophages is triggered by proinflammatory stimulation and the extracellular cancer cell ligation (“touching”) of macrophages (green bar in FIG. 2A). The SHP-1 activity is inhibited by a catalytic domain covalent inhibitor TPI-1 (1 μg/ml, FIG. 2A) in a dose-dependent manner (FIG. 2B). The SHP-2 inhibitor SHP099 (1 μg/ml) was used in assays. FIG. 2C depicts the inhibition of SHP-1 by TPI-1 dose-dependently recovers IFNγ/LPS-induced activation of STAT1 (p-STAT1) and Erk1/2 (p-Erk1/2) de-activated by SHP-1 (red line). FIG. 2D and FIG. 2E depict that the inhibition of SHP-1 in macrophages by TPI-1 enhances production of proinflammatory cytokines (FIG. 2D) and expression of immunogenic antigen presentation machinery (FIG. 2E) by IFNγ/LPS in a tumor milieu. FIG. 2F and FIG. 2G depict that the inhibition of SHP-1 promotes proinflammatory-activated macrophages for phagocytosis of cancer cells. FIG. 2F shows human monocytes-derived macrophages phagocytosing THP-1 leukemia cells and HT29 colon cancer cells. FIG. 2G shows murine bone marrow-derived macrophages phagocytosing syngeneic cancer cells EL4 (T lymphoma), B16 (melanoma), MC38 (colorectal cancer), Pan01 and KPC (both pancreatic adenocarcinoma), and LLC (lung cancer). The SHP-1 and SHP-2 inhibitors TPI-1 and SHP099, respectively, was used at 1 μg/ml in assays.

FIGS. 3A-3E depict modulation of SHP-1 activity in proinflammatory macrophages in cancer. Macrophages co-cultured with cancer cells were stimulated with IFNγ/LPS or TLR agonists (αTLR) comprising CpG, polycytidylic acid (i.e., PolyI:C) and R848 (each 0.4 μg/ml) for 30 min (37 °C) in the presence or the absence of different SHP-1 or SHP-2 inhibitor or activator, followed by cell lysis and assays of PTP activity. FIG. 3A depicts SHP-1 activity regulation mechanisms. FIG. 3B and FIG. 3C depict PTP activity in macrophages induced by IFNγ/LPS (FIG. 3B) or TLR agonists (FIG. 3C) in the presence of cancer cell ligation is largely (>70%) diminished by inhibition of SHP-1 (TPI-1 and PTP-I) or pan-PTP inhibitors PTP-III and pervanadate, but only weakly reduced (<10%) by inhibition of SHP-2 (SHP099 and PHPS1). FIG. 3D depicts that Vitamin E derivatives and Phomoxanthone A (PXA) dose-dependently, moderately inhibited SHP-1 activity in IFNγ/LPS-stimulated macrophages around cancer cells. FIG. 3E depicts that PKCθ negatively regulates SHP-1 activity at a moderate level. Inhibition of PKCθ by PKCθ inhibitor I and VTX27 increased SHP-1 activity by IFNγ/LPS and cancer cell ligation. Conversely, activation of PKCθ by PMA reduced SHP-1 activity.

FIGS. 4A and 4B depict that pulse inhibition of SHP-1 transiently enhances proinflammatory signal transduction in macrophages. FIG. 4A: murine macrophages (bone marrow derived macrophages, or “BMDM”) were treated with TPI-1 for 15 min followed by wash to complete removal of TPI-1. At different time points after the TPI-1 treatment, macrophages were stimulated with IFNγ/LPS in the presence of alive cancer cells (1:1 ratio to BMDM) for 20 min prior to cell lysis and assaying SHP-1 activity using pNpp and WB detecting total and phosphorylated STAT1 and Erk1/2. FIG. 4B: the same setting as in FIG. 4A, except that after TPI-1 treatment, TPI-1 in medium was partially removed (50%) or without removal, followed by macrophage stimulation with IFNγ/LPS at different time points.

FIGS. 5A-5C depict signaling mechanisms when SHP-1 is targeted in solid tumor. FIG. 5A depicts a summary of the SHP-1 mechanism in solid tumor. SHP-1 remains inactive/low activity in solid tumors until therapeutic treatments. SHP-1 is activated following a tumor-protective feedback loop: proinflammatory signals→tyrosine kinase (TK)→ITIMs phosphorylation→SHP-1 activation. SHP-1 activity then inhibits proinflammatory signaling and confers tumor resistance to therapies. TPI-1 inhibits SHP-1 activity and restores proinflammatory signal transduction leading to anti-tumor innate and adaptive immunity.

FIG. 5B depicts an example seen in SIRPα. Proinflammatory signal-induced Src family TK(s) mediate phosphorylation in the cytoplastic domain ITIMs that dock SHP-1 leading to SHP-1 activation. FIG. 5C depicts that proinflammatory stimulations-induced phosphorylation in iR ITIMs leads to exclusively binding of SHP-1 to pITIMs, leading to SHP-1 activation; whereas immunosuppressive signals by IL-4, IL-10 and TGFβ induce ITIMs phosphorylation leads to binding of SHP-1. Examples shown macrophage iRs LilRB and SIRPα.

FIGS. 6A-6D depict that the inhibition of SHP-1 promotes anti-tumor effects or pro-tumor effects under different circumstances. FIG. 6A depicts that intratumoral macrophages upregulate expression of iRs (Pir-B, Siglec E, F and G, and SIRPα) along with tumor progression to late-stage large sizes. KPC pancreatic tumors of small sizes (<150 mm³) and those grown to larger sizes (>800 mm³) were dissociated to single cells, followed by flow cytometry analyses for cell surface protein expression on macrophages (gated F4/80+). FIG. 6B depicts that the inhibition of SHP-1 alone promotes TME immunosuppression. Excised MC38 solid tumor cubes were treated with SHP-1 inhibitor TPI-1 (100 nM) or vehicle (DMSO) in a cell culture setting. After 24 h, tumor secreting cytokines into the culture medium were assayed by ELISA. As shown, TPI-1 treatment increased tumor production of IL-6 and IL-10. FIG. 6C depicts that the inhibition of SHP-1 by TPI-1 and PTP-1 dose-dependently augmented macrophage production of IL-10 and TGFβ under immunosuppressive stimulation by IL-4/13 or IL-10 in the presence of cancer cell ligation. FIG. 6D with the same experimental setting demonstrated that TPI-1 and PTP-1 dose-dependently augmented macrophage proinflammatory response with increased IL-12 and TNFα induced by IFNγ and LPS.

FIGS. 7A-7F depict that the inhibition of SHP-1 unleashes proinflammatory response and antigen presentation in solid tumor upon therapies. FIG. 7A shows that intratumorally treating KPC pancreatic tumor with TLR agonists (αTLR, comprising CpG, PolyI:C and R848, each 1 μg), proinflammatory cytokines (IL-1β, IL-6, TNFα and IFNγ, each 10 ng) and the STING activator 2′3′-cGAMP (1 μg) for 30 min induced spikes of PTP activities that were abated by the SHP-1 inhibitor TPI-1. Low PTP/SHP-1 activity was found in untreated, homeostatic tumor. FIG. 7B shows that similarly, PTP/SHP-1 activities in KPC tumor were induced by a fraction of 8Gy RT treatment, a dose of chemotherapy with Azathioprine (AZA) or anti-PD-L1 antibody (αPD-L1), and these activities were abated by the simultaneous treatment with TPI-1. FIG. 7C depicts that depletion of intratumoral macrophages with clodronate liposomes eliminated SHP-1 activity induced by various treatments to tumor. FIG. 7D and FIG. 7E show that the inhibition of SHP-1 largely augmented TLR agonists- and RT-induced proinflammatory cytokines (FIG. 7D) and capacity of immunogenic antigen presentation of intratumoral macrophages (FIG. 7E). Note that treating tumors with TLR agonists or RT without SHP-1 inhibition induced minute increases in proinflammatory cytokines but significant IL-10 and TGFβ. FIG. 7F depicts that transcription profiling revealed markedly different tumor responses to TLR agonists and RT without and with intratumoral SHP-1 inhibition. Without SHP-1 inhibition, KPC tumors exhibited treatment resistance with increased immunosuppressive TGFβ signaling and MDSC infiltration, whereas treated tumors with SHP-1 inhibition led to TME reprogramming to a strong proinflammatory niche with reduced TGFβ but high expression of inflammatory cytokines, antigen presentation molecules and chemokines that attract neutrophils, NK and T cells, but not MDSC. Similar data were obtained by studying colorectal carcinoma MC38.

FIGS. 8A-8E depict that SHP-1 inhibition combined with TLR agonists (αTLR) reprograms TME of MC38 colorectal carcinoma. FIG. 8A depicts MC38 tumor treatment scheme. FIG. 8B-8C show that TME analyses demonstrate reprogramming of TME by TPI-1 combined αTLR treatment, inducing reduction of tumor cells, and increases in immune infiltration especially tumoricidal CD8 T cells, neutrophils (PMN) and NK cells, while reduction of macrophages, MDSC and Treg. FIG. 8D show marked reduction of intratumoral macrophages following TPI-1 and αTLR treatment. FIG. 8E show ex vivo treating excised MC38 tumor with TPI-1 and αTLR induced CD8 T cell expansion, suggesting that the treatment induced antigen presentation in situ.

FIGS. 9A-9C depict that the inhibition of SHP-1 by TPI-1 combined with tumor-focal RT reprogram TME of KPC pancreatic ductal adenocarcinoma towards proinflammatory cancer elimination. FIG. 9A depicts treatment scheme and TME analyses on day 5. The table depicts the percentages of various populations within total CD45+ cells. The bar graph depicts the percentages of various populations within total cells. FIG. 9A demonstrates that TPI-1 combined RT induced neutrophil (PMN) infiltration, NK cell increases and CD8 T cell expansion. FIG. 9B depicts that TPI-1 combined RT treatment induced marked expansion of CD8 T cells with significant high frequency of reactivity to tumor-specific antigen p15E. FIG. 9C depicts that intratumoral macrophages demonstrated proinflammatory phenotype and increased antigen presentation capacity following TPI-1 combined RT treatment.

FIGS. 10A-10B depict Pulse-intermittent SHP-1 inhibition (iShp-1) strategy treating metastatic solid tumor. FIG. 10A: preclinical metastatic solid tumor models are established in syngeneic WT mice by multi-location engraftments. After tumors formation, mice are treated with SHP-1 inhibitor combined proinflammatory modalities that kickstart anti-cancer immunity. Treatments are given via i.p. or s.c. to achieve systemic effects. Treatments can also be given via intratumoral injection (i.t.). FIG. 10B: treatment schemes and assessment: Pulse-intermittent schemes administrate SHP-1 inhibitor once, or consecutively twice or three times (pulse-1, -2 or -3) at the beginning of each cycle, followed by an intermittent period (2-9 variable days) before the next cycle of treatment. Combination modalities, not limited to the list, are administered simultaneously (e.g., TLR agonists, shown in the figure), or otherwise following specific dosing schedules. Throughout experiments, tumor control efficacies, adverse effects and toxicity are evaluated. The TME immunogenicity changes are also determined for mechanistic insights.

FIGS. 11A-11E depict the impact of continuous or intermittent iShp-1 treatment on the efficacy and adverse toxicity. FIG. 11A depicts the study design. Mice with KPC pancreatic cancer were either continuously treated with TPI-1 (1× per day) or in an intermittent fashion with gapping days between two treatments (intermittent). Three doses, 1, 3 and 10 mg/kg (i.p.), were tested and TLR agonists (CpG plus PolyI:C, each 10 μg, i.p., 1× every 3 day) was given to kickstart inflammatory response. FIGS. 11B and 11C shows tumor treatment efficacies. Tumor imaging (FIG. 11B) and volume change records (FIG. 11C) indicate similar efficacies of iShp-1 with continuous or intermittent schemes. FIGS. 11D and 11E shows adverse effects. Daily records of body weight, blood hemoglobin, proteinuria and serum alanine transaminase (ALT) and splenomegaly analyses at the terminal point (d11) demonstrated high risks of continuous iSHP-1, which caused anemia, kidney damage, splenomegaly and lung inflammation (not shown). However, intermittent iShp-1 demonstrated low risks of adverse effects.

FIGS. 12A-12D depict that pulse-intermittent inhibition of SHP-1 (iSHP-1) combined with TLR agonists (αTLR) and/or anti-PD-L1 checkpoint inhibitor effectively treat multi-lesion MC38 colorectal carcinoma. FIG. 12A depicts the treatment scheme. Mice with bilateral MC38 tumors (s.c.) were treated for two days with TPI-1 and various combinations via i.p. or s.c. A 5-day intermittent period was given before the 2^{nd} cycle of treatments to mice that had residue tumors. FIGS. 12B and 12C depict tumor control efficacies measured by tumor volume changes (FIG. 12B) and TME reprogramming, indicating increases in tumor killing immune populations while reduction of immunosuppression. FIG. 12D depicts acute adverse toxicity, measured by body weight, proteinuria, serum ALT level and splenomegaly.

FIGS. 13A-13E depict pulse-intermittent SHP-1 inhibition (iSHP-1) combination with RT and αPD-L1 treating pancreatic cancer and lung cancer. FIG. 13A depicts experimental schemes. On day 1, mice with bilateral KPC pancreatic cancer or LLC lung cancer were treated with a single pulse dose of TPI-1 (3 mg/kg) via i.p. to systemically inhibit SHP-1 (iSHP-1). Concomitantly, the tumor at the right flank was treated with 8Gy X-ray radiation (RT). After two days of intermittent period, mice on day 4 were treated with the 2^{nd} cycle TPI-1 (i.p.) of the same dose while RT reduced to 4Gy applied to the right flank tumor. The 3^{rd} cycle (day 7) iSHP-1 combination with RT reduced to 2Gy. Anti-PD-L1 Ab (100 μg, i.p.) was given a day following TPI-1 plus RT. FIG. 13B depicts luminescent image tracing KPC-luc and LLC-luc tumor changes post treatment. FIG. 13C depicts record of tumor volume changes and animal survival up to post-treatment 45 days. FIG. 13D and FIG. 13E show that the treatments did not cause splenomegaly, body weight loss or anemia (FIG. 13D), nor did it incur lung inflammation (FIG. 13E).

FIGS. 14A-14E depict that pulse-intermittent inhibition of SHP-1 (iSHP-1) combined with TLR agonists (αTLR) treating late-stage KPC pancreatic ductal adenocarcinoma. FIG. 14A depicts the treatment scheme. Mice with large KPC tumors were treated (i.p.) consecutively for three days with TPI-I plus TLR agonists (CpG, PolyIC and R848, each 50 μg). After the initial pulse treatment, an intermittent period of 9-day was given before the 2^{nd} cycle of treatment of two days with TPI plus TLR agonists. FIG. 14B depicts the luminescent images of mice with KPC tumor during the course of treatment. FIG. 14C depicts tumor volume changes. FIG. 14D depicts TME analyses on day 4 revealed reduction of intratumoral immunosuppressive populations including macrophages (MØ) and MDSC, and increases in tumor-killing CD8 T cells (Tc), inflammatory neutrophils (PMN) and NK cells. FIG. 14E depicts the treatment scheme incurred minor adverse effects and transient body weight loss followed by recovery.

FIG. 15 depicts proteomic analyses of protein tyrosine phosphatase expression in macrophages.

FIGS. 16A-16D depicts activation of macrophage proinflammatory response and antigen presentation in a tumor milieu by SHP-1 inhibition (iShp1) combination regimens. FIG. 16A depicts the testing system. FIG. 16B and FIG. 16C depict effects of interferons with or without TPI-1. FIG. 16D depicts effects of various agents including IL-1 family cytokines (IL-1, IL-18), TNFα and TLR ligands combined with TPI-1.

FIGS. 17A-17C depict inhibition of SHP-1 (iShp1) abrogates tumor-imposed immune suppression under pro-inflammatory challenges. FIG. 17A depicts neutrophil infiltration in different organs measured at various time point following αTLR challenge. FIG. 17B depicts neutrophil infiltration in tumor tissues in mice treated with αTLR plus TPI-1. FIG. 17C depicts the phenotype of intratumoral macrophages.

FIGS. 18A-18G show that anti-TNFα mAb curbs down systemic inflammation and reduces adverse toxicity. FIG. 18A shows the experimental design. Mice with established MC38 colorectal carcinoma (200-400 mm3) were treated with αTLR, TPI-1 and Dasatinib (s.c.), without or with additional treatment with anti-TNFα mAb or anti-IL-6 mAb (150 μg, i.p.). The treatment was repeated once (d1 and d2). Tumor volume changes were recorded, and tumor TMEs were analyzed for immune infiltrates on day 6 post treatments. FIG. 18B shows tumor volume changes following various treatments. FIG. 18C and FIG. 18D show results of TME analyses. Anti-TNFα mAb or anti-IL-6 mAb treatment did not affect αTLR/TPI-1/Dasatinib therapy-induced increases in CD8 T cells (Tc) and NK cells, as well as reduction of macrophages and MDSC in the TME. FIG. 18E shows that treating mice with anti-TNFα mAb, but not anti-IL-6 mAb, largely diminished the induction of inflammatory cytokines (TNFα, IL-6, IL-1β, IL-10, IFNα and IFNγ) associated with the αTLR/TPI-1/Dasatinib combination therapy. FIG. 18F shows that anti-TNFα treatment also markedly reduced monocyte and PMN chemokines CCL2, CCL5 and CXCL1 in circulation, while without reducing CXCL10 that is essential for T cell trafficking. FIG. 18G shows that anti-TNFα treatment protected mice from developing splenomegaly and intestinal inflammation that were commonly associated with αTLR/TPI-1/Dasatinib therapy.

FIGS. 19A-19C and 20. shows mechanism by which tumor cells inhibit macrophage proinflammatory response in TME.

FIGS. 21A-21E shows upregulation of iRs and their ligands when tumors progress to late stages.

FIGS. 22A-22E shows cancer cells- and tumor TME-produced factors (secretome) induce increases in macrophage expression of iRs.

FIGS. 23A-23B show inhibition of SHP-1 unleashes proinflammatory response in KPC tumor TME.

FIG. 24 shows that treating MC38 tumor with TLR agonist (αTLR) plus SHP-1 inhibition led to proinflammatory polarization of TME.

FIGS. 25A and 25B show the iRs→SHP-1 Inhibitory Axis. FIG. 25A shows western blot analyses of JAK-STAT, NFκB, MAPK, and PI3K-Akt signaling pathway activation and protein phosphorylation triggered by LPS (1 μg/ml) plus IFNγ (40 ng/ml) stimulation. FIG. 25B shows densitometry analyses of protein phosphorylation and hence signal transduction activation.

FIGS. 26A-26C show that Shp1^{-/-} macrophages resist cancer cells-imposed inhibition and unleash proinflammatory response under TLR and IFNγ stimulation.

FIGS. 27A-27C show that cell surface blockade of iRs or ligands as an alternative strategy to deplete the iRs→SHP-1 axis of inhibition.

### DETAILED DESCRIPTION OF THE INVENTION

The present application in one aspect provides methods of treating a cancer in an individual, comprising administering to the individual a SHP-1 inhibitor, wherein the individual a) has been subject to, is being subject to, or is about to be subject to a pro-inflammatory agent, or b) is under an inflammation reaction or has an ongoing infection. The present application in another aspect provides methods of treating a cancer in an individual, comprising administering to the individual monocytes or macrophages deficient in SHP-1 expression or activation, and wherein individual a) has been subject to, is being subject to, or is about to be subject to a pro-inflammatory agent, or b) is under an inflammation reaction or has an ongoing infection. In some embodiments, the SHP-1 inhibitor is administered systemically. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, an oncolytic virus, a radiation therapy, a sound treatment, a magnetic therapy, an electrical treatment, and an electrostatic treatment.

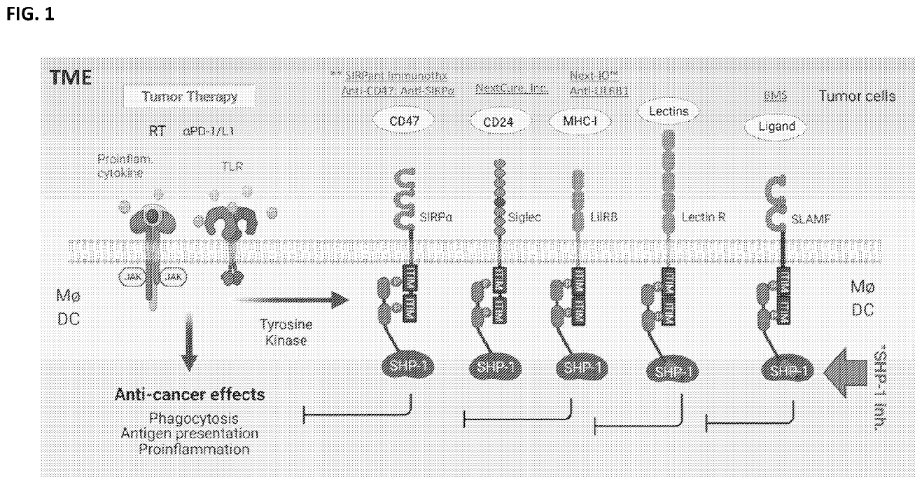

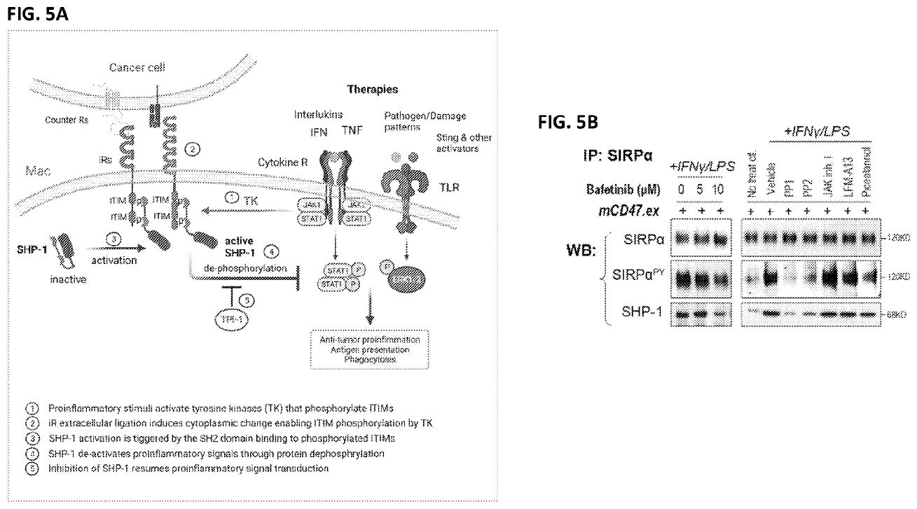

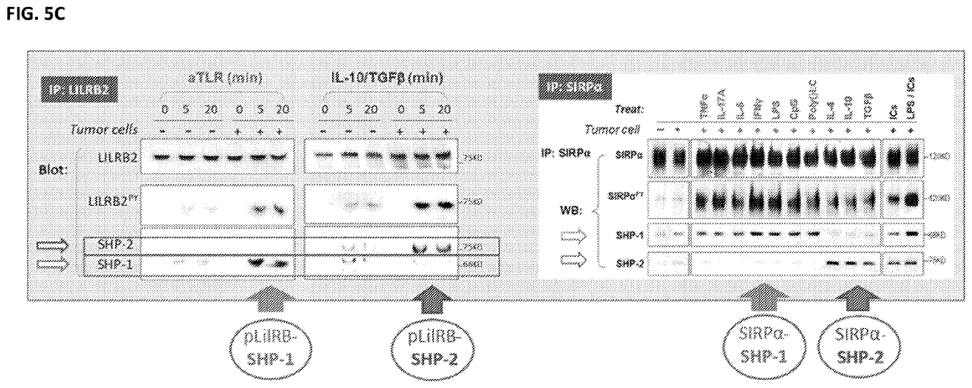

The present application is at least partly based upon a striking finding that combing a “master” inhibitory executor, SHP-1 with a pro-inflammatory treatment unleash proinflammatory signal transduction in tumor environment, especially working on tumor infiltrating macrophages, leading to drastic reprogramming of the TME and bolstering activation of innate and adaptive immune cells to promote anti-cancer immunity. Specifically, it was found that intratumoral iRs-SHP-1 mediated inhibitory regulations are particularly strong under tumor therapies, as these treatments often induce ITIMs to be hyper-phosphorylated, thereby spurring ‘hyper-activation’ of SHP-1, a feedback loop safeguarding tumors from therapeutic damage and inflammatory afront, and also eliciting wound healing response to promote tumor progression. See e.g., FIG. 1 and FIG. 5A. This finding underscores the necessity of inhibiting SHP-1 as a combination in tumor immunotherapy in order to achieve efficacies.

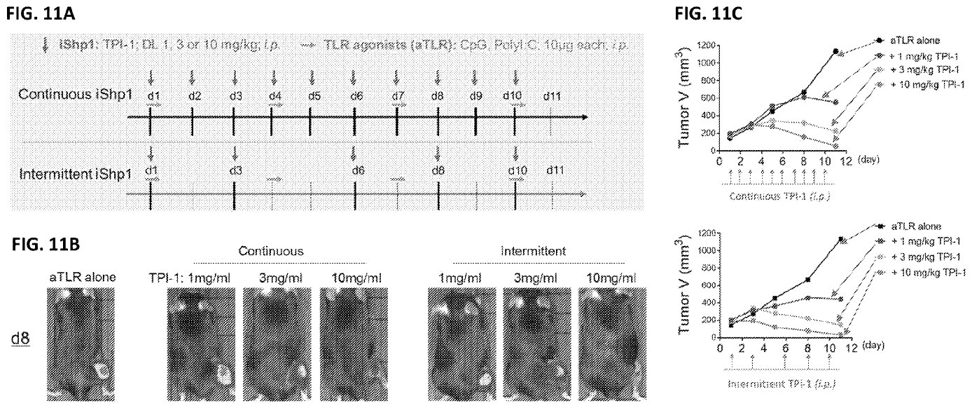

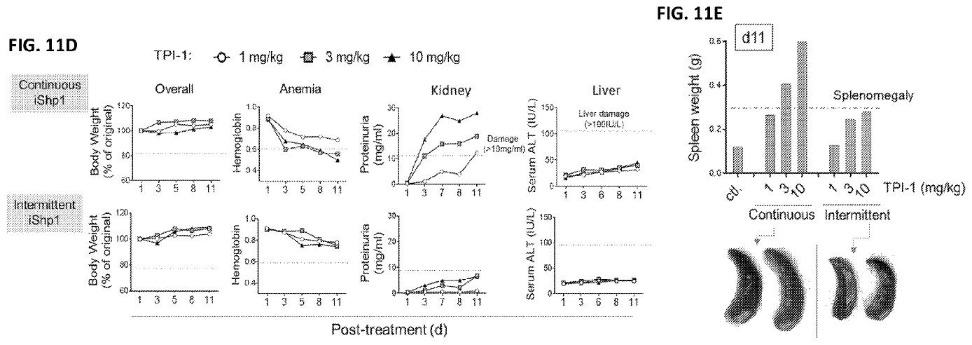

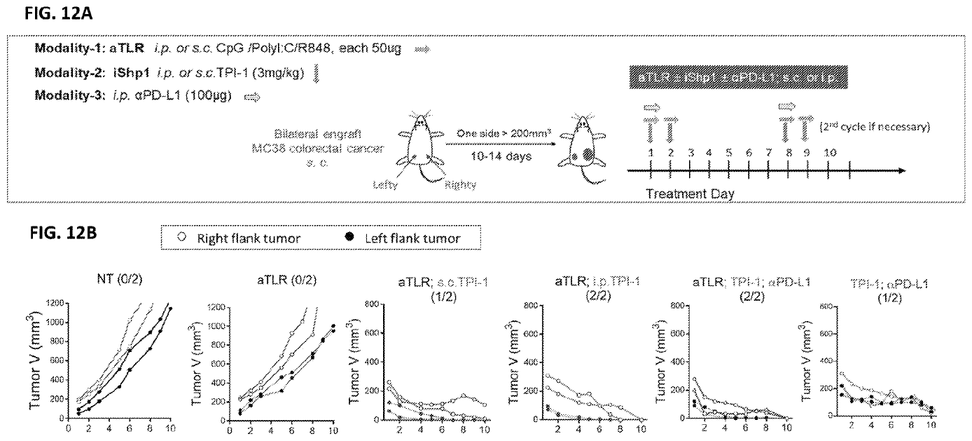

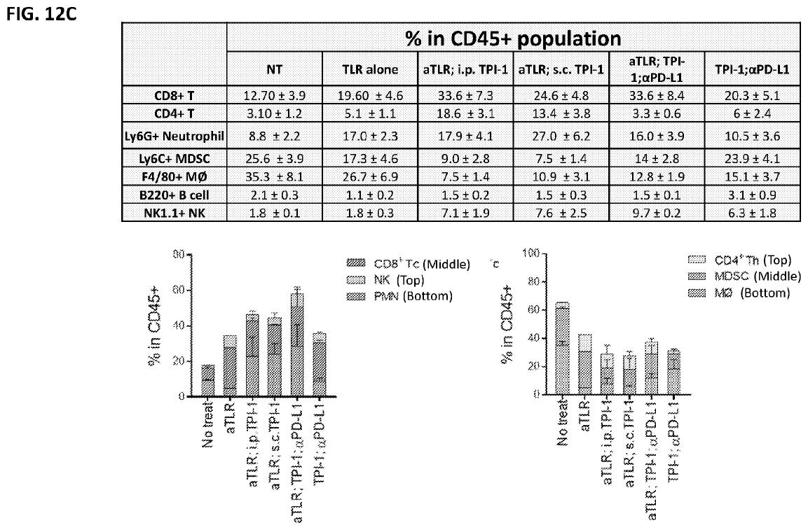

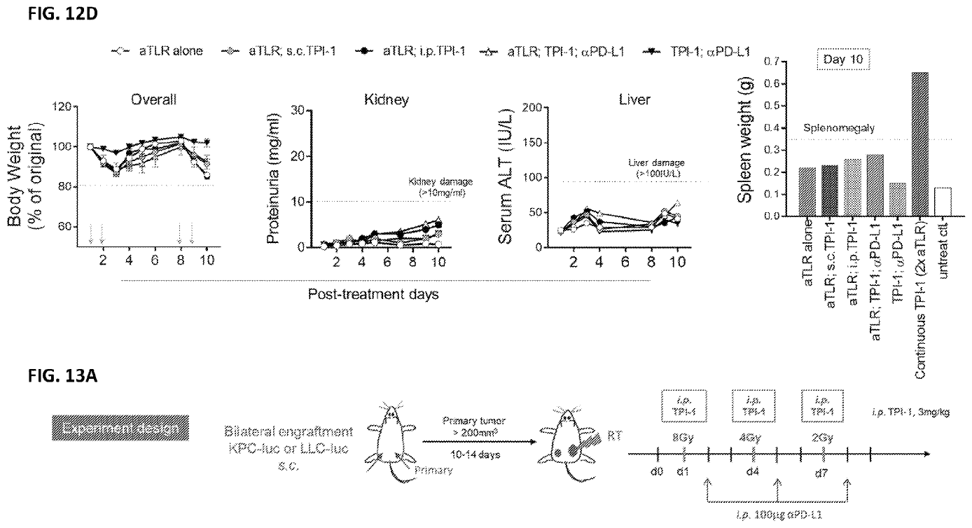

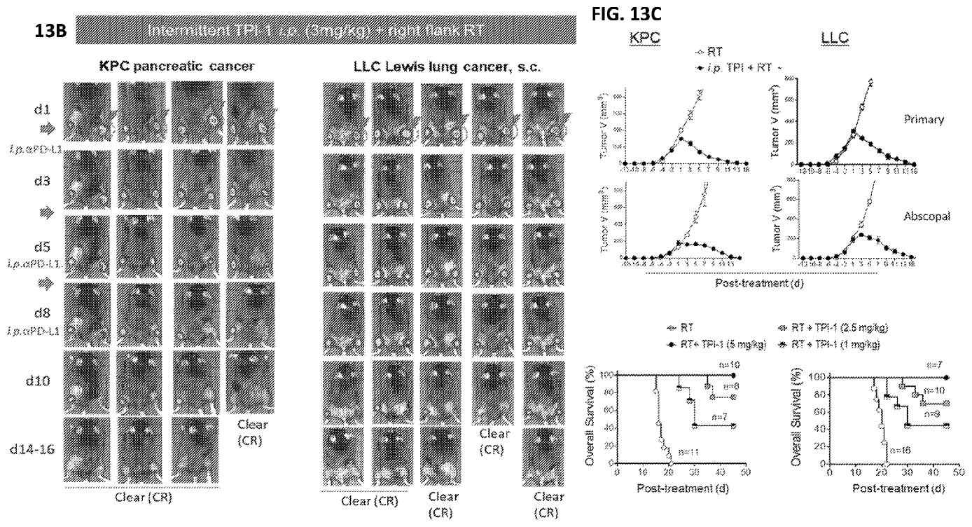

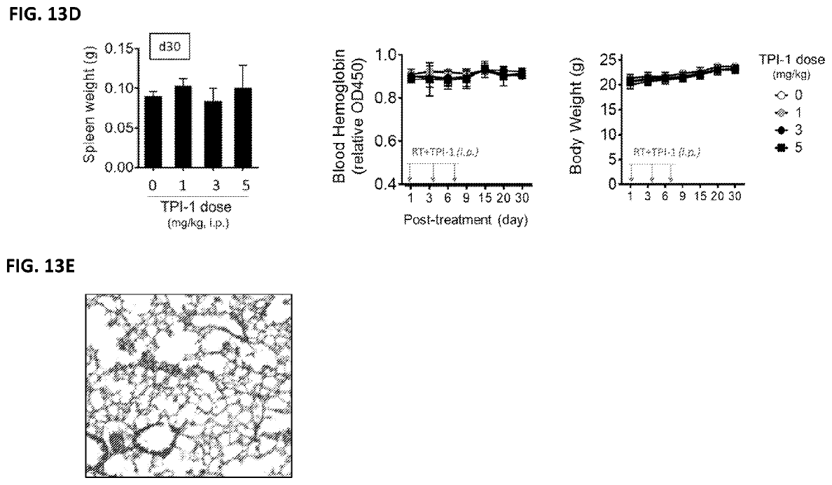

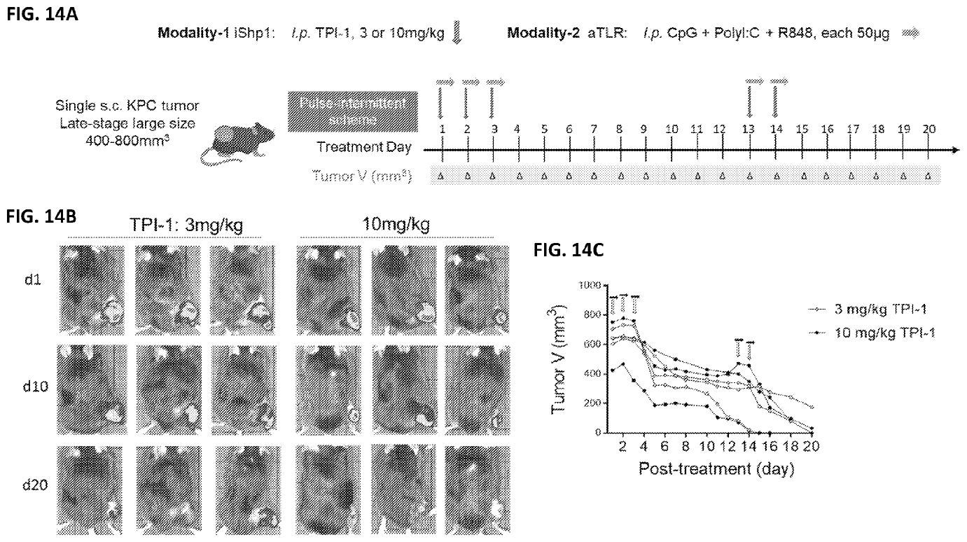

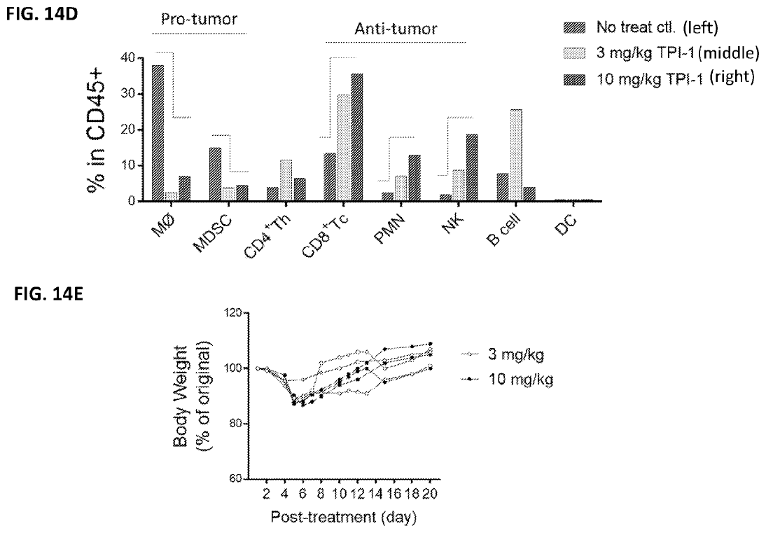

It was demonstrated that combining a SHP-1 inhibitor (e.g., TPI-1) with a pro-inflammatory agent (such as TLR agonists, pro-inflammatory cytokines, radiation therapies, checkpoint inhibitors) achieved striking effects of transforming an immunosuppressive TME into an inflammatory TME, energizing various types of immune cells (such as macrophages, T cells, and B cells), and completely depleting tumors. See e.g., FIGS. 11C and 12B. It was also demonstrated that this combination therapy achieved an abscopal effect (e.g., FIG. 13B) and it is effective in treating late stage large size tumors (e.g., FIG. 14C).

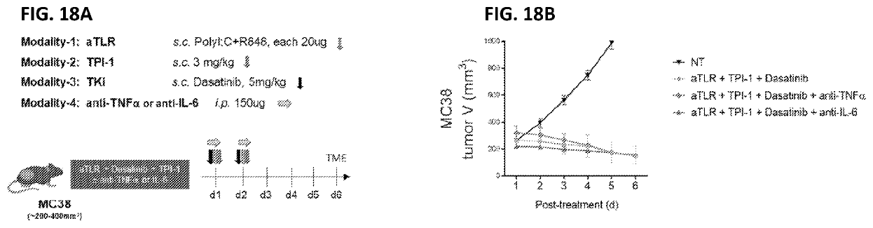

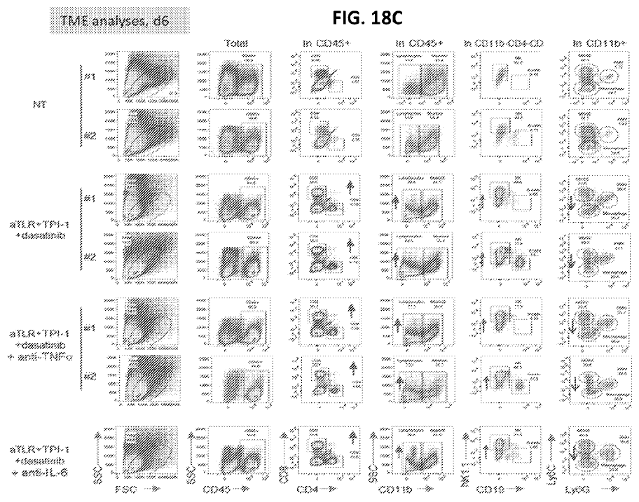

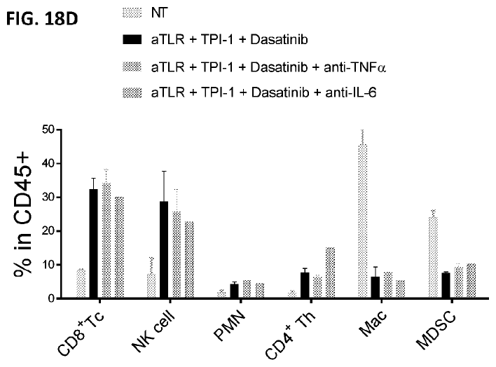

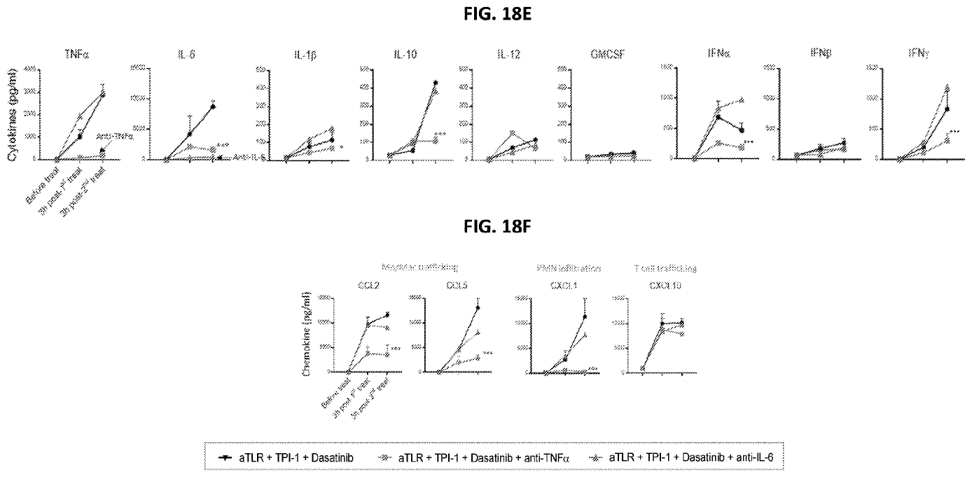

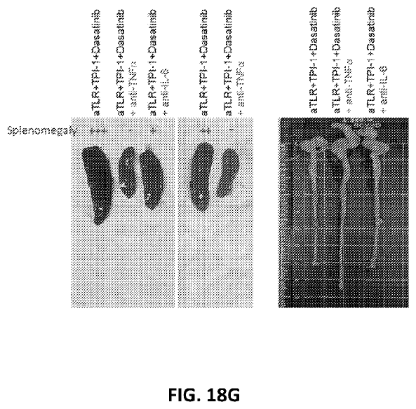

Moreover, it was found that SHP-1 inhibitor, when administered in an intermittent dosing, is able to achieve comparably remarkable anti-tumor effects as SHP-1 inhibitor in a continuous dosing, while accomplishing significantly less side effects (such as anemia, kidney damage, and liver damage). See FIGS. 11A-11D. This is highly striking in view of the severe adverse effects associated with SHP-1 inhibitor demonstrated in previous studies. Furthermore, administration of an agent that reduces systemic inflammation (e.g., an anti-TNFα mAb) further curbs down systemic inflammation and reduces adverse toxicity. See FIGS. 18A-18G.

Accordingly, this application provides novel methods that can effectively rewire tumor condition-imposed immunosuppression and license innate and adaptive immunity against cancer, thereby achieving a remarkable anti-tumor efficacy.

### I. DEFINITIONS

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “individual,” “subject,” or “patient” is used synonymously herein to describe a mammal, including humans. An individual includes, but is not limited to, human, bovine, horse, feline, canine, rodent, or primate. In some embodiments, the individual is human. In some embodiments, an individual suffers from a disease, such as cancer. In some embodiments, the individual is in need of treatment.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or individual.

As used herein, the term “intermittent” or “intermittently” in the context of dosing refers to a non-continuous dosing such as shown in FIG. 11A (lower panel), FIG. 12A, FIG. 13A, and FIG. 14A. In some cases, “intermittent” dosing refers to a dosing where a) the SHP-1 inhibitor is administered less than 12 consecutive days (e.g., less than 11, 10, 9, 8, 7, 6, 5, 4 and 3 days), AND b) the SHP-1 inhibitor is administered at least two times, and the two administrations are separated by at least one day (i.e., Day 1 and Day 3). In some embodiments, the SHP-1 inhibitor is administered daily for no more than three consecutive days, and at least twice that is separated by at least one day.

As used herein, the term “cycle” in the context of dosing refers to a time period during which there is at least one administration of a SHP-1 inhibitor. Day 1 of a cycle is defined as the day when the first administration of a SHP-1 inhibitor happens during that time period. When there are a few daily consecutive administrations of the SHP-1 inhibitor, Day 1 of the cycle is defined as the day when first administration among the few daily consecutive administrations happens. The last day of the cycle is defined as the day before the next non-consecutive administration of the SHP-1 inhibitor happens. See FIG. 12A and FIG. 14A for exemplary cycles. The cycles do not have to have the same length of time. For example, the first cycle can have five days, and the second cycle have seven days. Each cycle may have different numbers of administrations of the SHP-1 inhibitor. For example, the first cycle, which may have five days, may have one administration of the SHP-1 inhibitor, and the second cycle, which may have seven days, may have two administrations of the SHP-1 inhibitor.

As used herein the term “immunogenic” is the ability to elicit an immune response, e.g., via T-cells, B cells, or both.

As used herein, “treatment” or “treating” is an approach for obtaining beneficial or desired results including clinical results. For purposes of this invention, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

It should be noted that, as used in the specification and t e appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### II. METHODS OF TREATMENT

The present application in one aspect provides methods of treating a cancer by administering a SHP-1 inhibitor such as TPI-1 or an analog or a derivative thereof. SHP-1 inhibitor (e.g., TPI-1 or an analog or a derivative thereof) described herein comprises any agent that comprises a SHP-1 inhibitor moiety (e.g., an agent comprising TPI-1 moiety or a derivative or analog thereof moiety). In some embodiments, the SHP-1 inhibitor comprises TPI-1. In some embodiments, the individual being treated has been subject to, is being subject to, or is about to be subject to a pro-inflammatory agent such as any of those described herein. In some embodiments, the individual is under an inflammation reaction or has an ongoing infection.

In some embodiments, the method comprises administering both a SHP-1 inhibitor and a pro-inflammatory agent into the individual. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises systemically administering the SHP-1 inhibitor.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof), wherein the individual a) has been subject to, is being subject to, or is about to be subject to a pro-inflammatory agent (e.g., a TLR agonist, e.g., R848, e.g., a radiation therapy), or b) is under an inflammation reaction or has an ongoing infection, and wherein the SHP-1 inhibitor is administered systemically (e.g., intravenously or subcutaneously). In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the SHP-1 inhibitor is administered at an interval of no more than once every two days. In some embodiments, the SHP-1 inhibitor is administered no less than two times and no more than 5 times within ten consecutive days (e.g., twice in ten days, three times in ten days, four times in ten days, or five times in ten days). In some embodiments, the SHP-1 inhibitor is administered simultaneously with the pro-inflammatory agent. In some embodiments, the SHP-1 inhibitor is administered concurrently with the pro-inflammatory agent. In some embodiments, the SHP-1 inhibitor and the pro-inflammatory agent are administered sequentially and within 2 weeks (e.g., within 10 days, 7 days, 6 days, 5 days, 4 days, 3 days, 2 days, or the same day). In some embodiments, the SHP-1 inhibitor has a half-life of no more than about 10 days (e.g., no more than about 7 days, 5 days, 4 days, or 3 days). In some embodiments, the SHP-1 inhibitor is effective in inhibiting more than 50% of the SHP-1 activity for no more than about 7 days (e.g., about 5 days, 4 days, or 3 days). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the method further comprises locally (e.g., intratumorally) administering the pro-inflammatory agent into the individual. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the pro-inflammatory agent comprises a TLR agonist (e.g., R848) and a pro-inflammatory cytokine (e.g., IFN-gamma). In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a pro-inflammatory agent (e.g., a TLR agonist, e.g., R848, e.g., a radiation therapy), and wherein the method comprises intravenous or subcutaneous administration of the SHP-1 inhibitor, optionally wherein the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the SHP-1 inhibitor is administered twice (e.g., two executive days) every seven to twenty days. In some embodiments, the SHP-1 inhibitor is administered three times (e.g., three executive days) every ten to twenty days. In some embodiments, the SHP-1 inhibitor is administered at an interval of no more than once every two days. In some embodiments, the SHP-1 inhibitor is administered no less than two times and no more than 5 times within ten consecutive days (e.g., twice in ten days, three times in ten days, four times in ten days, or five times in ten days). In some embodiments, the SHP-1 inhibitor is administered simultaneously with the pro-inflammatory agent. In some embodiments, the SHP-1 inhibitor is administered concurrently with the pro-inflammatory agent. In some embodiments, the SHP-1 inhibitor and the pro-inflammatory agent are administered sequentially and within 2 weeks (e.g., within 10 days, 7 days, 6 days, 5 days, 4 days, 3 days, 2 days, or the same day). In some embodiments, the SHP-1 inhibitor has a half-life of no more than about 10 days (e.g., no more than about 7 days, 5 days, 4 days, or 3 days). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the method further comprises locally (e.g., intratumorally) administering the pro-inflammatory agent into the individual. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a pro-inflammatory agent (e.g., a TLR agonist, e.g., R848, e.g., a radiation therapy), and wherein the method comprises intravenous or subcutaneous administration of the SHP-1 inhibitor, optionally wherein the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual for at least two cycles, further optionally wherein the SHP-1 inhibitor is administered for at least once in each cycle and wherein each cycle has about three to about twenty days. In some embodiments, the SHP-1 inhibitor is administered for at least twice (e.g., at least two consecutive days) in each cycle. In some embodiments, the SHP-1 inhibitor is administered for at least three times (e.g., at least three consecutive days) in each cycle. In some embodiments, the SHP-1 inhibitor is administered simultaneously with the pro-inflammatory agent. In some embodiments, the SHP-1 inhibitor is administered concurrently with the pro-inflammatory agent. In some embodiments, the SHP-1 inhibitor and the pro-inflammatory agent are administered sequentially and within 2 weeks (e.g., within 10 days, 7 days, 6 days, 5 days, 4 days, 3 days, 2 days, or the same day). In some embodiments, the SHP-1 inhibitor has a half-life of no more than about 10 days (e.g., no more than about 7 days, 5 days, 4 days, or 3 days). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the method further comprises locally (e.g., intratumorally) administering the pro-inflammatory agent into the individual. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising intravenously, subcutaneously and/or intratumorally administering to the individual a SHP-1 inhibitor (e.g., TPI-1 or an analog or a derivative thereof) and a pro-inflammatory agent (e.g., a TLR agonist, e.g., R848, e.g., a radiation therapy), optionally wherein the SHP-1 inhibitor is effective in inhibiting more than 50% of the SHP-1 activity for no more than about 5 days, and optionally wherein the method comprises administering the SHP-1 inhibitor to the individual at an interval of no more than once every three days for at least twice (e.g., at least 3, 4, 5, or 6 times). In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the SHP-1 inhibitor is administered at an interval of no more than twice every seven to twenty days. In some embodiments, the SHP-1 inhibitor is administered at an interval of no more than three times every seven to twenty days. In some embodiments, the SHP-1 inhibitor is administered for a period of at least fourteen to twenty days at an interval of about 1-3 times every seven to twenty days. In some embodiments, the SHP-1 inhibitor is administered at least about 2, 3, 4, 5, or 6 times in a period of about fourteen to about forty days (e.g., about fourteen to about twenty days). In some embodiments, the SHP-1 inhibitor is administered simultaneously with the pro-inflammatory agent. In some embodiments, the SHP-1 inhibitor is administered concurrently with the pro-inflammatory agent. In some embodiments, the SHP-1 inhibitor and the pro-inflammatory agent are administered sequentially and within 2 weeks (e.g., within 10 days, 7 days, 6 days, 5 days, 4 days, 3 days, 2 days, or the same day). In some embodiments, the SHP-1 inhibitor has a half-life of no more than about 10 days (e.g., no more than about 7 days, 5 days, 4 days, or 3 days). In some embodiments, the SHP-1 inhibitor is effective in inhibiting more than 50% of the SHP-1 activity for no more than about 7 days (e.g., about 5 days, 4 days, or 3 days). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the method further comprises locally (e.g., intratumorally) administering the pro-inflammatory agent into the individual. In some embodiments, the SHP-1 inhibitor is administered systemically, and the pro-inflammatory agent is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising intravenously, subcutaneously and/or intratumorally administering to the individual a SHP-1 inhibitor (e.g., TPI-1 or an analog or a derivative thereof) and a pro-inflammatory agent (e.g., a TLR agonist, e.g., R848, e.g., a radiation therapy), wherein the SHP-1 inhibitor is effective in inhibiting more than 50% of the SHP-1 activity for no more than about 5 days (e.g., for no more than 5, 4, or 3 days), and wherein the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered for at least twice (e.g., at least two consecutive days) in each cycle. In some embodiments, the SHP-1 inhibitor is administered for at least three times (e.g., at least three consecutive days) in each cycle. In some embodiments, the SHP-1 inhibitor is administered simultaneously with the pro-inflammatory agent. In some embodiments, the SHP-1 inhibitor is administered concurrently with the pro-inflammatory agent. In some embodiments, the SHP-1 inhibitor and the pro-inflammatory agent are administered sequentially and within 2 weeks (e.g., within 10 days, 7 days, 6 days, 5 days, 4 days, 3 days, 2 days, or the same day). In some embodiments, the SHP-1 inhibitor has a half-life of no more than about 10 days (e.g., no more than about 7 days, 5 days, 4 days, or 3 days). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the method further comprises locally (e.g., intratumorally) administering the pro-inflammatory agent into the individual. In some embodiments, the SHP-1 inhibitor is administered systemically, and the pro-inflammatory agent is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering (e.g., intravenously, subcutaneously and/or intratumorally) to the individual a SHP-1 inhibitor (e.g., TPI-1 or an analog or a derivative thereof) and immune cells (such as any of the immune cells described herein). In some embodiments, the individual has been subject to, is being subject to, or is about to be subject to a pro-inflammatory agent (e.g., a TLR agonist, e.g., R848, e.g., a radiation therapy). In some embodiments, the individual is under an inflammation reaction or has an ongoing infection. In some embodiments, there is provided a method of treating a cancer (e.g., a solid tumor, e.g., a hematological cancer, e.g., a late stage cancer) in an individual, comprising administering (e.g., intravenously, subcutaneously and/or intratumorally) to the individual a SHP-1 inhibitor (e.g., TPI-1 or an analog or a derivative thereof), a pro-inflammatory agent (e.g., a TLR agonist, e.g., R848, e.g., a radiation therapy), and immune cells. In some embodiments, the immune cells are derived from the same individual. In some embodiments, the immune cells comprise monocytes or macrophages. In some embodiments, the immune cells comprise T cells (e.g., CAR-T cells). In some embodiments, the immune cells comprise NK cells (e.g., CAR-NK cells). In some embodiments, the immune cells comprise neutrophils (e.g., CAR-expressing neutrophils cells). In some embodiments, the immune cells comprise antigen presenting cells (APCs). In some embodiments, the immune cells are engineered to express a chimeric receptor that specifically binds to a tumor antigen. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the SHP-1 inhibitor, the immune cells, and/or the pro-inflammatory agent are administered within 7, 6, 5, 4, 3, 2 or 1 day. In some embodiments, the SHP-1 inhibitor and the immune cells are administered within 24 hours (e.g., within 12, 8, 4, 2, or 1 hour, or within 30 minutes) of each other. In some embodiments, the SHP-1 inhibitor, the immune cells, and/or the pro-inflammatory agent are administered simultaneously. In some embodiments, the SHP-1 inhibitor, the immune cells, and/or the pro-inflammatory agent are administered concurrently. In some embodiments, the SHP-1 inhibitor, the immune cells, and/or the pro-inflammatory agent are administered sequentially. In some embodiments, the SHP-1 inhibitor is administered systemically, and the pro-inflammatory agent is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the pro-inflammatory agent comprises an agent or is selected from the group consisting of R848, 3M-852A, Motolimod, Bropirimine and Vesatolimod. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a TLR agonist (e.g., R848), wherein the SHP-1 inhibitor is administered at least twice (e.g., at least 3, 4, or 5 times). In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a TLR agonist, wherein the SHP-1 inhibitor and the TLR agonist are administered within 24 hours (e.g., within 12, 8, 4, 2, or 1 hour, or within 30 minutes) of each other. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once (e.g., at least twice or three time) in each cycle and wherein each cycle has about three to about twenty days. In some embodiments, the SHP-1 inhibitor is administered systemically (e.g., intravenously or subcutaneously) and/or locally (e.g., intratumorally). In some embodiments, the SHP-1 inhibitor and the TLR agonist are administered simultaneously, concurrently or sequentially. In some embodiments, the TLR agonist activates TLR1 or TLR2, optionally wherein the TLR agonist comprises a triacylated lipoprotein, a peptidoglycan, zymosan, and/or Pam3CSK4. In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein. In some embodiments, the TLR agonist activates TLR2, optionally wherein the TLR agonist comprises Pam3Cys, SMP-105, and/or CBLB612. In some embodiments, the TLR agonist activates TLR3, optionally wherein the TLR agonist comprises dsRNA, Poly I:C, PolyICIC, Poly-IC12U, IPH302, ARNAX, and/or MPLA. In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE. In some embodiments, the TLR agonist activates TLR5, optionally wherein the TLR agonist comprises flagellin, CBLB502, and/or M-VM3. In some embodiments, the TLR agonist activates TLR6. In some embodiments, the TLR agonist activates TLR7 or TLR8, optionally wherein the TLR agonist comprises ssRNA, CpG-A, poly G10, and/or poly G3. In some embodiments, the TLR agonist activates TLR7, optionally wherein the TLR agonist comprises bistriazolyl and/or R848. In some embodiments, the TLR agonist activates TLR8, optionally wherein the TLR agonist comprises VTX1463 and/or R848. In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IM02055, SD101, and/or ODN M362. In some embodiments, the TLR agonist activates TLR10, optionally wherein the TLR agonist comprises Pam3CSK4. In some embodiments, the TLR agonist activates TLR11, optionally wherein the TLR agonist comprises Toxoplasma gondii profilin. In some embodiments, the TLR agonist activates TLR12. In some embodiments, the TLR agonist activates TLR13, optionally wherein the TLR agonist comprises VSV. In some embodiments, the TLR agonist activates TLR1, TLR2, TLR3, TLR4, TLR7, TLR8, and/or TLR9. In some embodiments, the TLR agonist activates TLR9, TLR4 and TLR7/8. In some embodiments, the TLR agonist comprises CpG, polyL:C and/or R848. In some embodiments, the pro-inflammatory agent comprises an agent or is selected from the group consisting of R848, 3M-852A, Motolimod, Bropirimine and Vesatolimod. In some embodiments, the SHP-1 inhibitor is administered systemically, and the TLR agonist is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering TPI-1 or an analog or a derivative thereof and a TLR agonist (e.g., R848), optionally wherein the TLR agonist activates one or more TLRs selected from the group consisting of TLR9, TLR4, TLR7 and TLR8. In some embodiments, the TPI-1 or an analog or a derivative thereof and the TLR agonist are administered within the same day. In some embodiments, the TPI-1 or an analog or a derivative thereof is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the TPI-1 or an analog or a derivative thereof and/or the TLR agonist are administered at least twice (e.g., at least three, four, five or six times). In some embodiments, the TPI-1 or an analog or a derivative thereof and the TLR agonist are administered at least two cycles (e.g., at least three cycles), optionally wherein the TPI-1 or an analog or a derivative thereof and TLR agonist are administered within the same day for at least two consecutive days (e.g., at least three consecutive days) in each cycle. In some embodiments, each cycle has about seven to about twenty days. In some embodiments, the TLR agonist activates a TLR on a macrophage, optionally wherein the TLR comprises TLR9. In some embodiments, the TLR agonist activates at least two TLRs (e.g., TLR4, TLR7, TLR8, or TLR9). In some embodiments, the TLR agonist activates at least three TLRs (e.g., TLR9, TLR4 and TLR7/8). In some embodiments, the TLR agonist comprises CpG, polyL:C and/or R848. In some embodiments, the pro-inflammatory agent comprises an agent or is selected from the group consisting of R848, 3M-852A, Motolimod, Bropirimine and Vesatolimod. In some embodiments, the TPI-1 or an analog or a derivative thereof is administered systemically, and the TLR agonist is administered intratumorally. In some embodiments, the TPI-1 or an analog or a derivative thereof is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a STING activator (e.g., cGAMP, e.g., MSA-2), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a STING activator (e.g., cGAMP, e.g., MSA-2), optionally wherein the SHP-1 inhibitor and the STING activator are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor and the STING activator are administered sequentially, simultaneously, or concurrently. In some embodiments, the STING activator is a cyclic-guanosine monophosphate-adenosine monophosphate (cGAMP, e.g., 3′3′ cGAMP, e.g., 2′3′ cGAMP), a bacterial vector (e.g., SYNB1891, STACT-TREX-1), a CDN compounds (e.g., ADU-S100, BI-STING, BMS-986301, GSK532, JNJ-4412, MK-1454, SB11285, 3′3′-cyclic AIMP), a non-CDN small molecule (e.g., ALG-031048, E7755, JNJ-'6196, MK-2118, MSA-1, MSA-2, SNX281, SR-717, TAK676, TTI-10001), a nanovaccine (e.g., PC7A NP, cCAMP-NP, ONM-500) or an antibody-drug conjugate (e.g., XMT-2056, CRD-5500). In some embodiments, the SHP-1 inhibitor is administered systemically, and the STING activator is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a radiation therapy, optionally wherein the method comprises administering the SHP-1 inhibitor to the individual for at least two cycles, wherein the SHP-1 inhibitor is administered for at least once in each cycle and wherein each cycle has about three to about twenty days. In some embodiments, the method comprises administering the SHP-1 inhibitor to the individual at an interval of no more than once every three days for at least twice. In some embodiments, days, the SHP-1 inhibitor is administered at least three times. In some embodiments, the SHP-1 inhibitor is administered systemically (e.g., intravenously, e.g., subcutaneously) and/or locally (e.g., intratumorally). In some embodiments, the SHP-1 inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a radiation therapy, wherein the radiation therapy comprises irradiation at a site that is different from the site of the cancer to be treated. In some embodiments, the SHP-1 inhibitor is administered at least twice (at least three, four, five, or six times). In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors. In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT). In some embodiments, the radiation therapy is brachytherapy, optionally comprising interstitial brachytherapy, intracavitary brachytherapy, intraluminal radiation therapy, and radioactively tagged molecules given intravenously. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering TPI-1 or an analog or a derivative thereof and a radiation therapy. In some embodiments, the TPI-1 or an analog or a derivative thereof is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the TPI-1 or an analog or a derivative thereof and the radiation therapy are administered within the same day. In some embodiments, the TPI-1 or an analog or a derivative thereof and/or the radiation therapy are administered at least twice (e.g., at least three, four, five or six times). In some embodiments, the TPI-1 or an analog or a derivative thereof and the radiation therapy are administered at least two cycles (e.g., at least three cycles), optionally wherein the TPI-1 or an analog or a derivative thereof and the radiation therapy are administered within the same day for at least two consecutive days (e.g., at least three consecutive days) in each cycle. In some embodiments, each cycle has about seven to about twenty days. In some embodiments, the SHP-1 inhibitor and the radiation therapy are administered within 24 hours (e.g., within 12, 8, 4, 2, or 1 hour, or within 30 minutes) of each other. In some embodiments, the radiation therapy comprises irradiation at site of the cancer to be treated. In some embodiments, the radiation therapy comprises irradiation at a site that is different from the site of the cancer to be treated. In some embodiments, the dose of the radiation therapy is insufficient to kill tumor cells. In some embodiments, the TPI-1 or an analog or a derivative thereof is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a PAMP/DAMP activator, optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a PAMP/DAMP activator, optionally wherein the SHP-1 inhibitor and the PAMP/DAMP activator are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the pro-inflammatory agent is a PAMP activator. In some embodiments, the PAMP activator is triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, or toxoplasma LPS. In some embodiments, the pro-inflammatory agent is a DAMP activator. In some embodiments, the DAMP activator is defensins, HSP60, HSP70, messenger RNA, low-molecular-weight hyaluronic acid, fibrinogen, fibronectin, fx1-defensin, heparan sulfate, HSP60, HSP70, HSP90, HMGB1, or unmethylated CpG DNA. In some embodiments, the SHP-1 inhibitor is administered systemically, and the PAMP/DAMP activator is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a checkpoint inhibitor (e.g., an anti-PD-1 agent, an anti-PD-L1 agent, or an anti-CTLA-4 agent), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a checkpoint inhibitor (e.g., an anti-PD-1 agent, an anti-PD-L1 agent, or an anti-CTLA-4 agent), wherein the SHP-1 inhibitor and the checkpoint inhibitor are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1(M-CSF)/CSF-1R, IL-1/IL-1R3 (IL-1RAP), IL-8, SEMA4D, Ang-2, CLEVER-1, Axl, or phosphatidylserine. In some embodiments, the checkpoint inhibitor comprises or is lipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), or Bavituximab. In some embodiments, the SHP-1 inhibitor is administered systemically, and the checkpoint inhibitor is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a pro-inflammatory cytokine (e.g., IL-1b, IL-18, IL-6, and/or TNFα), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a pro-inflammatory cytokine (e.g., IL-1b, IL-18, IL-6, and/or TNFα), wherein the SHP-1 inhibitor and the pro-inflammatory cytokine are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the pro-inflammatory cytokine promotes the M1 macrophages. In some embodiments, the pro-inflammatory cytokine comprises or is TNF, IFNγ, and/or GM-CSF. In some embodiments, the pro-inflammatory cytokine comprises IFNγ. In some embodiments, the pro-inflammatory cytokine comprises IL-1. In some embodiments, the pro-inflammatory cytokine comprises TNF-α. In some embodiments, the pro-inflammatory cytokine comprises IL-6. In some embodiments, the SHP-1 inhibitor is administered systemically, and the pro-inflammatory cytokine is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a chemotherapeutic agent (e.g., azathioprine), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a chemotherapeutic agent (e.g., azathioprine), wherein the SHP-1 inhibitor and the chemotherapy are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the chemotherapeutic agent is an alkylating agent. In some embodiments, the alkylating agent is selected from the group consisting of nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa). In some embodiments, the chemotherapeutic agent is an antimetabolite. In some embodiments, the antimetabolite is selected from the group consisting of icytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), capecitabine (prodrug of 5-FU)). In some embodiments, the chemotherapeutic agent is an antimicrotubular agent. In some embodiments, the antimmicrotubular agent is selected from the group consisting of topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), antibiotics (e.g., actinomycin D, bleomycin, daunomycin). In some embodiments, the chemotherapeutic agent is hydroxyurea, tretinoin, arsenic trioxide, or a proteasome inhibitor (e.g., bortezomib). In some embodiments, the SHP-1 inhibitor is administered systemically, and the chemotherapeutic agent is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a cancer vaccine, optionally wherein the SHP-1 inhibitor is administered at least twice. In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a cancer vaccine, wherein the SHP-1 inhibitor and the cancer vaccine are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the cancer vaccine comprises a cell-based vaccine, a peptide-based vaccine, a viral-based vaccine, and/or a nucleic acid-based vaccine. In some embodiments, the SHP-1 inhibitor is administered systemically, and the cancer vaccine is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and an oncolytic virus, optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a oncolytic virus, wherein the SHP-1 inhibitor and the oncolytic virus are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, L1BR1, HF10, T-VEC, Orien X010). In some embodiments, the oncolytic viruses comprises JX-593, Coxsackievirus A21 (CVA21), marabi virus or its MG1 variant, DNX2440 adenovirus, fowl pox virus, or Sendai virus. In some embodiments, the SHP-1 inhibitor is administered systemically, and the oncolytic virus is administered intratumorally. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a sound treatment (e.g., high intensity focused ultrasound (HIFU), e.g., low intensity focused ultrasound (LIPUS)), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a sound treatment (e.g., high intensity focused ultrasound (HIFU), e.g., low intensity focused ultrasound (LIPUS)), wherein the SHP-1 inhibitor and the sound treatment are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor is administered systemically, and the method comprises administering the sound treatment at the site of the cancer to be treated. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a magnetic therapy (e.g., pulsed magnetic field, e.g., static magnetic field), optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a magnetic therapy (e.g., pulsed magnetic field, e.g., static magnetic field), wherein the SHP-1 inhibitor and the magnetic therapy are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor is administered systemically, and the method comprises administering the magnetic treatment at the site of the cancer to be treated. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and an electrical treatment or electrochemical treatment, optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a electrical or electrochemical treatment, wherein the SHP-1 inhibitor and the electrical treatment or electrochemical treatment are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor is administered systemically, and the method comprises administering the electrical treatment or electrochemical treatment at the site of the cancer to be treated. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and an electrostatic treatment, optionally wherein the SHP-1 inhibitor is administered at least twice (at least three, four, five, or six times). In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof) and a an electrostatic treatment, wherein the SHP-1 inhibitor and the an electrostatic treatment are administered within 24 hours (e.g., within 12, 8, 4, 2, or 1 hour, or within 30 minutes) of each other. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the SHP-1 inhibitor is administered systemically, and the method comprises administering the electrostatic treatment at the site of the cancer to be treated. In some embodiments, the SHP-1 inhibitor is administered systemically and intratumorally. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof), wherein the individual is selected for treatment based upon the individual having an ongoing inflammation reaction. In some embodiments, the individual has an acute inflammation reaction. In some embodiments, the inflammation reaction is in the tumor. In some embodiments, the inflammation reaction is at a site distinct from the tumor. In some embodiments, the individual has an inflammation reaction when an inflammation reaction where there are at least two (e.g., two, three, four or five) events selected from the group consisting of a) an increase in one or more (e.g., at least one, two, three, four, five) inflammatory cytokines (such as IFNγ, IL-12b, TNFα, IL-6, IL-1b, IFN-a1, IFN-a2, IFN-b1), b) a decrease in one or more (e.g., at least one, two or three) anti-inflammatory cytokine (such as TGFb1, TGFb2, TGFb3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages). In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the SHP-1 inhibitor is administered at least twice (e.g., at least three, four, five or six times). In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, there is provided a method of treating a cancer (e.g., a solid tumor, e.g., a hematological cancer, e.g., a late stage cancer) in an individual, comprising administering to the individual a SHP-1 inhibitor (e.g., TPI-1 or an analog or a derivative thereof), wherein the individual is selected for treatment based upon the individual having an ongoing immunogenic cell death (ICD). In some embodiments, the individual has ICD when a sample from the cancer has a higher level of one or more (e.g., at least 10%, 20%, 30%, 40%, 50%, 60%, 70%, 80%, 90% more) DAMPs than a reference sample (e.g., a corresponding sample in a healthy control, e.g., a sample from the cancer prior to the administration of a therapy that induces ICD. In some embodiments, the SHP-1 inhibitor is administered intermittently. In some embodiments, the SHP-1 inhibitor is administered daily for no more than three or two consecutive days, and optionally at least twice which are separated by at least one day. In some embodiments, the SHP-1 inhibitor is administered at least three, four, or five times. In some embodiments, at least two SHP-1 inhibitor administration is separated by two, three, four, five, six, seven, eight, nine, or two days. In some embodiments, each of the SHP-1 inhibitor administration is separated by at least one day from the proceeding or following SHP-1 inhibitor administration. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs). In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1). In some embodiments, the SHP-1 inhibitor is selected from the group consisting of TPI-1 or an analog or a derivative thereof, vitamin E derivative, phomoxanthone A (PXA), and a PKCθ activator. In some embodiments, the SHP-1 inhibitor is administered at least twice (e.g., at least three, four, five or six times). In some embodiments, the method comprises administering the SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the SHP-1 inhibitor is administered systemically (e.g., intravenously, e.g., subcutaneously) and/or locally (e.g., intratumorally). In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

In some embodiments, the present application provides a method of treating a cancer (e.g., a solid tumor, e.g., a hematological cancer, e.g., a late stage cancer) in an individual, comprising administering to the individual a) monocytes or macrophages deficient in SHP-1 expression or activation and b) a pro-inflammatory agent (e.g., a TLR agonist, e.g., R848, e.g., a radiation therapy). In some embodiments, the monocytes or macrophages are derived from the same individual. In some embodiments, the monocytes or macrophages are engineered to express a chimeric receptor targeting a tumor antigen. In some embodiments, the monocytes or macrophages and the pro-inflammatory agent are administered within 24 hours (e.g., within 12, 8, 4, 2, or 1 hour, or within 30 minutes) of each other. In some embodiments, the monocytes or macrophages and the pro-inflammatory agent are administered simultaneously, concurrently, or sequentially. In some embodiments, the monocytes or macrophages are administered prior to the pro-inflammatory agent. In some embodiments, the monocytes or macrophages are administered following the pro-inflammatory agent. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

The present application also provides a method of modulating monocytes or macrophages derived from an individual having a cancer, comprising contacting the monocytes or macrophages with a SHP-1 inhibitor as described above, and a pro-inflammatory agent as described above. In some embodiments, the monocytes or macrophages are derived from the same individual. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

The present application also provides methods of activating phagocytosis against tumor cells in an individual having a tumor, comprising administering to the individual a SHP-1 inhibitor, wherein the individual a) has been subject to, is being subject to, or is about to be subject to a pro-inflammatory agent, or b) is under an inflammation reaction or has an ongoing infection. In some embodiments, the SHP-1 inhibitor is administered systemically (e.g., intravenously or subcutaneously). The present application also provides a method of activating tumor infiltrating T cells in an individual having a tumor comprising administering to the individual a SHP-1 inhibitor, wherein the individual a) has been subject to, is being subject to, or is about to be subject to a pro-inflammatory agent, or b) is under an inflammation reaction or has an ongoing infection. In some embodiments, the method comprises administering SHP-1 inhibitor to the individual at an interval of no more than once every three days for at least twice. In some embodiments, the method comprises administering the SHP-1 inhibitor to the individual for at least two cycles, wherein the SHP-1 inhibitor is administered for at least once in each cycle and wherein each cycle has about three to about twenty days. In some embodiments, the pro-inflammatory agent and the SHP-1 inhibitor are administered within 24 hours of each other. In some embodiments, the pro-inflammatory agent comprises an agent selected from the group consisting of a TLR agonist, a STING activator, a radiation therapy, a PAMP/DAMP activator, a checkpoint inhibitor, a pro-inflammatory cytokine, a chemotherapeutic agent, a bacteria component, a cancer vaccine, and an oncolytic virus. In some embodiments, the method further comprises administering to the individual an agent that reduces systemic inflammation and/or reduces inflammatory cytokine cascade or cytokine storm (e.g., an anti-TNFα antibody or an anti-IL6 antibody). In some embodiments, the method further comprises administering to the individual an anti-TNFα antibody, optionally wherein the anti-TNFα antibody is administered prior to (e.g., within two weeks, ten days, a week, 48 hours, or 24 hours), concurrently with or simultaneously with, or immediately after (within 3, 2, 1, or 0.5 hour) the administration of the SHP-1 inhibitor (e.g., TPI-1 or an analog or derivative thereof) and/or the pro-inflammatory agent. In some embodiments, the SHP-1 inhibitor comprises TPI-1.

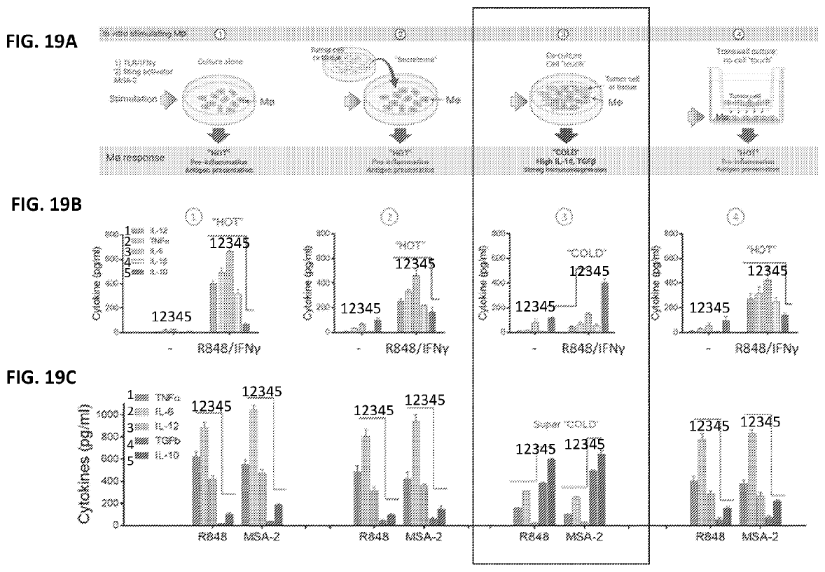

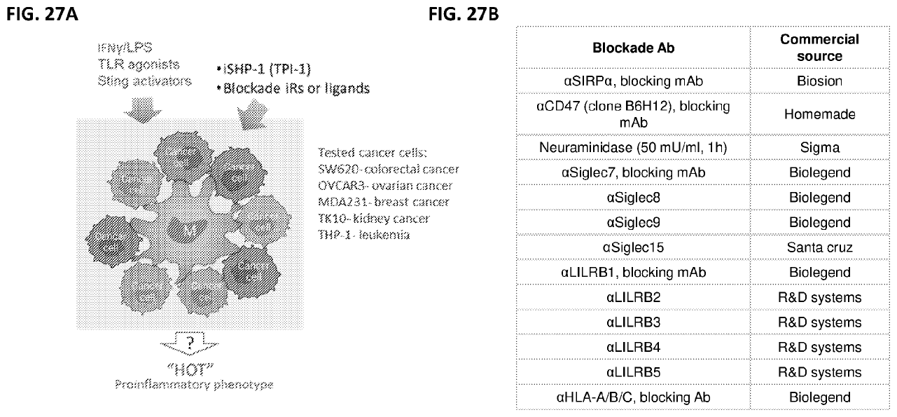

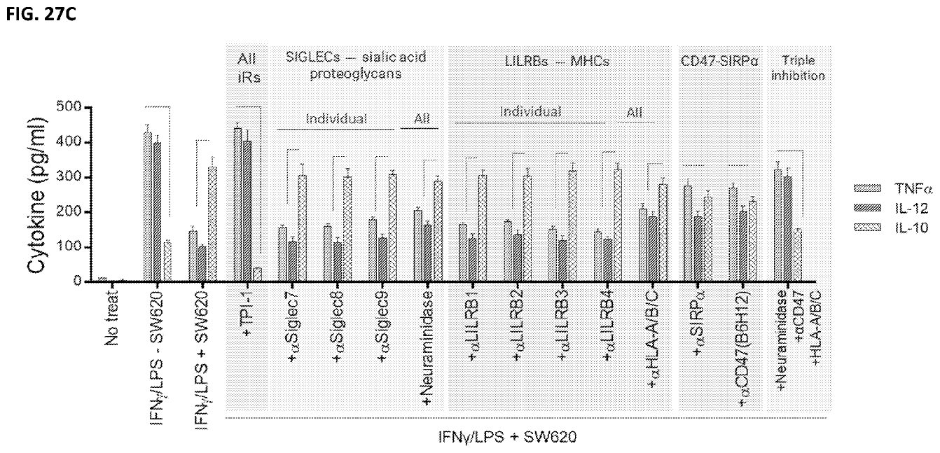

It was also found that the strong inhibitory regulation via the intratumoral iRs-SHP-1 was largely dependent upon physical contact between cancer cells and macrophages. Separating cancer cells from macrophages using a transwell that allows soluble factor transmitting but prevents cancer cell “touching” macrophages failed to assert strong inhibition on macrophage proinflammatory response. See e.g., FIG. 19. Combining blockers of iRs (such as antibodies, fusion proteins, or other agents that blockade a) SIGLECs from interacting sialic acid proteoglycans, b) LILRBs or MHCs for interactions, c) CD47 or SIRPα, d) lectins receptors, or e) signaling lymphocytic activation molecule family (SLAMF) receptors or their ligands) also achieve the effect of eliminating iRs-mediated inhibition, allowing macrophage activation towards proinflammatory direction as evidenced by elevated cytokines shown in FIG. 27C.

Therefore, it is also contemplated that iRs blockers (such as antibodies, fusion proteins, or other agents that blockade SIGLECs for interacting with sialic acid proteoglycan ligands, blockade LILRBs interactions with MHCs, blockade the interaction between CD47 and SIRPα, blockade interactions between lectins and lectin receptors, blockade interactions between signaling lymphocytic activation molecule family (SLAMF) receptors and their ligands, etc.), especially a combination of these blockers can be used in replacement of SHP-1 inhibitor in the methods described herein. See e.g., FIG. 27C. In some embodiments, there is provided a method of treating a cancer comprising administering at least two or three blockers that blockade different interactions selected from the group consisting of SIGLECs-sialic acid proteoglycans, LILRBs-MHCs, CD47-SIRPα, lectins-lectin receptors, signaling lymphocytic activation molecule family (SLAMF) receptors-their ligands and optionally a pro-inflammatory agent.

Blockers described herein include any agent that is a) capable of reducing the binding of the inhibitory receptor and its ligand measured by e.g., spectroscopic assays, isothermal titration calorimetry (ITC), optical biosensors such as surface plasmon resonance (SPR), biolayer interferometry (BLI), or grating-coupled interferometry (GCI), and/or b) the activation of the inhibitor receptor measured by e.g., western blot of the activated downstream signaling by at least 30%, 40%, 50%, 60%, 70%, 80%, or 90%. Exemplary blockers include e.g., blocking antibodies that bind to the iRs or their ligands.

In some embodiments, the method comprises administering into an individual in need thereof a) a blocker of CD47-SIRPα (e.g., an anti-CD47 antibody (e.g., B6H12) or an anti-SIRPα antibody), b) a blocker of LILRBs-MHCs (e.g., an antibody against LILRB1, LILRB2 and/or LILRB3, e.g., an antibody against HLA-A, HLA-B, and/or HLA-C), c) a blocker of SIGLECs-sialic acid proteoglycans (e.g., an anti-siglec9, an anti-siglec7, an anti-siglec 8, e.g., neurominidase) and optionally d) a pro-inflammatory agent (e.g., a TLR agonist, a STING activator). In some embodiments, the method comprises administering into an individual in need thereof a) neuraminidase, b) an anti-CD47 antibody, c) an anti-HLA-A/B/C, and optionally d) a pro-inflammatory agent (e.g., a TLR agonist, a STING activator). Exemplary antibodies against the iRs or their ligands can be found in FIG. 27B. In some embodiments, the individual has an ongoing infection and does not need to be treated with a pro-inflammatory agent.

### Tumor Microenvironment (TME) Immunosuppression and SHP-1 Signaling

Src homology region 2 (SH-2) domain-containing phosphatase 1 (SHP-1) is a non-receptor tyrosine phosphatase encoded by the PTPN6 gene that is located on human chromosome 12p13 and contains two promoter regions (within exon 1 and 2), giving rise to two forms of SHP-1 which differ in their N-terminal amino acid sequences but have a similar phosphatase activity. Promoter I is active in non-hematopoietic cells, while promoter II in hematopoietic-derived cells; in some epithelial cancer cells both promoters may function and generate various SHP-1-alternative transcripts. The two SHP-1 isoforms show different subcellular localizations: form I is mainly located in the nucleus, while form II is in the cytoplasm, suggesting that they have different targets.

SHP-1 is a 595 amino acid protein composed of two tandem N-terminal SH2 domains (N-SH2 and C-SH2), a classic catalytic protein tyrosine phosphatase (PTP) domain, and a C-terminal tail containing several phosphorylation sites. Its crystal revealed a structure in which the N-SH2 is bound to the catalytic site of the protein through charge-charge interaction. In this auto-inhibited inactive state the access of substrates to the active site is prevented, but binding of phosphotyrosine residues to the SH2 domains causes a conformational change that impairs the interaction between the N-SH2 and the catalytic domains. This opens the conformation to allow the access of substrate and is further stabilized by new interactions between SH2 domains and the catalytic domain. These molecular rearrangements determine a sophisticated regulatory mechanism controlled by substrate recruitment.

An additional mechanism of activation is mediated by the phosphorylation of amino acids within the C-terminal tail. So far, three phosphorylation sites have been found, two tyrosine (Tyr536 and Tyr564) and a serine (Ser591) residues. Tyr536 and Tyr564 become phosphorylated upon various stimuli (i.e., insulin stimulation or apoptosis inducers), giving rise to an increased SHP-1 activity. The molecular mechanism is not clear, although it has been proposed that Tyr phosphorylations could lead to interaction with the N-SH2 domain, releasing the inhibitory effect of this domain on the PTPase activity. SHP-1 activity can also be negatively regulated by protein kinase C (PKC) or mitogen-activated protein kinases (MAPKs) through phosphorylation at Ser591, whose mechanism of inhibition has not been well-characterized.

Protein-tyrosine phosphorylation is a reversible post-translational modification, tightly regulated by both kinases and phosphatases. Any deviation in the phosphorylation/dephosphorylation balance can promote the intracellular accumulation of tyrosine-phosphorylated proteins, which cause an altered regulation of cellular processes including cell growth, migration, invasion, differentiation, survival, and cellular trafficking. In this scenario, SHP-1 acts as a classical tumor suppressor, mainly involved in the homeostatic maintenance of potentially all these processes. SHP-1 function is indeed altered in both solid and hematological human cancers through somatic mutations or epigenetic mechanisms. Besides its well-documented role in the regulation of hematopoietic cell biology, SHP-1 has now been correlated to a number of signal transduction pathways relevant to cancer pathogenesis and progression.

However, inhibition of SHP-1 risks severe adverse effects. Studies of SHP-1 genetically deficient mice, the motheaten mice (me/me or mev/mev), reveal critical immunological abnormalities and hyperactivation of immune cells associated with the global loss of the SHP-1 (6, 7). The motheaten mice usually succumb to life-threatening autoimmune inflammatory conditions at the early age. Even partial depletion of SHP-1 in WT mice after they grew to adults led to features of inflammatory disease, causing extensive lung inflammation and splenomegaly. Like a double-edged sword, inhibition of SHP-1, despite the potential of empowering anti-cancer immunity, inevitably endangers hosts for heightened inflammatory response, cytokine storm and autoimmunity.

Inhibitors targeting the SHP-1 phosphatase activity have been under development for some times, and some have now entered preclinical studies, including NSC-87877, sodium stibogluconate (SSG), tyrosine phosphatase inhibitor 1 (TPI-1 or an analog or a derivative thereof), and suramine; however, only a few of them have been shown to be active in experimental tumor models. SSG has been through Phase I trials for both malignant melanoma (NCT00498979) and advanced malignancies (NCT00629200); the drug was administrated in combination with interferons followed or not by chemotherapy treatment. Unfortunately, no effect was seen against tumor development, with the most common toxic side-effects being thrombocytopenia, elevated serum lipase, fatigue, fever, chills, anemia, hypokalemia, pancreatitis, and skin rash (observed in up to 68% of patients). At present, no SHP-1 inhibitor is under Phase II trial.

An Agent that Reduces Systemic Inflammation

In some cases, individuals develop systemic inflammation, i.e., cytokine release syndrome (CRS) after receiving (e.g.) immunotherapeutic treatment, however the inflammatory disorder is not fully understood. CRS can be induced by direct target cell lysis and the consecutive release of cytokines like TNFα or IFNγ, or by activation of T cells due to therapeutic stimuli that is followed by subsequent cytokine release. These cytokines trigger a chain reaction due to the activation of innate immune cells like macrophages and endothelial cells, which then induces further cytokine release. In particular, IL6, IL10, and IFNγ are most commonly found to be elevated in patients with CRS.

The methods described herein can further comprises administration of an agent that reduces systemic inflammation (including, for example, an agent that reduces inflammatory cytokine cascade or cytokine storm), in order to curb down systemic inflammation and reduce adverse toxicity. The agents that reduce systemic inflammation include, but are not limited to, inhibitors of TNFα, IL6, IL10, and IFNγ. In some embodiments, the agent that reduces systemic inflammation is administered simultaneously with the SHP-1 inhibitor. In some embodiments, the agent that reduces systemic inflammation is administered sequentially (e.g., prior to or after) with the SHP-1 inhibitor. In some embodiments, the administration of the agent that reduces systemic inflammation follows the same dosing schedule as the SHP-1 inhibitor. In some embodiments, the agent that reduces systemic inflammation is administered at a sub-therapeutic dose, namely, at a dose that is lower than an effective amount for treating a disease when administered alone. In some embodiments, the administration of the agent that reduces systemic inflammation allows more frequent administration of the SHP-1 inhibitor and/or the proinflammatory agent (e.g., daily, once every two days, once every three days, etc.).

The agent can include any anti-inflammatory agent known in the art, including inhibitors of or antagonists to pro-inflammatory agents. For example, the agent can be an inhibitor or antagonist, including but not limited to, a small molecule inhibitor, a neutralizing antibody, a receptor blockade antibody, a soluble receptor, a targeting short interfering RNA (siRNA), a chemical inhibitor of mRNA stability, derivatives thereof, and any combination thereof, including combinations of agents targeting one or more molecules (e.g., targeting via the inhibition of TNFα alone, IL6 alone, TNFα and IL6 in combination).

### Anti-TNFα Antagonist

TNFα, a major proinflammatory cytokine, is secreted by activated macrophages, monocytes and lymphocytes. Inventors surprisingly found that the administration of an anti-TNFα antibody to an individual who has been administered with a SHP-1 inhibitor and a proinflammatory agent alleviates toxicity caused by systemic inflammation without compromising the efficacy of the therapeutic agents.

The methods of the present application therefore in some embodiments comprises administration of TNFα inhibitor, e.g., an anti-TNFα antagonist (e.g., in the context where the proinflammatory agent is not TNFα). In some embodiments, the TNFα inhibitor is selected from the group consisting of a small molecule inhibitor, a neutralizing antibody, a TNFα receptor blockade antibody, a soluble TNFα receptor, a TNFα-targeting short interfering RNA (siRNA), a chemical inhibitor of TNFα mRNA stability, an inhibitor of TNFα converting enzyme (TACE), and derivatives thereof. In some embodiments, the TNFα inhibitor is an anti-TNFα neutralizing antibody. In some embodiments, the TNFα inhibitor is an anti-TNFα receptor blockade antibody. In some embodiments, the anti-TNFα antibody is a monoclonal antibody. In some embodiments, anti-TNFα antibody is a chimeric, humanized, and/or fully human antibody.

Suitable antibodies for use in the methods provided herein include, but are not limited to, Remicade® (Infliximab (Centocor)), and those antibodies described, for example, in U.S. Pat. Nos. 6,835,823; 6,790,444; 6,284,471; 6,277,969; 5,919,452; 5,698,195; 5,656,272; and 5,223,395 and in EP Patent No. 0610201, the contents of each of which are hereby incorporated by reference in their entirety, or antibodies that bind to the same epitope as Remicade®. Others suitable anti-TNFα antibodies for use in the methods provided herein are, by way of non-limiting example, Humira (Adalimumab (Abbott Laboratories, Esai)) as described in U.S. Pat. Nos. 6,090,382; 6,258,562; or 6,509,015 and related patents and applications, the contents of which are hereby incorporated by reference in their entirety; Simponi™ (Golimimab, CNTO 148 (Centocor)) as described in PCT Publication No. WO 02/12502 and related patents and applications, the contents of which are hereby incorporated by reference in their entirety; ART621 (Arana Therapeutics), SSS 07 (Epitopmics and 3SBio) or antibodies that bind to the same epitope as Humira, Simponi, ART621, or SSS 07.

In some embodiments, the TNFα inhibitor, e.g., anti-TNFα antagonist, is a fusion protein. Suitable fusion proteins for use in the methods provided herein include, but are not limited to, Enbrel (Etanercept (Amgen)) and other fusion proteins or fragments thereof described in U.S. Pat. No. 5,712,155, PCT Publication No. WO 91/03553, and related patents and applications, the contents of which are hereby incorporated by reference in their entirety.

In some embodiments, the TNFα inhibitor, e.g., anti-TNFα antagonist, is a modified antibody antagonist or a non-antibody-based antagonist. Such antagonists include advanced antibody therapeutics, such as antibody fragments including, but not limited to, Cimzia™ (Certolizumab pegol, CDP870 (Enzon)), bispecific antibodies, Nanobodies® such as ABX 0402 (Ablynx), immunotoxins, and radiolabeled therapeutics; peptide therapeutics; gene therapies, particularly intrabodies; oligonucleotide therapeutics such as aptamer therapeutics, antisense therapeutics, interfering RNA therapeutics; and small molecules such as LMP-420 (LeukoMed) as described in EP Patent No. 0767793, and related patents and applications, the contents of which are hereby incorporated by reference in their entirety.

In some embodiments, the TNFα inhibitor (e.g., an anti-TNFα antibody) is administered within two weeks, 10 days, or one week prior to the administration of the SHP-1 inhibitor and/or proinflammatory agent described herein. Exemplary TNFα inhibitors such an anti-TNFα antibody is usually stable for at least one or two weeks. In some embodiments, the TNFα inhibitor (e.g., an anti-TNFα antibody) is administered concurrently or simultaneously with the SHP-1 inhibitor and/or proinflammatory agent. In some embodiments, the TNFα inhibitor (e.g., an anti-TNFα antibody) is administered immediately after (e.g., within 1 hour or 30 minutes) the administration of the SHP-1 inhibitor and/or proinflammatory agent.

In some embodiments, the TNFα inhibitor is administered systemically. In some embodiments, the TNFα inhibitor is administered at least once a week, once every five days, once every three days, or daily. In some embodiments, the TNFα inhibitor is administered intermittently. In some embodiments, the TNFα inhibitor is administered to the individual for at least two cycles, wherein each cycle has about three to about seven days. In some embodiments, the individual does not develop cytokine release syndrome or pro-inflammatory organ damage. In some embodiments, administration of the TNFα inhibitor does not compromise or weakly compromises tumor clearance.

### Anti-IL6 Antagonist

An “anti-IL6 antagonist” or “IL6 inhibitor” refers to an agent that inhibits or blocks IL6 biological activity via binding to IL6 or IL6 receptor. In some embodiments, the anti-IL6 antagonist is an antibody. In one embodiment, the anti-IL6 antagonist is an antibody that binds IL6 receptor. Antibodies that bind IL-6 receptor include tocilizumab (including intravenous, i.v., and subcutaneous, s.c., formulations thereof) (Chugai, Roche, Genentech), satralizumab (Chugai, Roche, Genentech), sarilumab (Sanofi, Regeneron), NI-1201 (Novimmune and Tiziana), and vobarilizumab (Ablynx). In one embodiment, the anti-IL6 antagonist is a monoclonal antibody that binds IL6. Antibodies that bind IL-6 include sirukumab (Centecor, Janssen), olokizumab (UCB), clazakizumab (BMS and Alder), siltuximab (Janssen), and EBI-031 (Eleven Biotherapeutics and Roche). In one embodiment, the IL6 antagonist is olamkicept.

In some embodiments, the IL6 inhibitor is administered systemically. In some embodiments, the IL6 inhibitor is administered at least once a week, once every five days, once every three days, or daily. In some embodiments, the IL6 inhibitor is administered intermittently. In some embodiments, the IL6 inhibitor is administered to the individual for at least two cycles, wherein each cycle has about three to about seven days.

### SHP-1 Inhibitors

The SHP-1 inhibitors referred herein is an agent of any kind or sort that inhibits the expression or activation of SHP-1. In some embodiments, the SHP-1 inhibitor directly targets SHP-1. In some embodiments, the SHP-1 inhibitor targets a molecule involved in SHP-1 signaling pathway in macrophages that is distinct from SHP-1.

In some embodiments, the SHP-1 inhibitor is capable of inhibiting at least about 20% (e.g., at least 20%, 30%, 40%, or 50%) of the SHP-1 activity. In some embodiments, the SHP-1 inhibitor is capable of inhibiting at least about 20% (e.g., at least 20%, 30%, 40%, or 50%) of the SHP-1 expression.

In some embodiments, the SHP-1 inhibitor is selected from the group consisting of a small molecule, a nucleic acid (e.g., a siRNA, a shRNA, an antisense RNA, a microRNA), a nucleic acid editing system (e.g., a CRISPR system), a protein agent (e.g., an antibody agent that targets SHP-1 or activated SHP-1, e.g., a dominant negative SHP-1 or a constitutively active SHP-1 mutant), a protein agent that contains a SH2 domain (by competing for binding to ITIM motif so to inhibit SHP-1 activation), and a tyrosine kinase inhibitor that inhibit ITIM phosphorylation.

In some embodiments, the SHP-1 inhibitor does not significantly inhibit SHP-2 (e.g., does not inhibit the SHP-2 activity for more than 50%, 40%, 30%, or 20%).

In some embodiments, the SHP-1 inhibitor also inhibits SHP-2.

In some embodiments, the SHP-1 inhibitor has a half-life of no more than about 10, 9, 8, or 7 days (e.g., a half-life of no more than about 7, 6, 5, 4, 3, 2 or 1 day).

In some embodiments, the SHP-1 inhibitor is effective in inhibiting more than 50% of the SHP-1 activity for no more than about 10, 9, 8, 7, 6, or 5 days. In some embodiments, the SHP-1 inhibitor is effective in inhibiting more than 50% of the SHP-1 activity for no more than 4, 3, 2 or 1 day.

In some embodiments, the SHP-1 inhibitor is a covalent inhibitor. In some embodiments, the SHP-1 inhibitor is a noncovalent inhibitor.

In some embodiments, the SHP-1 inhibitor is a competitive inhibitor. In some embodiments, the SHP-1 inhibitor is Phomoxanthone A (PXA) or Phomoxanthone B (PXB). See e.g., Yang et al., ACS Omega. 2020 Sep. 29; 5(40):25927-25935

In some embodiments, the SHP-1 inhibitor targets the catalytic site. In some embodiments, the SHP-1 inhibitor binds to the catalytic site (e.g., covalently or competitively binds to the catalytic site). Exemplary catalytic site inhibitors include TPI-1 or TPI analogs such as those shown in Kundu et al. (e.g., TPI-1a1-10). See J Immunol. 2010 Jun. 1; 184(11): 6529-6536. Methods for screening and identifying SHP-1 inhibitors (e.g., SHP-1 inhibitors targeting the catalytic site) are known in the field. For example, recombinant protein of SHP-1 catalytic domain can be used to screen and identify SHP-1 inhibitors that target the catalytic site. SHP-1 inhibition activities can be evaluated with various methods such as rapid SHP-1 PTP assay. See “materials and methods” in Kundu et al.

In some embodiments, the SHP-1 inhibitor targets the allosteric or regulatory site. See e.g., Wang et al. J Cell Biochem. 2011 August; 112(8): 2062-2071 for the structure of SHP-1.

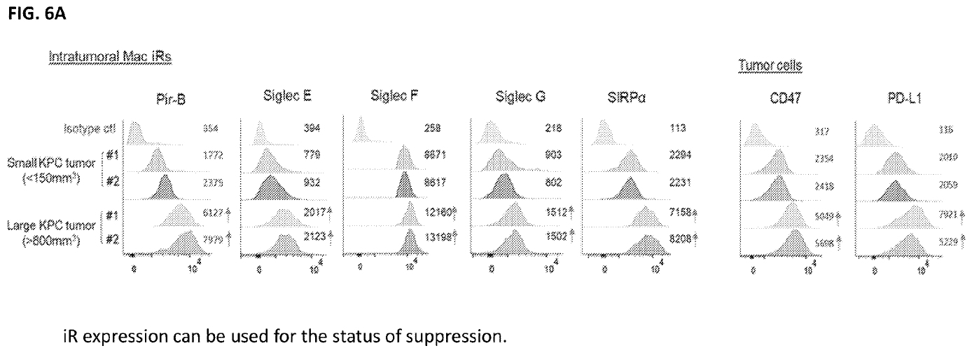

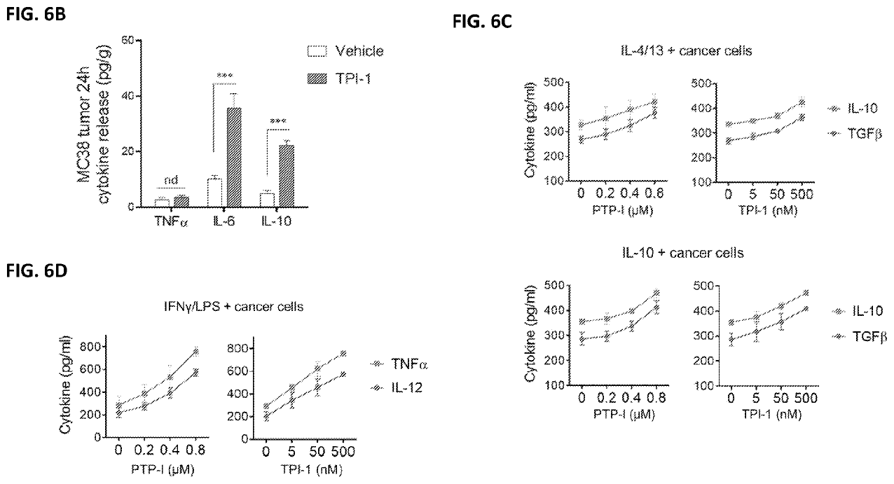

In some embodiments, the SHP-1 inhibitor is TPI-1, a derivative thereof or an analog thereof. Exemplary analogs include those disclosed in Kundu et al. (J Immunol. 2010 Jun. 1; 184(11): 6529-6536.) See, e.g., FIG. 6 of Kundu et al.

In some embodiments, the SHP-1 inhibitor comprises TPI-1.

In some embodiments, the SHP-1 inhibitor is PTP-I.

In some embodiments, the SHP-1 inhibitor is vitamin E. In some embodiments, the SHP-1 inhibitor is tocofersolan (TPGS). In some embodiments, the SHP-1 inhibitor is α-tocopherol acetate (αTA). In some embodiments, the SHP-1 inhibitor is α-tocopheryl succinate (αTOS).

In some embodiments, the SHP-1 inhibitor is phomoxanthone A (PXA).

In some embodiments, the SHP-1 inhibitor is PKCθ activator (such as PMA).

In some embodiments, the SHP-1 inhibitor is a siRNA or a shRNA that inhibits or knocks down the amount of endogenous SHP-1 protein. See e.g., WO2009/023333.

In some embodiments, the SHP-1 inhibitor is a dominant negative SHP-1 or a constitutively active SHP-1 mutant. See e.g., WO2009/023333.

In some embodiments, the SHP-1 inhibitor is a nucleic acid editing system (such as a CRISPR system). In some embodiments, the CRISPR components are introduced into the cell (e.g., the monocytes and the macrophages) but no DNA encoding a guide RNA or Cas9 are incorporated into the cell's genome. Under this approach, the CRISPR system only cleave the cell's genomic DNA for a limited period of time. See e.g., Fister et al., Front Plant Sci. 2018 Mar. 2; 9:268.

In some embodiments, the SHP-1 inhibitor is a chemical inducer of dimerization. See e.g., Buck et. al., ACS Omega. 2022 Apr. 11; 7(16):14180-14188.

In some embodiments, the SHP-1 inhibitor (e.g., TPI-1 or an analog or a derivative thereof) is administered at least two times (such as at least 3, 4, 5, or 6 times).

In some embodiments, the method comprises administering the SHP-1 inhibitor (e.g., TPI-1 or an analog or a derivative thereof) at an interval of no more than once every two days for at least twice (such as at least three times, four times, five times, or six times).

In some embodiments, the method comprises administering the SHP-1 inhibitor (e.g., TPI-1 or an analog or a derivative thereof) at an interval of no more than once every three days for at least twice (such as at least three times, four times, five times, or six times).

In some embodiments, the method comprises administering the SHP-1 inhibitor (e.g., TPI-1 or an analog or a derivative thereof) for at least two cycles. In some embodiments, SHP-1 inhibitor (e.g., TPI-1 or an analog or a derivative thereof) is administered for at least once (e.g., for twice, three times, four times) in each cycle. In some embodiments, each cycle has about three to about 50 days (e.g., about 3-40 days, about 3-30 days, about 3-20 days, about 3-15 days, about 3-10 days, or about 2-10 days).

In some embodiments, the SHP-1 inhibitor is administered systemically (e.g., orally, intravenously, subcutaneously, intraperitoneally). In some embodiments, the SHP-1 inhibitor is administered locally (e.g., intratumorally). In some embodiments, the SHP-1 inhibitor is administered both systemically and locally (e.g., intratumorally).

In some embodiments, the SHP-1 inhibitor is complexed with a delivery vehicle before being administered into the individual. In some embodiments, the delivery vehicle promotes the delivery into the tumor.

In some embodiments, the SHP-1 inhibitor modulates a monocyte or macrophage (e.g., a monocyte or macrophage derived from the individual to be treated) in vitro.

In some embodiments, the SHP-1 inhibitor and the pro-inflammatory agent below are administered within 24 hours (e.g., within 12, 8, 4, 2, or 1 hour, or within 30 minutes) of each other. In some embodiments, the SHP-1 inhibitor and the pro-inflammatory agent are administered simultaneously, concurrently, or sequentially. In some embodiments, the SHP-1 inhibitor is administered prior to the pro-inflammatory agent. In some embodiments, the SHP-1 inhibitor is administered following the pro-inflammatory agent.

### Pro-Inflammatory Agents

Infection and tissue injury are the two classic instigators of inflammation. See e.g., Medzhitov, Nature. 2008 Jul. 24; 454(7203):428-35. Pro-inflammatory agents described herein include at least two overlapping categories: 1) an agent or therapy of any kind or sort that can promote an inflammation (e.g., by promoting one or more pro-inflammatory cytokines or chemokines, inhibiting one or more anti-inflammatory cytokines or chemokines, recruiting macrophages, NK cells, neutrophils, effector T cells, or B cells to the tissue or activating any of these cells, or suppressing regulatory/suppressive immune cells such as regulatory T cells or MDSC), and 2) an agent or therapy that can cause damage of cancer cells (e.g., necrosis of cancer cells).

In some embodiments, the pro-inflammatory agent triggers a pro-inflammatory signal on macrophages. See e.g., FIG. 5A. In some embodiments, the pro-inflammatory agent activates a TLR, a TNFR, or ITAM-R. See Lionel et al., Eur J Immunol. 2011 September; 41(9): 2477-2481. The pro-inflammatory can activate a pro-inflammatory signal on macrophages via a direct manner or indirect manner. For example, a TLR agonist, which directly activates TLR on macrophages, or a radiotherapy which indirectly activates a pro-inflammatory signal on macrophages, when used with a SHP-1 inhibitor both demonstrated remarkable anti-tumor effects. See the Examples.

Exemplary pro-inflammatory agents include TLR agonists, STING activators, radiation therapies, PAMP/DAMP activators, checkpoint inhibitors, pro-inflammatory cytokines or chemokines, chemotherapies, bacteria components, cancer vaccines, and oncolytic viruses. Other exemplary pro-inflammatory agents include sound treatments (e.g., high intensity focused ultrasound), magnetic therapies, electrical treatments, and electrostatic treatments that can kill cancer cells. See e.g., Naud et al., Nanoscale Adv., 2020, 2, 3632-3655; Rominiyi et al., Br J Cancer. 2021 February; 124(4):697-709; Zandi et al., Cancer Med. 2021 November; 10(21): 7475-7491.

In some embodiments, the pro-inflammatory agent comprises an agent selected from the group consisting of TLR agonists, STING activators, radiation therapies, PAMP/DAMP activators, checkpoint inhibitors, pro-inflammatory cytokines or chemokines, chemotherapies, bacteria components, cancer vaccines, oncolytic viruses, sound treatments (e.g., high intensity focused ultrasound), magnetic therapies, electrical treatments, and electrostatic treatments.

In some embodiments, the pro-inflammatory agent comprises an agent selected from the group consisting of TLR agonists, STING activators, PAMP/DAMP activators, pro-inflammatory cytokines or chemokines, bacteria components, cancer vaccines, sound treatments (e.g., high intensity focused ultrasound), magnetic therapies, electrical treatments, and an electrostatic treatment.

In some embodiments, the pro-inflammatory agent is a sound treatment (e.g., high intensity focused ultrasound (HIFU), e.g., low intensity focused ultrasound (LIPUS)). See e.g., Wood et al., Ultrasound Med Biol. 2015 April; 41(4): 905-928; Sengupta et al., J Adv Res. 2018 November; 14: 97-111.

In some embodiments, the pro-inflammatory agent is a magnetic therapy (e.g., pulsed magnetic field, e.g., static magnetic field). See e.g., Tatarov et al., Comp Med. 2011 August; 61(4): 339-345; Sengupta et al., J Adv Res. 2018 November; 14: 97-111.

In some embodiments, the pro-inflammatory agent is an electrical treatment or electrochemical treatment. See e.g., Ciria et al., Chin J Cancer Res. 2013 April; 25(2): 223-234; Das et al., Front Bioeng Biotechnol. 2021; 9: 795300.

In some embodiments, the pro-inflammatory agent is an electrostatic treatment. See e.g., Zandi et al., Cancer Med. 2021 November; 10(21): 7475-7491.

In some embodiments, the pro-inflammatory agent is a thermoacoustic treatment. See e.g., Wen et al., Theranostics. 2017; 7(7): 1976-1989.

In some embodiments, the pro-inflammatory agent comprises a microbe (e.g., a fragment or lysate of a microbe). Examples of microbe include bacteria, fungi, and viruses.

In some embodiments, the pro-inflammatory agent comprises a TLR agonist (e.g., R848) and a cytokine (e.g., IFN-gamma).

### TLR Agonists

In some embodiments, the pro-inflammatory agent comprises or is a TLR agonist.

TLRs play a vital role in activating immune responses. TLRs recognize conserved pathogen-associated molecular patterns (PAMPs) expressed on a wide array of microbes, as well as endogenous DAMPs released from stressed or dying cells. TLR1, -2, -4, -5, -6, and -10 are expressed on the cell surface, whereas TLR3, -7, -8, and -9 are situated on endosomal membranes within the cell. TLR1 and TLR2 can heterodimerize to recognize a variety of bacterial lipid structures and cell wall components, such as triacylated lipoproteins, lipoteichoic acid, and β-glucans. TLR2 also heterodimerizes with TLR6 to bind diacylated lipopeptides. Additionally, TLR2 can bind various endogenous DAMPs, such as HSPs, HMGB1, uric acid, fibronectin, and other extracellular matrix proteins. It has also been suggested that TLR1 and TLR6 can heterodimerize with TLR10; however, the TLR agonist recognized by this dimer remains to be identified. TLR3 recognizes viral dsRNA, as well as synthetic analogs of dsRNA, such as ligand Poly I:C. TLR4 binds LPS in complex with lipid A binding protein, CD14, and myeloid differentiation protein 2, MD2 as well as recognizing various DAMPs. Endogenous TLR4 ligands, which have been described, include β-defensin 2, fibronectin extra domain A EDA, HMGB1, Snapin, and tenascin C. TLR5 recognizes bacterial flagellin, TLR7 and TLR8 bind viral ssRNA, whereas TLR9 interacts with unmethylated CpG DNA from bacteria and some viruses. Additional TLRs have been identified more recently in mice based on sequence homology of the highly conserved TIR domain. TLR10 is a surface receptor whose natural ligand remains unknown. TLR11, -12, and -13 are present in mice but not in humans. TLR11 was shown to bind a T. gondii profilin and uropathogenic Escherichia coli. The ligand for TLR12 has not yet been identified, whereas TLR13 is an endosomal receptor that recognizes VSV. See e.g., Kaczanowska et al., J Leukoc Biol. 2013 June; 93(6):847-63.

TLR signaling can act as a double-edged sword in cancer. It was found that TLR stimulation of cancer cells can lead to either tumor progression or inhibition. For example, Stimulation of TLR 2, 4, and 7/8 was found to lead to tumor progression via production of immunosuppressive cytokines, increased cell proliferation and resistance to apoptosis. R848-stimulation of TLR7/8 overexpressing pancreatic cancer cell line resulted in increased cell proliferation and reduced chemosensitivity. On the other hand, stimulation of TLR 2, 3, 4, 5, 7/8, and 9, often combined with chemo- or immunotherapy, can lead to tumor inhibition via different pathways. See e.g., Grimmig et al., Int J Oncol. (2015) 47:857-66; Urban-Wojciuk et al., Front Immunol. 2019; 10: 2388.

In some embodiments, the TLR agonist activates any of the TLRs.

In some embodiments, the TLR agonist activates TLR1 or TLR2, optionally wherein the TLR agonist comprises a triacylated lipoprotein, a peptidoglycan, zymosan, and/or Pam₃CSK₄.

In some embodiments, the TLR agonist activates any one of TLR2, TLR3, TLR4, TLR5, and TLR6, optionally wherein the TLR agonist comprises a diacylated lipopeptide, a hot shock protein, HMGB1, uric acid, fibronectin, and/or ECM protein.

In some embodiments, the TLR agonist activates TLR2, optionally wherein the TLR agonist comprises Pam3Cys, SMP-105, and/or CBLB612.

In some embodiments, the TLR agonist activates TLR3, optionally wherein the TLR agonist comprises dsRNA, Poly I:C, PolyICIC, Poly-IC12U, IPH302, ARNAX, and/or MPLA.

In some embodiments, the TLR agonist activates TLR4, optionally wherein the TLR agonist comprises LPS, lipoteichoic acid beta-defensin 2, fibronectin EDA, HMGB1, snapin, tenascin C, OK-432, AS04, and/or GLA-SE.

In some embodiments, the TLR agonist activates TLR5, optionally wherein the TLR agonist comprises flagellin, CBLB502, and/or M-VM3.

In some embodiments, the TLR agonist activates TLR6.

In some embodiments, the TLR agonist activates TLR7 or TLR8, optionally wherein the TLR agonist comprises ssRNA, CpG-A, poly G10, and/or poly G3.

In some embodiments, the TLR agonist activates TLR7, optionally wherein the TLR agonist comprises bistriazolyl and/or R848.

In some embodiments, the TLR agonist activates TLR8, optionally wherein the TLR agonist comprises VTX1463 and/or R848.

In some embodiments, the TLR agonist activates TLR9, optionally wherein the TLR agonist comprises unmethylated CpG DNA, CpG (e.g., CpG-7909, KSK-CpG, CpG-1826), MGN1703, dsSLIM, IM02055, SD101, and/or ODN M362.

In some embodiments, the TLR agonist activates TLR10, optionally wherein the TLR agonist comprises Pam₃CSK₄.

In some embodiments, the TLR agonist activates TLR11, optionally wherein the TLR agonist comprises Toxoplasma gondii profilin.

In some embodiments, the TLR agonist activates TLR12.

In some embodiments, the TLR agonist activates TLR13, optionally wherein the TLR agonist comprises VSV.

In some embodiments, the TLR agonist activates a TLR on a macrophage.

In some embodiments, the TLR agonist activates TLR1, TLR2, TLR3, TLR4, TLR7, TLR8, and/or TLR9.

In some embodiments, the TLR comprises TLR1, TLR4, and/or TLR9. In some embodiments, the TLR comprises TLR9.

In some embodiments, the TLR comprises TLR2, TLR4, TLR7, and/or TLR8.

In some embodiments, the TLR agonist comprises CpG. In some embodiments, the TLR agonist comprises polyL:C. In some embodiments, the TLR agonist comprises CpG and/or polyL:C. In some embodiments, the TLR agonist comprises CpG, polyL:C and/or R848.

In some embodiments, the TLR agonist is R848, 3M-852A, Motolimod, Bropirimine or Vesatolimod. In some embodiments, the TLR agonist is R848.

In some embodiments, the method described herein further comprises assessing whether the individual has an ongoing infection. In some embodiments, a reduced amount of the TLR agonist is administered when the individual has an ongoing infection. In some embodiments, the administration of TLR agonist can be avoided when the individual has an ongoing infection.

### Radiation Therapy

In some embodiments, the pro-inflammatory agent comprises or is a radiation therapy. Radiation activates the interconnected network of cytokines, adhesion molecule, ROS/RNS and DAMPs leading to a self-amplified cascade, which generates pro-inflammatory, pro-oxidant tumor microenvironment and ultimately tumor cell death. See e.g., McKelvey et al., Mamm Genome. 2018; 29(11): 843-865.

In some embodiments, the radiation therapy comprises irradiation at site of the cancer to be treated.

In some embodiments, the radiation therapy comprises irradiation at a site that is different from the site of the cancer to be treated.

In some embodiments, the radiation therapy is intraoperative radiation therapy (“IORT”). In particular embodiments, the radiation is localized to a tumor site. The patient may be subjected to intraoperative radiation prior to resection of the tumor or following resection of the tumor. The tumor site may comprise different types of cells including cancerous and benign cells. In certain embodiments, the radiation therapy is stereotactic body radiotherapy (“SBRT”) or stereotactic radiosurgery (“SRS”).

In some embodiments, the radiation is ionizing radiation such as particle beam radiation. The particle beam radiation may be selected from any of electrons, protons, neutrons, heavy ions such as carbon ions, or pions. The ionizing radiation may be selected from x-rays, UV-light, 7-rays, or microwaves. In some embodiments, the radiation therapy may comprise subjecting the patient to one or more types of radiation therapy.

In some embodiments, a radio sensitizer is used to sensitize the tumor cells to radiation. The use of such pharmaceuticals, called radiosensitizers, provides a method of increasing the radiosensitivity of tumors to radiation therapy, avoiding the need to increase radiation dosages to levels that are harmful to surrounding organs and tissues. See e.g., U.S. Pat. No. 9,656,098B2.

In some embodiments, the dose of the radiation therapy is non-ablative, insufficient to eliminate the tumor (kill all tumor cells). In some embodiments, the radiation therapy is selected from the group consisting of external-beam radiation therapy, internal radiation therapy (brachytherapy), intraoperative radiation therapy (IORT), systemic radiation therapy, radioimmunotherapy, and administration of radiosensitizers and radioprotectors.

In some embodiments, the radiation therapy is external-beam radiation therapy, optionally comprising three-dimensional conformal radiation therapy (3D-RT), intensity modulated radiation therapy (IMRT), photon beam therapy, image-guided radiation therapy (IGRT), and sterotactic radiation therapy (SRT).

In some embodiments, the radiation therapy comprises administering a radiopharmaceutical. The radiopharmaceuticals can be delivered via any vehicle such as a cell, a protein, or a small molecule complex. In some embodiments, the radiopharmaceutical is administered to the tumor tissue. See e.g., Sgouros et al. Radiopharmaceutical therapy in cancer: clinical advances and challenges. Nat Rev Drug Discov 19, 589-608 (2020).

In some embodiments, the radiation therapy is brachytherapy, optionally comprising interstitial brachytherapy, intracavitary brachytherapy, intraluminal radiation therapy, and radioactively tagged molecules given intravenously.

### STING Activator

In some embodiments, the pro-inflammatory agent comprises or is a STING activator.

Stimulator of IFN genes (STING, also known as TMEM173, MITA, MPYS or ERIS) is a pattern recognition receptor (PRR) that recognizes cytosolic DNA in the form of cyclic dinucleotides (CDNs), such as the bacterial product cyclic-guanosine monophosphate-adenosine monophosphate (3′3′ cGAMP). In addition to bacterial components, other forms of DNA from viruses, or the host cell, that find their way into the cytosol are recognized by an enzyme c-GMP-AMP (cGAMP) synthase (cGAS). Upon cytosolic DNA binding, cGAS converts ATP and GTP into the metazoan-specific CDN 2′3′-cGAMP for STING recognition and activation. STING is a transmembrane protein that exists as dimers anchored within the endoplasmic reticulum membrane and forms a V-shaped pocket that enables cytosolic CDN binding. Ligand binding results in significant conformational changes in the C-terminal domain of STING, mediating its transport to Golgi compartments. At the Golgi, STING recruits TANK-binding kinase 1 (TBK1), which facilitates IRF3 phosphorylation, nuclear translocation and the strong induction of transcription of type I IFNs (e.g., IFN-β). STING also triggers a robust pro-inflammatory cytokine response [e.g., tumor necrosis factor (TNF)] by activating Nuclear Factor-kappa B (NF-κB) and this part of the pathway can be mediated independent of TBK1 via a closely related homologue protein, IKKϵ. See e.g., Peng et al., Front Immunol. 2022 Feb. 25; 13:794776; Amougezar et al., Cancers (Basel). 2021 May 30; 13(11):2695.

In some embodiments, the STING activator is a cyclic-guanosine monophosphate-adenosine monophosphate (cGAMP, e.g., 3′3′ cGAMP, e.g., 2′3′ cGAMP).

In some embodiments, the STING activator is a bacterial vector (e.g., SYNB1891, STACT-TREX-1).

In some embodiments, the STING activator is a CDN compounds (e.g., ADU-S100, BI-STING, BMS-986301, GSK532, JNJ-4412, MK-1454, SB11285, 3′3′-cyclic AIMP).

In some embodiments, the STING activator is a non-CDN small molecule (e.g., ALG-031048, E7755, JNJ-'6196, MK-2118, MSA-1, MSA-2, SNX281, SR-717, TAK676, TTI-10001).

In some embodiments, the STING activator is a nanovaccine (e.g., PC7A NP, cCAMP-NP, ONM-500).

In some embodiments, the STING activator is an antibody-drug conjugate (e.g., XMT-2056, CRD-5500).

Other exemplary STING activators can be found in Amougezar et al., Cancers (Basel). 2021 May 30; 13(11):2695, which is incorporated by reference here by its entirety.

### PAMP/DAMP Activators

In some embodiments, the pro-inflammatory agent comprises or is a PAMP/DAMP activator.

The organism senses microbial infection through innate receptors encoded in the genome, called pattern-recognition receptors, including the Toll-like receptors (TLRs), the nucleotide-binding and oligomerization domain (NOD)-like receptors, and retinoic acid-inducible gene I (RIG-I)-like receptors. These receptors recognize pathogen-associated molecular patterns (PAMPs) expressed by bacteria, fungi, and viruses, but also bind damage-associated molecular patterns (DAMPs), which are molecules released by sterile injury. Thus, PAMPs and DAMPs that bind to the same type of receptors initiate identical intracellular pathways terminating in identical effector functions. See e.g., Alisi et al., Hepatology. 2011 November; 54(5):1500-2.

In some embodiments, the pro-inflammatory agent is a PAMP activator. Exemplary PAMP activator includes triacyl lipopeptides, LPS, lipoprotein, peptidoglycan, zymosan, lipoteichoic acid, trypanosomal phospholipids, Pam3Cys porins, lipoarabinomannan, double-stranded RNA, poly(I:C), trepanosomal lipids, taxol, Pseudomonas exoenzyme S, RSV F protein, MMTV envelope protein, flagellin, diacyl lipopeptides, single-stranded RNA, imiquimod, single-stranded RNA, resquimod, bacterial/viral DNA, CpG DNA, ureobacteria, and toxoplasma LPS.

In some embodiments, the pro-inflammatory agent is a DAMP activator. Exemplary DAMP activator includes defensins, HSP60, HSP70, messenger RNA, low-molecular-weight hyaluronic acid, fibrinogen, fibronectin, fx1-defensin, heparan sulfate, HSP60, HSP70, HSP90, HMGB1, and unmethylated CpG DNA.

### Chemotherapeutic Agent

In some embodiments, the pro-inflammatory agent comprises or is a chemotherapeutic agent.

In some embodiments, the chemotherapeutic agent is an alkylating agent. Exemplary alkylating agents include nitrogen mustard (e.g., endamustine, cyclophosphamide, ifosfamide), nitrosoureas (e.g., carmustine, lomustine), platinum analogs (e.g., carboplatin, cisplatin, oxaliplatin), triazenes (e.g., dacarbazine, procarbazine, temozolamide), alkyl sulfonate (e.g., busulfan), and ethyleneimine (e.g., thiotepa).

In some embodiments, the chemotherapeutic agent is an antimetabolite. Exemplary antimetabolites include cytidine analogs (e.g., azacitidine, decitabine, cytarabine, gemcitabine), folate antagonists (e.g., methotrexate, pemetrexed), purine analogs (e.g., cladribine, clofarabine, nelarabine), pyrimidine analogs (e.g., fluorouracil (5-FU), capecitabine (prodrug of 5-FU)).

In some embodiments, the chemotherapeutic agent is an antimicrotubular agent. Exemplary antimmicrotubular agents include topoisomerase II inhibitors (e.g., anthracyclines, doxorubicin, daunorubicin, idarubicin, mitoxantrone), topoisomerase I inhibitors (e.g., irinotecan, topotecan), taxanes (e.g., paclitaxel, docetaxel, cabazitaxel), vinca alkaloids (e.g., vinblastine, vincristine, vinorelbine), antibiotics (e.g., actinomycin D, bleomycin, daunomycin).

Other exemplary chemotherapeutic agents include hydroxyurea, tretinoin, arsenic trioxide, and proteasome inhibitors (e.g., bortezomib).

### Pro-Inflammatory Cytokines

In some embodiments, the pro-inflammatory agent is a pro-inflammatory cytokine.

In some embodiments, the pro-inflammatory cytokine promotes the M1 macrophages. See e.g., Duque et al., Front Immunol. 2014; 5: 491. In some embodiments, the pro-inflammatory cytokine comprises or is TNF, IFNγ, and/or GM-CSF.

In some embodiments, the pro-inflammatory cytokine comprises IL-6, TNFα, a cytokine from IL-1 family (e.g., IL-1α, IL-1β, IL-18, IL-33 and IL-36), and/or IFNγ.

In some embodiments, the pro-inflammatory cytokine comprises a cytokine from IL-1 family. In some embodiments, the pro-inflammatory cytokine comprises any one or more of IL-1α, IL-1β, IL-18, IL-33, and IL-36. See e.g., Sims, J., Smith, D. The IL-1 family: regulators of immunity. Nat Rev Immunol 10, 89-102 (2010).

### Checkpoint Inhibitors

In some embodiments, the pro-inflammatory agent is a checkpoint inhibitor. Immune checkpoints are pathways with inhibitory or stimulatory features that maintain self-tolerance and assist with immune response. The most well-described checkpoints are inhibitory in nature and include the cytotoxic T lymphocyte-associated molecule-4 (CTLA-4), programmed cell death receptor-1 (PD-1), and programmed cell death ligand-1 (PD-L1). See e.g., Marin-Acevedo et al., J Hematol Oncol 14, 45 (2021).

In some embodiments, the checkpoint inhibitor targets CLTA-4, PD-1 or PD-L1 (e.g., an antibody targeting CTLA-4, PD-1 or PD-L1).

In some embodiments, the checkpoint inhibitor targets LAG-3, TIM-3, B7-H3, B7-H4, A2aR, CD73, NKG2A, PVRIG/PVRL2, CEACAM1, CEACAM 5/6, FAK, CCL2/CCR2, LIF, CD47/SIRPα, CSF-1(M-CSF)/CSF-1R, IL-1/IL-1R3 (IL-1RAP), IL-8, SEMA4D, Ang-2, CLEVER-1, Axl, or phosphatidylserine.

In some embodiments, the checkpoint inhibitor comprises or is lipilimumab, Cemiplimab, Nivolumab, Pembrolizumab, Atezolizumab, Avelumab, Durvalumab, LAG525 (IMP701), REGN3767, BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lanotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), or Bavituximab.

### Cancer Vaccine

In some embodiments, the pro-inflammatory agent comprises or is a cancer vaccine. Cancer vaccine stimulates anti-tumor immunity with tumor antigens, which could be delivered in the form of whole cells, peptides, nucleic acids, etc. Ideal cancer vaccines could overcome the immune suppression in tumors and induce both humoral immunity and cellular immunity.

In some embodiments, the cancer vaccine comprises a cell-based vaccine, a peptide-based vaccine, a viral-based vaccine, and/or a nucleic acid-based vaccine. See e.g., Liu et al., J Hematol Oncol 15, 28 (2022).

Cell-based vaccines are the form of cancer vaccines initially. Cell-based cancer vaccines are often prepared from whole cells or cell fragments, containing almost tumor antigens, inducing a broader antigen immune response. DC vaccine is an important branch of cell-based vaccines. Personalized neoantigen cancer vaccines based on DC have shown promising anti-tumor effects in clinical. Viruses are naturally immunogenic and their genetic material can be engineered to contain sequences encoding tumor antigens. Several recombinant viruses, such as adenovirus, can infect immune cells as vectors. The engineered virus vaccines can present tumor antigens in large quantities in the immune system and produce anti-tumor immunity. Furthermore, the oncolytic virus can be used as a vector as well. Except for providing tumor antigens, the virus itself can also lyse the tumor, release tumor antigens, further increase the vaccine's effectiveness, and produce long-term immune memory.

Peptide-based subunit vaccines, including chemical and biosynthetic preparations of predicted or known specific tumor antigens, induce a robust immune response against the particular tumor antigen site. Peptide-based subunit vaccine combined with adjuvants can efficiently provoke humoral immune response, suitable for preventing and treating viral infectious diseases.

HBV and HPV vaccines for liver and cervical cancers were primarily peptide-based subunit vaccines. Especially, virus-like particles (VLP)-based subunit vaccines that can activate cellular immune responses have shown good anti-tumor activity in recent years.

The nucleic acid vaccine induces strong MHC I mediated CD8+ T cell responses; thus, it is a desirable cancer vaccine platform [63]. Nucleic acid vaccines can simultaneously deliver multiple antigens to trigger humoral and cellular immunity. Additionally, nucleic acid vaccines can encode full-length tumor antigens, allowing APC to cross-present various epitopes or present several antigens simultaneously. Finally, the nucleic acid vaccine preparation is simple and fast, which is suitable for developing personalized neoantigen cancer vaccines.

### Oncolytic Virus

In some embodiments, the pro-inflammatory agent is an oncolytic virus (OV). The oncolytic viruses (OVs) are organisms able to identify, infect, and lyse different cells in the tumor environment, aiming to stabilize and decrease the tumor progression. They can present a natural tropism to the cancer cells or be oriented genetically to identify specific targets. See e.g., Apolonio et al., World J Virol. 2021 Sep. 25; 10(5): 229-255.

Oncolytic viruses represent an exciting new avenue of cancer therapy. Such viruses have the remarkable ability to hunt and terminate cancer cells while leaving healthy cells unharmed, as well as enhancing the immune system's ability to recognize and terminate cancer cells. See e.g., Cancer Cell. 2022 Aug. 15; S1535-6108(22)00357-9.

In some embodiments, the oncolytic virus comprises or is an adenovirus (e.g., ONYX-15, LOAd703 virus), a protoparvovirus, a parvovirus (e.g., H-1PV), a vaccinia virus (VACV), a Reovirus (e.g., Reolysin), or a Herpes simplex virus (HSV, e.g., HSV-1, HSV-2, G207, L1BR1, HF10, T-VEC, Orien X010).

Other exemplary oncolytic viruses include JX-593, Coxsackievirus A21 (CVA21), marabi virus or its MG1 variant, DNX2440 adenovirus, fowl pox virus, and Sendai virus.

### Cells

In some embodiments, the pro-inflammatory agent comprises cells that that trigger inflammatory factors. In some embodiments, the cells are tumor-infiltrating lymphocytes. In some embodiments, the cells specifically recognize a tumor antigen (e.g., being engineered to express a CAR recognizing a tumor antigen). In some embodiments, the cells are T cells. In some embodiments, the cells are CAR-T cells. In some embodiments, the cells are NK cells (e.g., CAR-NK cells). In some embodiments, the cells are neutrophils (e.g., CAR-expressing neutrophils cells). In some embodiments, the cells are TCR-T cells. In some embodiments, the cells are APCs (e.g., macrophages or dendritic cells). In some embodiments, the cells are CAR-macrophages or CAR-monocytes. In some embodiments, the cells are SIRPαnt-macrophages. In some embodiments, the cells are stem cells. In some embodiments, the cells are allogenic. In some embodiments, the cells are autologous.

### Immune Cells, Monocytes or Macrophages

Immune cells described herein encompass various kinds of immune cells.

In some embodiments, the immune cells comprise monocytes or macrophages described herein. In some embodiments, the macrophages are identified by F4/80 expression. In some embodiments, the macrophages have a M1 phenotype. In some embodiments, at least 50%, 60%, 70%, 80%, 90%, 95%, 98%, or 99%) of the macrophages in the immune cells have a M1 phenotype.

In some embodiments, the macrophages are engineered to be deficient in SHP-1 expression and/or activation. In some embodiments, the monocytes or macrophages express a reduced level of SHP-1 for at least a period of time (e.g., for at least 1, 2, 3, 4, or 5 days) or are resistant to activation for at least a period of time (e.g., for at least 1, 2, 3, 4, or 5 days). In some embodiments, the period of time is no more than about 10, 9, 8, 7, 6, 5, 4, or 3 days.

In some embodiments, the monocytes or macrophages have reduced SHP-1 activity for no more than about 5 consecutive days (e.g., for no more than 5, 4, or 3 days) before the SHP-1 activity level returns to normal.

Methods to engineer monocytes or macrophages to transiently express a reduced level of SHP-1 are well-known in the field. Exemplary methods include contacting the monocytes or macrophages with a SHP-1 inhibitor described herein (such as a small molecule, a nucleic acid (e.g., a siRNA, a shRNA, an antisense RNA, a microRNA), a nucleic acid editing system (e.g., a CRISPR system), and a protein agent (e.g., an antibody agent that targets SHP-1 or activated SHP-1)) in vivo or in vitro.

In some embodiments, the immune cells comprise T cells (e.g., CAR-T cells).

In some embodiments, the immune cells comprise NK cells (e.g., CAR-NK cells).

In some embodiments, the immune cells comprise neutrophils (e.g., CAR-expressing neutrophils cells).

In some embodiments, the immune cells comprise antigen presenting cells (APCs, e.g., dendritic cells).

In some embodiments, the immune cells are derived from the same individual (i.e., autologous). In some embodiments, the immune cells are allogeneic.

In some embodiments, the immune cells are engineered to express a chimeric antigen receptor, optionally wherein the chimeric antigen receptor specifically binds to a tumor antigen.

In some embodiments, the immune cells express a high level of MHC-I, MHC-II, CD80 and/or CD86. In some embodiments, the immune cells express a high level of MHC-I, MHC-II, CD80 and/or CD86 when the expression level of MHC-I, MHC-II, CD80 and/or CD86 on the immune cells is comparable (e.g., at least more than 50%) of that on activated antigen presenting cells (APCs).

In some embodiments, the immune cells express one or more pro-inflammatory cytokines, optionally wherein the one or more pro-inflammatory cytokines comprise TNFα and/or IL-12.

In some embodiments, the immune cells do not express a significant level of TGFβ and/or IL-10.

In some embodiments, the SHP-1 inhibitor and the immune cells are administered within 24 hours (e.g., 12 hours, 8 hours, 4 hours, 2 hours, 1 hour, or 0.5 hour) of each other, optionally wherein the SHP-1 inhibitor and the immune cells are administered within 4 hours of each other.

In some embodiments, the SHP-1 inhibitor, the immune cells, and a pro-inflammatory agent described above are administered within 24 hours (e.g., 12 hours, 8 hours, 4 hours, 2 hours, 1 hour, or 0.5 hour) of each other. In some embodiments, the immune cells are administered simultaneously or concurrently with the SHP-1 inhibitor and/or the pro-inflammatory agent.

### Inflammation Reaction or Ongoing Infection

There has been a body of evidence that both acute and chronic inflammation are associated with the development and progression of cancer. Progress in research on inflammation revealed a connection between inflammatory processes and neoplastic transformation, the progression of tumor, and the development of metastases and recurrences. Moreover, the tumor invasive procedures (both surgery and biopsy) affect the remaining tumor cells by increasing their survival, proliferation and migration. One of the concepts explaining this phenomena is an induction of a wound healing response. While in normal tissue it is necessary for tissue repair, in tumor tissue, induction of adaptive and innate immune response related to wound healing, stimulates tumor cell survival, angiogenesis and extravasation of circulating tumor cells. See e.g., Singh et al., Ann Afr Med. 2019 July-September; 18(3): 121-126; Piotrowski et al., Rep Pract Oncol Radiother. 2020 May-June; 25(3):422-427.

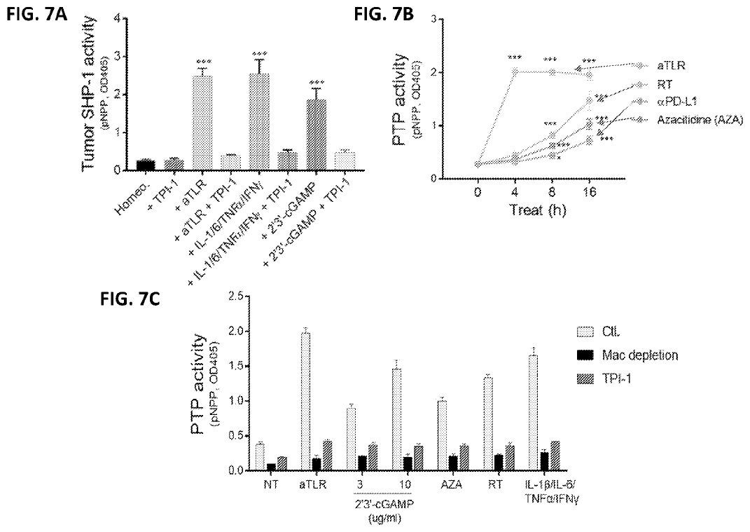

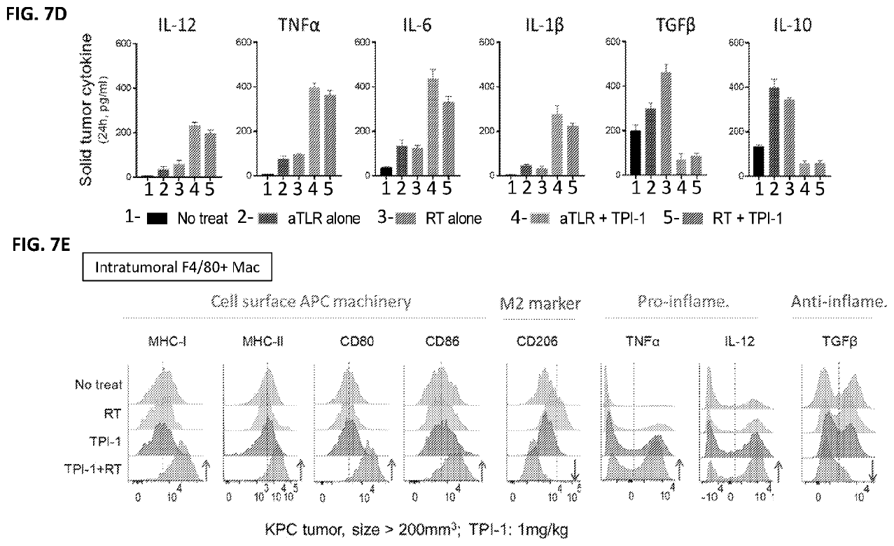

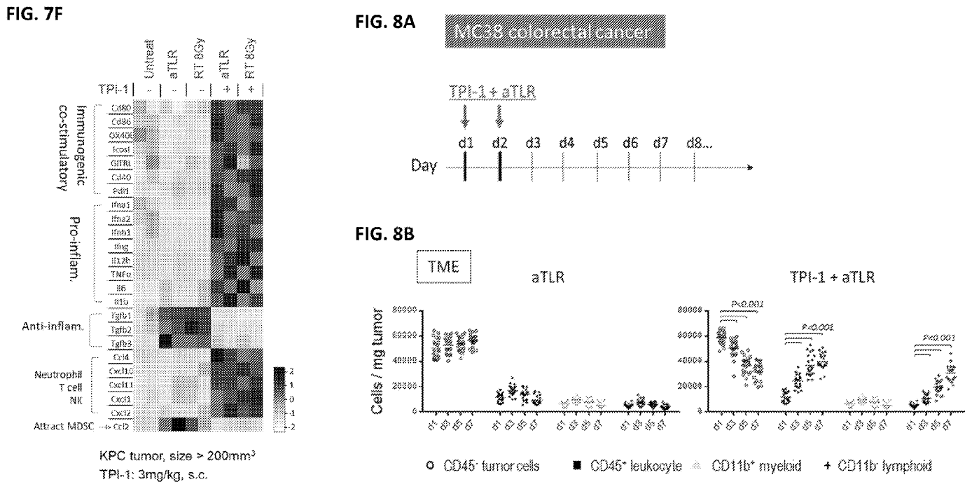

However, as demonstrated in this application, a combined use of a SHP-1 inhibitor and a pro-inflammatory agent unleashes proinflammatory response and transforms an immunosuppressive tumor environment into a place with an inflammation signature. See e.g., FIG. 7F. Remarkable anti-tumor effects were achieved. These results provide support for using methods described herein for treating an individual who is in under an inflammation reaction.

In some embodiments, the individual is under an inflammation reaction or has an ongoing infection when being treated with the methods described herein. The inflammation reaction described herein can be reflected by, e.g., a) an increase in one or more (e.g., at least one, two, three, four, five) inflammatory cytokines (such as IFNγ, IL-12b, TNFα, IL-6, IL-1b, IFN-a1, IFN-a2, IFN-bl), b) a decrease in one or more (e.g., at least one, two or three) anti-inflammatory cytokine (such as TGFb1, TGFb2, TGFb3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages).

In some embodiments, the inflammation reaction is an acute inflammation reaction.

In some embodiments, the inflammation reaction is in the tumor. In some embodiments, the inflammation reaction is at a site distinct from the tumor.

In some embodiments, there is an inflammation reaction where there are at least two (e.g., two, three, four or five events) selected from the group consisting of a) an increase in one or more (e.g., at least one, two, three, four, five) inflammatory cytokines (such as IFNγ, IL-12b, TNFα, IL-6, IL-1b, IFN-a1, IFN-a2, IFN-b1), b) a decrease in one or more (e.g., at least one, two or three) anti-inflammatory cytokine (such as TGFb1, TGFb2, TGFb3), c) an increase in the infiltrating immune cells (such as T cells, NK cells, macrophages, neutrophils), d) a decrease in suppressive immune cells (such as MDSCs), and/or e) an increase in one or more (e.g., at least one, two, three, four, or five) immunogenic co-stimulatory molecules (such as CD80, CD86, OX40L, CD40, ICOS-L, PD-L1, GITRL) in the tissue (e.g., tumor tissue) or immune cells (such as macrophages).

In some embodiments, the increase described herein refers to at least about 20%, 30%, 40%, 50%, 60%, 70%, 80%, 90%, 100%, 125%, 150%, 175%, or 200% more in the amount of level as compared to that in a reference state, optionally wherein the reference state is when the individual is neither treated with the methods described herein nor infected by a pathogen. In some embodiments, the increase described herein refers to at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more in the amount of level as compared to that in a reference state, optionally wherein the reference state is when the individual is neither treated with the methods described herein nor infected by a pathogen. In some embodiments, the reference state is when a healthy individual is not infected by a pathogen.

In some embodiments, the decrease described herein refers to at least about 10%, 20%, 30%, 40%, 50%, 60%, 70%, 80%, 90%, 95%, 98%, 99%, or 99.9% less in the amount of level as compared to that in a reference state, optionally wherein the reference state is when the individual is neither treated with the methods described herein nor infected by a pathogen. In some embodiments, the reference state is when a healthy individual is not infected by a pathogen.

In some embodiments, the individual has an inflammation reaction (e.g., in the tumor, e.g., in a site distinct from the tumor) within about one week, 6 days, 5 days, 4 days, 3 days, 2 days, or one day prior to and/or after the administration of the SHP-1 inhibitor.

In some embodiments, the individual has an ongoing inflammation reaction (e.g., in the tumor, e.g., in a site distinct from the tumor) when the SHP-1 inhibitor is administered.

In some embodiments, the individual has an ongoing infection when the SHP-1 inhibitor is administered. In some embodiments, the method further comprises assessing the presence of an infection in the individual, e.g., an infection associated with a virus, a fungus, and/or a bacteria.

In some embodiments, the individual has an ongoing infection (e.g., a bacteria infection, a virus infection, a fungus infection) and the method further comprises administering an antibacterial therapy (e.g. an antibiotic). an antiviral therapy, an anti-microbial therapy or an anti-protozoan therapy.

### Immunogenic Cell Death

In some embodiments, the individual has immunogenic cell death when being treated with the methods described herein.

Immunogenic cell death (ICD) is a type of cancer cell death that can be induced by different stressors, including but not limited to (1) intracellular pathogens; (2) conventional chemotherapeutics such as anthracyclines, DNA-damaging agents, and proteasomal inhibitors; (3) targeted anticancer agents such as the tyrosine kinase inhibitor crizotinib, the epidermal growth factor receptor-specific monoclonal antibody cetuximab and poly-ADP-ribose polymerase (PARP) inhibitors; and (4) numerous physical modalities, encompassing hypericin- and redaporfin-based photodynamic therapy, extracorporeal photochemotherapy, various forms of ionizing radiation, high hydrostatic pressure, and severe heat shock. It involves the activation of the immune system against cancer in immunocompetent hosts. ICD comprises the release of damage-associated molecular patterns (DAMPs) from dying tumor cells that result in the activation of tumor-specific immune responses, thus eliciting long-term efficacy of anticancer drugs by combining direct cancer cell killing and antitumor immunity. DAMPs include the cell surface exposure of calreticulin (CRT) and heat-shock proteins (HSP70 and HSP90), extracellular release of adenosine triphosphate (ATP), high-mobility group box-1 (HMGB1), type I IFNs and members of the IL-1 cytokine family. See e.g., Ahmed et al., Mol Oncol. 2020 December; 14(12):2994-3006 and Fucikova et al., Cell Death Dis. 2020 Nov. 26; 11(11):1013.

Key DAMPs for cell death to be perceived as immunogenic include calreticulin, high-mobility group box 1 (HMGB1), ATP, annexin A1 (ANXA1), and type I IFN. The main hallmarks of immunogenic cell death (ICD) can be assessed by flow cytometry, (immuno)fluorescence microscopy, immunoblotting, or luminometry, based on a variety of different approaches. See e.g., Cell Death Dis. 2020 Nov. 26; 11(11):1013.

In some embodiments, the individual has ICD (e.g., in the tumor, e.g., in a site distinct from the tumor) within about one week, 6 days, 5 days, 4 days, 3 days, 2 days, or one day prior to and/or after the administration of the SHP-1 inhibitor.

In some embodiments, the individual has ongoing ICD (e.g., in the tumor, e.g., in a site distinct from the tumor) when the SHP-1 inhibitor is administered.

In some embodiments, the individual has ICD when a sample from the cancer has a higher level of one or more (e.g., at least 10%, 20%, 30%, 40%, 50%, 60%, 70%, 80%, 90% more) DAMPs than a reference sample (e.g., a corresponding sample in a healthy control, e.g., a sample from the cancer prior to the administration of a therapy that induces ICD. In some embodiments, the DAMPs are selected from the group consisting of endoplasmic reticulum (ER) chaperones (e.g., calreticulin (CALR), e.g., heat-shock proteins (HSPs)), the non-histone chromatin-binding protein high-mobility group box 1 (HMGB1), the cytoplasmic protein annexin A1 (ANXA1), and the small metabolite ATP, and type I interferons (IFNs).

### Individuals

In some embodiments, the individual has a solid tumor. In some embodiments, the individual has a hematologic cancer.

In some embodiments, the individual has an advanced cancer. In some embodiments, the individual has a late stage cancer. In some embodiments, the individual has a malignant cancer. In some embodiments, the individual has a cancer that is in stage II, III or IV. In some embodiments, the individual has an inoperable tumor and/or metastases. In some embodiments, the individual is a terminally ill individual.

In some embodiments, the individual has been subjected (e.g., within 1, 2, 4, 8, 12, 16, 20, or 24 hours, e.g., within 1, 2, 3, 4, 5, 6 or 7 days before the administration of the SHP-1 inhibitor) to a therapy that induces an inflammation reaction or an immunogenic cell death (e.g., radiotherapy). In some embodiments, the individual is to be subjected to (e.g., within 1, 2, 4, 8, 12, 16, 20, or 24 hours, e.g., within 1, 2, 3, 4, 5, 6 or 7 days after the administration of the SHP-1 inhibitor) a therapy that induces an inflammation reaction or an immunogenic cell death (e.g., radiotherapy).

In some embodiments, the individual has been subjected (e.g., within 1, 2, 4, 8, 12, 16, 20, or 24 hours, e.g., within 1, 2, 3, 4, 5, 6 or 7 days before the administration of the SHP-1 inhibitor) to a pro-inflammatory agent (such as any of the pro-inflammatory agents described herein). In some embodiments, the individual is to be subjected to (e.g., within 1, 2, 4, 8, 12, 16, 20, or 24 hours, e.g., within 1, 2, 3, 4, 5, 6 or 7 days after the administration of the SHP-1 inhibitor) a pro-inflammatory agent (such as any of the pro-inflammatory agents described herein).

In some embodiments, the individual does not have an autoimmune disease.

In some embodiments, the individual is a female. In some embodiments, the individual is a male.

In some embodiments, the individual is a human. In some embodiments, the individual is at least about 50, 55, 60, 65, 70 or 75 years old.

In some embodiments, the individual is selected for treatment based upon a high expression level and/or a high activation level of SHP-1 in the tumor tissue. In some embodiments, the individual has a high expression level and/or a high activation level of SHP-1 when the expression level and/or the activation level is at least about 20%, 30%, 40%, 50%, 60%, 70%, 80%, 90%, 100%, 125%, 150%, 175%, or 200% more than a reference expression level and/or a reference activation level of SHP-1. In some embodiments, the individual has a high expression level and/or a high activation level of SHP-1 when the expression level and/or the activation level is at least about 5-fold, 10-fold, 20-fold, 30-fold, 40-fold, 50-fold, 60-fold, 70-fold, 80-fold, 90-fold, 100-fold, 150-fold, 200-fold, 250-fold, 500-fold, or 1000-fold more than a reference expression level and/or a reference activation level of SHP-1. In some embodiments, the reference expression level or the reference activation level of SHP-1 is the corresponding expression or activation level of SHP-1 in a reference state, wherein the individual is not treated with a pro-inflammatory agent (or any immune therapy).

In some embodiments, the individual is at risk of developing systemic inflammation and/or CRS. In some embodiments, the individual develops systemic inflammation and/or CRS prior to the administration of an agent that reduces systemic inflammation. Cytokine release syndrome can damage or cause organ failure in most organ systems. For example, organs that can become damaged due to CRS may include, but are not limited to, the lungs, the kidneys, the liver, the brain, the heart, the spleen, or any combination thereof, for example multi-organ failure.

In some embodiments, the individual is administered an agent that reduces systemic inflammation. In some embodiments, the administration occurs prior to the development of systemic inflammation in the individual. In some embodiments, the individual develops mild cytokine release syndrome. In some embodiments, the individual develops CRS of grade 1. Mild symptoms of CRS can include fever, fatigue, headache, rash, arthralgia, and myalgia. Mild CRS can be treated by treating the symptoms or by administration of anti-inflammatory drugs such as corticosteroids. Mild CRS can often be resolved within one to two weeks and does not require or necessitate hospitalization.

In some embodiments, the individual does not develop severe cytokine release syndrome. In some embodiments, the individual does not develop CRS of grade 2. In some embodiments, the individual does not develop CRS of grade 3. In some embodiments, the individual does not develop CRS of grade 4. More severe cases are characterized by hypotension and high fever, and severe CRS can progress to an uncontrolled systemic inflammatory response with vasopressor-requiring circulatory shock, vascular leakage, disseminated intravascular coagulation, and multi-organ system failure. More severe cases of CRS often require hospitalization of symptoms. Laboratory abnormalities that are common in patients with CRS include cytopenias, elevated creatinine and liver enzymes, deranged coagulation parameters, and a high CRP. There are four grading systems currently used for cytokine release syndrome, as shown in Table 1 below. See, e.g., Liu, D. and Zhao, J., J Hematol Oncol. 2018 Sep. 24; 11(1):121; and Shimabukuro-Vornhagen, A. et al., J Immunother Cancer. 2018 Jun. 15; 6(1):56, hereby incorporated by reference in their entirety.

In some embodiments, the individual has developed CRS prior to administration of an agent that reduces systemic inflammation. In some embodiments, the individual has developed CRS of grade 1. In some embodiments, the individual has developed CRS of grade 2. In some embodiments, the individual has developed CRS of grade 3. In some embodiments, the individual has developed CRS of grade 4. In some embodiments, the individual who has developed CRS is administered an agent that reduces systemic inflammation. In some embodiments, the agent that reduces systemic inflammation ameliorates, eliminates, or reverses the CRS, including organ damage, for example pro-inflammatory organ damage (e.g., nephritis, hepatitis, pneumonitis, myocarditis, appendicitis).

TABLE 1
Cytokine release syndrome medical grading systems.

| | Penn grading scale | CTCAE v4.0 | 2014 Lee et al. | MDACC |
|---|---|---|---|---|
| Grade | Mild reaction: treated with | Mild reaction; | Symptoms are | Temperature ≥38 |
| 1 | supportive care such as | infusion | not life- | °C (fever) or |
| | antipyretics and | interruption not | threatening and | grade 1 organ |
| | antiemetics | indicated; | require | toxicity |
| | | intervention not | symptomatic | |
| | | indicated | treatment only, | |
| | | | e.g., fever, | |
| | | | nausea, fatigue, | |
| | | | headache, | |
| | | | myalgias, and | |
| | | | malaise | |
| Grade | Moderate reaction: some | Therapy or | Symptoms | Systolic blood |
| 2 | signs of organ dysfunction | infusion | require and | pressure <90 |
| | (e.g., grade 2 creatinine or | interruption | respond to | mmHg |
| | grade 3 LFTs) related to | indicated but | moderate | (hypotension) but |
| | CRS and not attributable | responds | intervention. | responds to IV |
| | to any other condition. | promptly to | Oxygen | fluids or low- |
| | Hospitalization for | symptomatic | requirement <40% | dose vasopressors |
| | management of CRS- | treatment (e.g., | or hypotension | or needing |
| | related symptoms, | antihistamines, | responsive to | oxygen (FiO2 <40%) |
| | including fevers with | NSAIDs, | fluids or low dose | for SaO2 >90% |
| | associated neutropenia and | narcotics, IV | pressors or grade | (hypoxia) or |
| | need for IV therapies (not | fluids); | 2 organ toxicity | grade 2 organ |
| | including fluid | prophylactic | | toxicity |
| | resuscitation for | medications | | |
| | hypotension) | indicated for ≤24 | | |
| | | h | | |
| Grade | More severe reaction: | Prolonged | Symptoms | Systolic blood |
| 3 | hospitalization required | reaction (e.g., not | require and | pressure <90 |
| | for management of | rapidly | respond to | mmHg |
| | symptoms related to organ | responsive to | aggressive | (hypotension) and |
| | dysfunction, including | symptomatic | intervention. | needs high-dose |
| | grade 4 LFTs or grade 3 | medication and/or | Oxygen | or multiple |
| | creatinine related to CRS | brief interruption | requirement ≥40% | vasopressors or |
| | and not attributable to any | of infusion); | or hypotension | needing |
| | other conditions; this | recurrence of | requiring high- | oxygen(FiO2 ≥40%) |
| | excludes management of | symptoms | dose or multiple | for SaO2 >90% |
| | fever or myalgias; | following initial | pressors or grade | (hypoxia) or |
| | includes hypotension | improvement; | 3 organ toxicity | grade 3 organ |
| | treated with IV fluids | indicated for | or grade 4 | toxicity or grade |
| | (defined as multiple fluid | clinical sequelae | transaminitis | 4 transaminitis |
| | boluses for blood pressure | (e.g., renal | | |
| | support) or low-dose | impairment, | | |
| | vasopressors, | pulmonary | | |
| | coagulopathy requiring | infiltrates) | | |
| | fresh frozen plasma or | | | |
| | cryoprecipitate or | | | |
| | fibrinogen concentrate, | | | |
| | and hypoxia requiring | | | |
| | supplemental oxygen | | | |
| | (nasal cannula oxygen, | | | |
| | high-flow oxygen, CPAP, | | | |
| | or BiPAP). Patients | | | |
| | admitted for management | | | |
| | of suspected infection due | | | |
| | to fevers and/or | | | |
| | neutropenia may have | | | |
| | grade 2 CRS | | | |
| Grade | Life-threatening | Life-threatening | Life-threatening | Life-threatening |
| 4 | complications such as | consequences; | symptoms. | hypotension or |
| | hypotension requiring | pressor or | Requirements for | needing ventilator |
| | high dose vasopressors | ventilator support | ventilator support | support or grade |
| | and a hypoxia requiring | indicated | or grade 4 oxygen | 4 oxygen toxicity |
| | mechanical ventilation | | toxicity | (excluding |
| | | | (excluding | transaminitis) |
| | | | transaminitis) | |

In some embodiments, the individual does not develop cytokine storm. In some embodiments, the individual develops mild cytokine storm. In some embodiments, the individual does not develop severe or life-threatening cytokine storm. Cytokine storm appears to be mainly a result of non-specific T cell activation, whereas CRS is more often a direct consequence of antigen-specific T cell activation. The clinical manifestations of cytokine storm and CRS can be similar (Liu, D. and Zhao, J., J Hematol Oncol. 2018 Sep. 24; 11(1):121).

### Cancer

Cancer described here can be any type or kind. In some embodiments, the cancer is a solid tumor. In some embodiments, the cancer is a hematologic cancer.

In some embodiments, the cancer is an advanced cancer. In some embodiments, the cancer is a late stage cancer. In some embodiments, the cancer is a terminal cancer. In some embodiments, the cancer is in stage II, III or IV. In some embodiments, the cancer is an inoperable tumor and/or is malignant.

In some embodiments, the tumor is at least 0.2 cm, 0.4 cm, 0.6 cm, 0.8 cm, 1 cm, 2 cm, 3 cm, 4 cm or 5 cm in length.

Examples of cancers described herein include, but are not limited to, adrenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments, the cancer is a virus-infection-related cancer. In some embodiments, the cancer is a human papillomavirus (HPV)-related cancer (e.g., HPV-related cervical cancer, e.g., HPV-related head and neck cancer, e.g., HPV related squamous cell carcinoma). In some embodiments, the cancer is human herpes virus 8 (HHV8) related cancer (e.g., Kaposi sarcoma). In some embodiments, the cancer is human T-lymphotrophic virus (HTLV-1)-related cancer (e.g., adult T cell leukemia or lymphoma). In some embodiments, the cancer is Epstein-Barr virus (EBV) related cancer (e.g., Burkitt lymphoma, Hodgkin's and non-Hodgkin's lymphoma, stomach cancer). In some embodiments, the cancer is hepatitis B virus (HBV) related cancer (e.g., liver cancer). In some embodiments, the cancer is hepatitis C virus) related cancer (e.g., liver cancer, non-Hodgkin's lymphoma).

In some embodiments, the cancer is a liver cancer, a kidney cancer, an endometrial cancer, a thymic epithelial neoplasma, lung cancer, spindle cell sarcoma, chondrosarcoma, uterine smooth muscle, colon cancer, or pancreatic cancer.

In some embodiments, the cancer has been subjected to and/or failed one or more prior therapy (e.g., an immune checkpoint blockage therapy (e.g., a PD-1 antibody), a chemotherapy, a surgery, a cell therapy (e.g., an allogenic NK cell infusion therapy)).

In some embodiments, the cancer is a recurrent or refractory cancer.

In some embodiments, the cancer is refractory to one or more of irradiation therapy, chemotherapy, or immunotherapy (e.g., checkpoint blockade).

### Dosing, Method of Administration, and Delivery Vehicles

The SHP-1 inhibitor, the pro-inflammatory agent, and the immune cells (e.g., monocytes/macrophages) described herein can be administered at any desired dosage. Exemplary dosing regimens are described in e.g., “SHP-1 inhibitors” section.

In some aspects, the size of the dose in the pro-inflammatory agent, the SHP-1 inhibitor and/or the immune cells (e.g., monocytes/macrophages) is determined based on one or more criteria such as disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the activated immune cells being administered. For example, in some aspects, the number of monocytes or macrophages administered in the dose is determined based on the tumor burden that is present in the subject immediately prior to administration of the initiation of the dose of cells.

The pro-inflammatory agent, the SHP-1 inhibitor and/or the immune cells (e.g., monocytes/macrophages) can be administered by any suitable means, for example, by bolus infusion, by injection, e.g., intravenous or subcutaneous injections. In some embodiments, the pro-inflammatory agent, the SHP-1 inhibitor and/or the monocytes or macrophages are administered systemically (e.g., intravenously, subcutaneously, or intraperitoneally). In some embodiments, the pro-inflammatory agent, the SHP-1 inhibitor and/or the monocytes or macrophages are administered locally (e.g., intratumorally).

In some embodiments, the pro-inflammatory agent, the SHP-1 inhibitor and/or the immune cells (e.g., monocytes/macrophages) are administered by parenteral, intrapulmonary, and intranasal, and, if desired for local treatment, intralesional or intratumorally administration. Parenteral infusions include intramuscular, intravenous, intraarterial, intraperitoneal, or subcutaneous administration. In some embodiments, the pro-inflammatory agent and/or the SHP-1 inhibitor are administered orally.

In some embodiments, the immune cells (e.g., monocytes/macrophages) and the pro-inflammatory agent are administered simultaneously. In some embodiments the monocytes or macrophages and the pro-inflammatory agent are administered concurrently. In some embodiments, the immune cells (e.g., monocytes/macrophages) and the pro-inflammatory agent are administered sequentially. In some embodiments, the immune cells (e.g., monocytes/macrophages) and the pro-inflammatory agent are administered within about 7, 6, 5, 4, 3, 2, or 1 day. In some embodiments, the immune cells (e.g., monocytes/macrophages) and the pro-inflammatory agent are administered within about 24, 16, 12, 8, 4, 2, or 1 hour. In some embodiments, the immune cells (e.g., monocytes/macrophages) and the pro-inflammatory agent are administered within 30 minutes.

In some embodiments, the SHP-1 inhibitor and the pro-inflammatory agent are administered simultaneously. In some embodiments, the SHP-1 inhibitor and the pro-inflammatory agent are administered concurrently. In some embodiments, the SHP-1 inhibitor and the pro-inflammatory agent are administered sequentially. In some embodiments, the SHP-1 inhibitor and the pro-inflammatory agent are administered within about 7, 6, 5, 4, 3, 2, or 1 day. In some embodiments, the SHP-1 inhibitor and the pro-inflammatory agent are administered within about 24, 16, 12, 8, 4, 2, or 1 hour. In some embodiments, the SHP-1 inhibitor and the pro-inflammatory agent are administered within 30 minutes.

It is also contemplated that SHP-1 inhibitors and/or pro-inflammatory agents described herein can be delivered via any proper vehicles or methods. In some embodiments, the SHP-1 inhibitor and/or the pro-inflammatory agent are directly delivered into the tumor tissue. Different carrier systems can be utilized for this purpose. See e.g., Manzari et al. Targeted drug delivery strategies for precision medicines. Nat Rev Mater 6, 351-370 (2021); Tewabe et al., J Multidiscip Healthc. 2021; 14: 1711-1724. In some embodiments, the SHP-1 inhibitor and/or the pro-inflammatory agent is delivered via a nanoparticle. In some embodiments, the SHP-1 inhibitor and/or the pro-inflammatory agent is delivered via a controlled release system. In some embodiments, the SHP-1 inhibitor and/or the pro-inflammatory agent is delivered via a biomaterial implant scaffold. In some embodiments, the SHP-1 inhibitor and/or the pro-inflammatory agent is delivered via an injectable biomaterial scaffold. In some embodiments, the SHP-1 inhibitor and/or the pro-inflammatory agent is delivered via a transdermal delivery system. See e.g., Riley et al., Nat Rev Drug Discov. 2019 March; 18(3): 175-196.

In some embodiments, the SHP-1 inhibitor and/or the pro-inflammatory agent is delivered by a cell. See e.g., Millian et al., Ther Deliv. 2012 January; 3(1):25-41. In some embodiments, the cell comprises a macrophage. See e.g., Visser et al., Front Pharmacol. 2019 Jan. 25; 10:22. In some embodiments, the cell comprises a polymer encapsulated human retinal pigmented epithelial (aRPE) cell. See e.g., Nash et al., Clin Cancer Res. 2022 Aug. 22; CCR-22-1493. In some embodiments, the cells are encapsulated in a biocompatible material (e.g., biocompatible alginate capsules as discussed in Nash et al.)

In some embodiments, the SHP-1 inhibitor and/or the pro-inflammatory agent is associated with an antibody construct. In some embodiments, the SHP-1 inhibitor and/or the pro-inflammatory agent is connected with an antibody construct with via a linker (e.g., a cleavable linker). In some embodiments, the antibody construct specifically recognizes a tumor associated antigen. In some embodiments, the antibody construct comprises an antibody recognizing a tumor antigen. In some embodiments, the antibody construct is an antibody drug conjugate (ADC).

In some embodiments, the SHP-1 inhibitor and/or the pro-inflammatory agent is a delivered via a method or device that promotes delivery into a particular organ (e.g., the organ that has a tumor). See examples of these methods or devices in e.g., Alsaggar et al., J Drug Target. 2018 June-July; 26(5-6):385-397; Zhao et al., Cell. 2020 Apr. 2; 181(1):151-167, which are incorporated by reference in their entirety.

In embodiments, the SHP-1 inhibitor is delivered via a controlled drug delivery system (e.g., a slow release system or vehicle, e.g., a sustained release system or vehicle). Examples of such systems can be found in e.g., Adepu et al., Molecules. 2021 October; 26(19): 5905; Oh et al., Chem. Asian J. 2022, 17, e202200333, which are incorporated by reference in their entirety.

### VI. COMPOSITIONS COMPRISING THE SHP-1 INHIBITOR

The present application also provides compositions (e.g., pharmaceutical compositions) comprising the SHP-1 inhibitor, the pro-inflammatory agent, and/or the immune cells for treatment as described above.

In some embodiments, there is provided a composition (e.g., a pharmaceutical composition) comprising a SHP-1 inhibitor (e.g., TPI-1 or an analog or a derivative thereof) and a pro-inflammatory agent (such as any of the pro-inflammatory agents described here). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a TLR agonist (e.g., CpG, polyL:C and/or R848). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a STING activator (e.g., cGAMP, e.g., 2′3′-cGAMP, e.g., 3′3′-cGAMP). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a chemotherapeutic agent (e.g., azathioprine (AZA), e.g., gemcitabine). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a pro-inflammatory cytokine (e.g., IL-1b, IL-18, IL-6, and/or TNFα). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a checkpoint inhibitor (e.g., an anti-PD-L1 antibody, an anti-PD-1 antibody or an anti-CLTA4 antibody). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a bacteria component (e.g., LPS). In some embodiments, the composition further comprises a pharmaceutically acceptable carrier. In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent that promotes immunogenic cell death (ICD). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent used in a radiation therapy (such as any of the radiation therapy described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a PAMP/DAMP activator (such as any of the PAMP/DAMP activators described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and a cancer vaccine (such as any of the cancer vaccines described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an oncolytic virus (such as any of the oncolytic viruses described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent used in a sound treatment (such as any of the sound treatments described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent used in a magnetic therapy (such as any of the magnetic therapies described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent used in electrical or electrochemical treatment (such as any of the electrical or electrochemical treatments described herein). In some embodiments, the composition further comprises immune cells (such as monocytes or macrophages described herein). In some embodiments, the composition further comprises a pharmaceutically acceptable carrier.

In some embodiments, there is provided a composition (e.g., a pharmaceutical composition) comprising a SHP-1 inhibitor (e.g., TPI-1 or an analog or a derivative thereof) and an agent used in an electrostatic treatment (such as any of the electrostatic treatments described herein). In some embodiments, the composition further comprises immune cells (such as monocytes or macrophages described herein). In some embodiments, the composition further comprises a pharmaceutically acceptable carrier.

### EXAMPLES

The examples below are intended to be purely exemplary of the invention and should therefore not be considered to limit the invention in any way. The following examples and detailed description are offered by way of illustration and not by way of limitation.

### Example 1

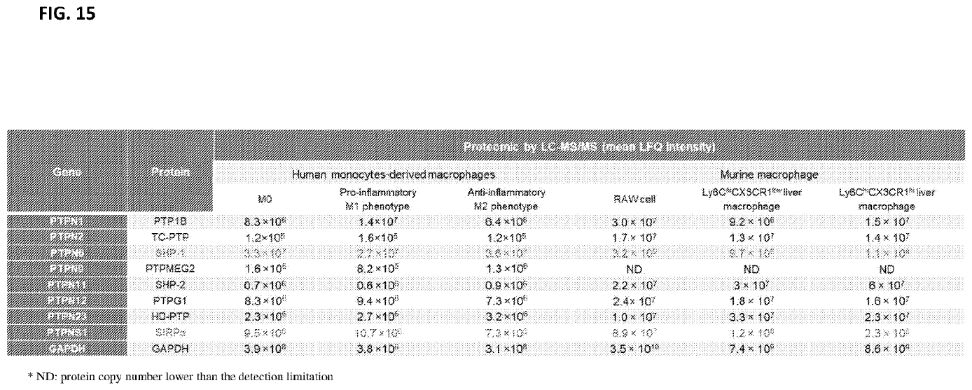

SHP-1 is abundantly expressed in macrophages. Proteomic studies examining expression of non-receptor protein tyrosine phosphatases (PTPs) in macrophages reveal that SHP-1 has the highest expression level. See FIG. 15. As shown, SHP-1 is the most abundant protein tyrosine phosphatase expressed in macrophages of both human and murine origins. SIRPα is a macrophage inhibitory receptor (iR) that involves in activation of SHP-1. Human monocytes-derived macrophages were either kept non-stimulated (M0), or stimulated with IFNγ/LPS (M1) or IL-4 (M2) to induce phenotypic activation prior to analyses. (The house-keeping gene GAPDH was detected in parallel.)

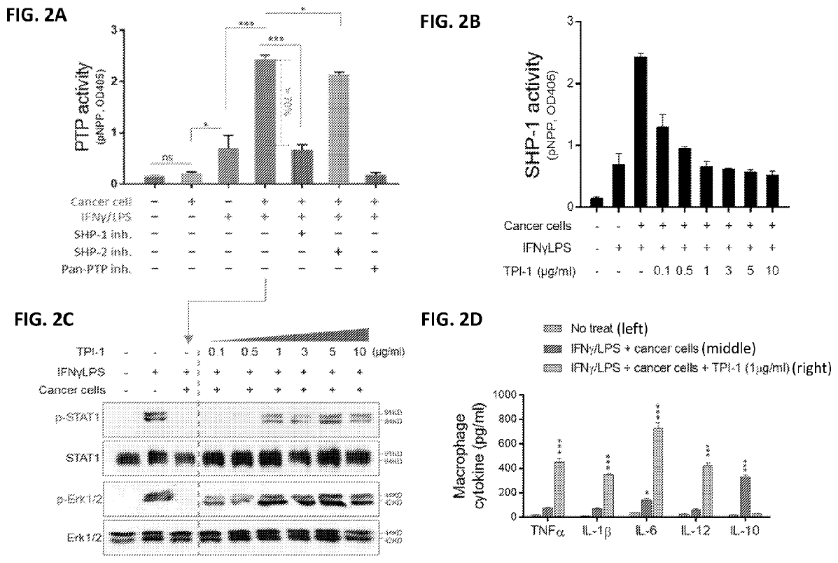

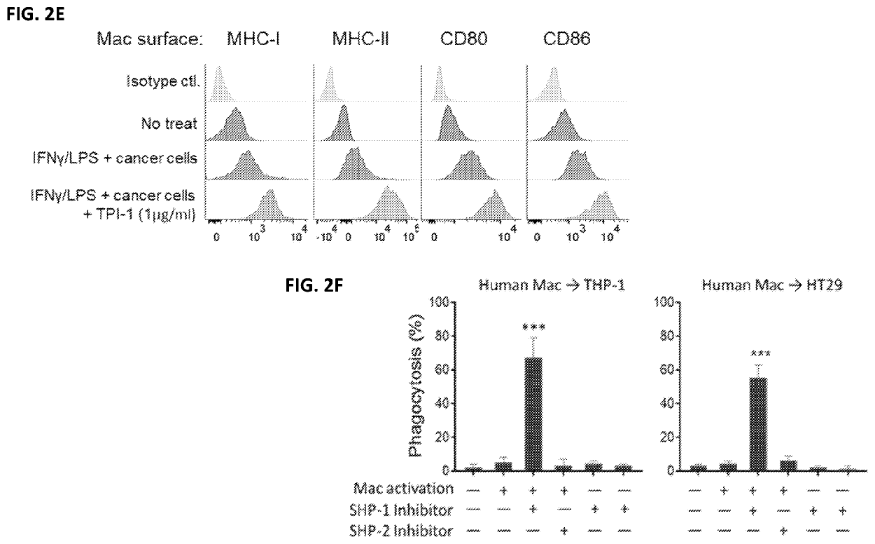

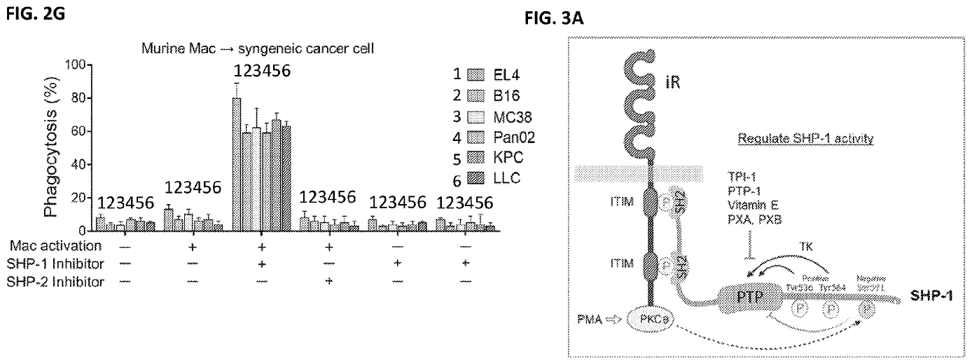

Despite its high expression, we found in tumor-pertaining macrophage assays that the SHP-1 activity remains low in macrophages unless macrophages are “surrounded/touched” by tumor cells and are simultaneously stimulated by TLR agonists or other proinflammatory activators. See FIG. 2A. Inhibition of SHP-1 activity under these conditions by TPI-1 (8), a covalent inhibitor, diminished SHP-1-mediated protein de-phosphorylation, resulting in markedly enhanced TLRs- and other factors-mediated signal transduction.

As shown (FIG. 2C), macrophages stimulated by IFNγ/LPS were completely inhibited from phosphorylation and hence activate STAT-1 (pSTAT-1) and Erk1/2 (pErk1/2) in the presence of cancer cell ligation, whereas inhibition of SHP-1 by TPI-1 dose-dependently released cancer cell-imposed inhibition allows signal transduction and activation of STAT-1 (pSTAT-1) and Erk1/2 (pErk1/2). Concordantly, this effect of SHP-1 inhibition by TPI-1 led to elevated macrophage production of proinflammatory cytokines and expression of immunogenic antigen presentation machinery (FIG. 2D and FIG. 2E). Macrophages with high SHP-1 activity in a tumor milieu diminish production of proinflammatory cytokines but produce high IL-10 (see FIG. 2D, blue bar) under stimulation of IFNγ/LPS. This reinforcement of immunosuppression was reversed by SHP-1 inhibition. Additionally, inhibition of SHP-1 also enabled proinflammatory-activated macrophages to overcome the “don't-eat-me” barrier and initiate potent phagocytosis towards cancer cells, irrespective of cancer cells expressing of CD47 (FIG. 2F and FIG. 2G).

### Example 2

In addition to TPI-1, several other SHP-1 inhibitors such as PTP inhibitor-I, PTP inhibitor-III, and recently reported vitamin E (9, 10) and phomoxanthone A and B (P×A and P×B) (11) were tested. Given that PKCθ modulates SHP-1 activity through phosphorylating Ser591 (12, 13), PKCθ inhibitor and activator were also tested in various assays.

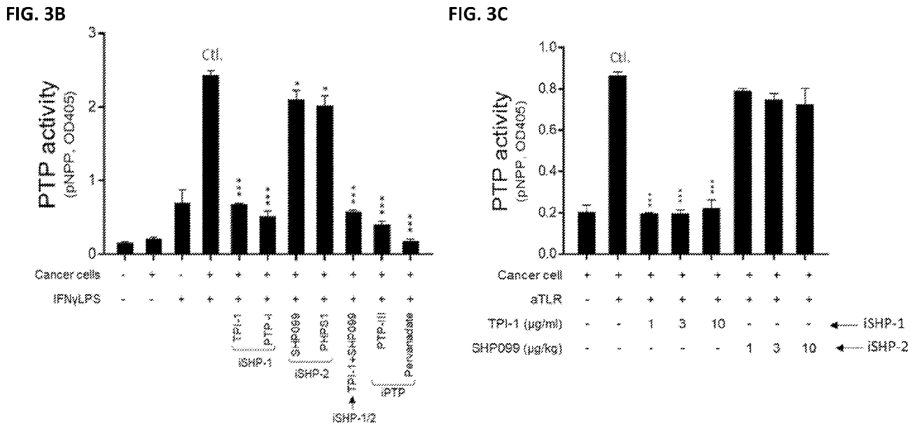

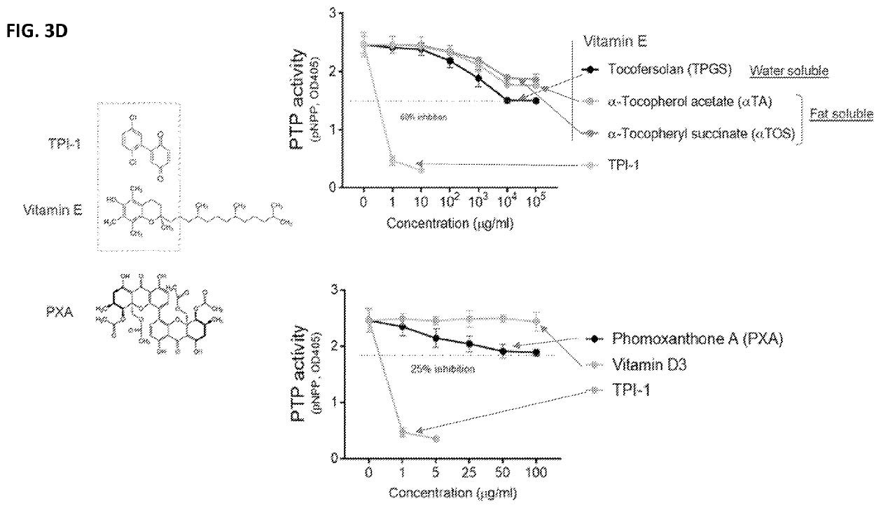

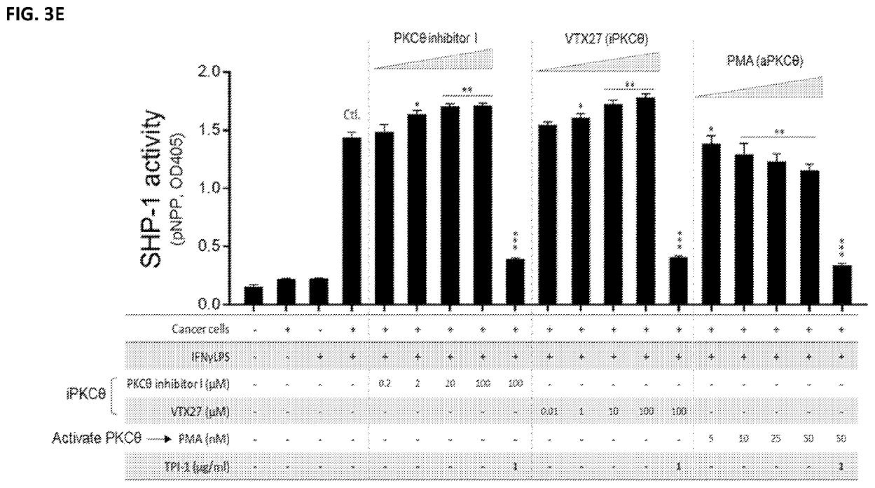

Shown in FIGS. 3A-3E, among these compounds, TPI-1 demonstrated the strongest effect and potently inhibited SHP-1 activity at low concentrations. Vitamin E derivatives and PKCθ activator PMA moderately inhibited SHP-1. We also studied effects of inhibition of SHP-2, a close family member of SHP-1. In contrast to that of SHP-1, inhibition of SHP-2 did not profoundly diminish PTP activity induced by proinflammatory activation and cancer cell ligation, nor did it bestow activated macrophages for phagocytosis of cancer cells. These studies concluded that SHP-2 is regulated differently from that of SHP-1, and that SHP-1, but not SHP-2, controls macrophage proinflammatory response, immunogenic antigen presentation and phagocytosis towards cancer cells.

### Example 3

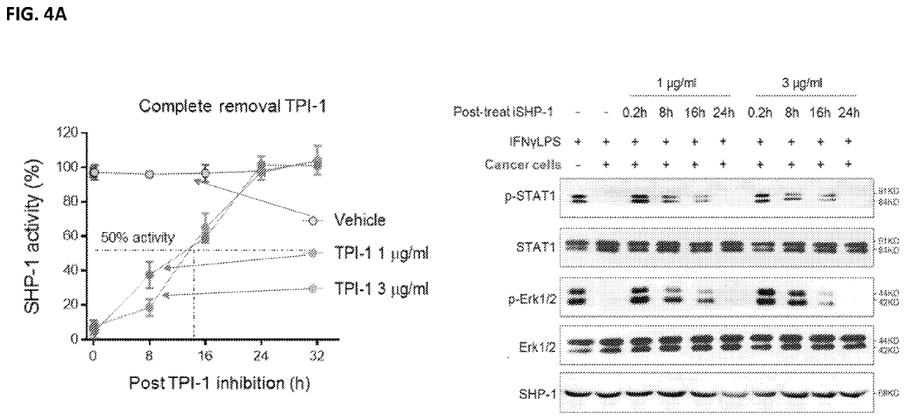

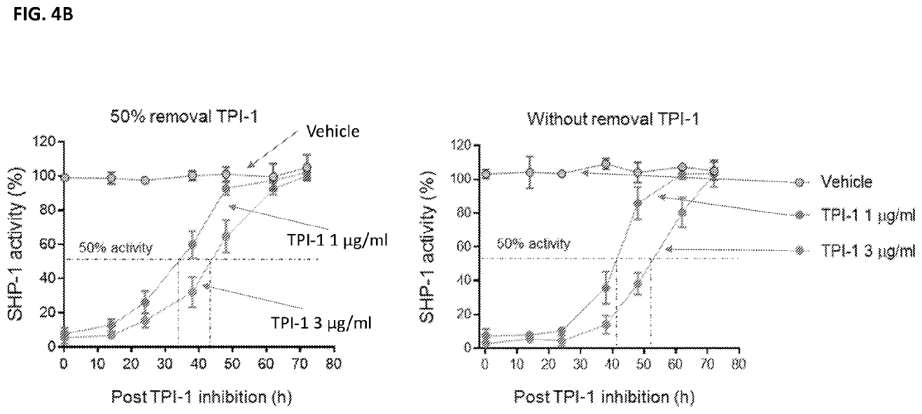

It was found that SHP-1 has a rapid turnover rate. Treating macrophages with the covalent inhibitor TPI-1 for 15 min, followed by wash and removal of the inhibitor availability, observed strong SHP-1 inhibition immediately after the TPI-1 treatment, an effect associated with increased pSTAT-1 and pErk1/2 by IFNγ/LPS. However, this manner of pulse SHP-1 inhibition did not last beyond a few hours (5-8 h) when the activity of SHP-1 started to recover and reached over 50% in 12-16 h, gaining ability to diminish IFNγ/LPS-induced signal transduction (FIG. 4A). Despite SHP-1 activity changes, its total protein did not appear to vary. Treating macrophages with TPI-1 without the inhibitor removal, or with partial removal, elongated inhibition on the SHP-1 activity (FIG. 4B).

### Example 4

Further molecular and cell signaling studies demonstrated that, in a tumor milieu, tumor cells through their cell surface counter-receptors (e.g. CD47, MHC and carbohydrate) ligate macrophage iRs (e.g. SIRPα, LIlRBs and Siglecs), which then drive high-level SHP-1 activation through their cytoplasmic ITIMs that undergo tyrosine phosphorylation in the presence of proinflammatory stimuli and bind to and, as such, dislodge the SH2 domain from autoinhibition of SHP-1 (depicted in FIG. 5A). Neither iR extracellular ligation nor proinflammatory stimulation alone is capable of driving strong ITIMs phosphorylation and hence activation of SHP-1, but their simultaneous presence achieves this. The underlying mechanism suggests that the extracellular ligation of iRs induces their cytoplasmic structural changes to an “open” form, exposing ITIMs to phosphorylation, while proinflammatory signals simultaneously induce activation of Src family tyrosine kinases (TK) to mediate phosphorylation of ITIMs. FIG. 5B shows the study of SIRPα, an essential macrophage iR whose ITIMs phosphorylation requires both extracellular ligation of CD47 and macrophage stimulation by cytokines or TLR agonists.

These studies also found that macrophage iRs, such as SIRPα, are capable of recruiting both SHP-1 and SHP-2 through the cytoplasmic ITIMs phosphorylation. However, SHP-1 binding occurs exclusively under proinflammatory conditions when macrophages are stimulated by activating cytokines (TNFα, IL-17A, IL-6, or IFNγ) or TLR agonists (LPS, CpG, or PolyIC), whereas SHP-2 binding is dominated by immunosuppressive IL-4, IL-10 or TGFβ (FIG. 5C).

### Example 5

Similar findings were made in vivo in solid tumors. With tumors progressing to large sizes, intratumoral macrophages were found to increase expression of iRs, while tumor cells in the same TME also elevated their counter-receptors, such as CD47, and T cell inhibitory molecule PD-L1 (FIG. 6A). These changes in late-stage tumors suggest that compared to their early stage, they have established stronger immunosuppression. In the absence of therapy, intratumoral macrophages constantly ligated by surrounding tumor cells are directed by the TME immunosuppressive signals, resulting in their iR phosphorylation in cytoplasmic ITIMs and binding of SHP-2, not SHP-1 (data unshown). Our accumulative data indicate that this manner of iRs-SHP-2 binding sequestrates SHP-2 from access to immunosuppressive cytokine receptors (e.g., IL-4R and IL-10R) and, as such, prevents SHP-2 from inhibiting anti-inflammatory signal transduction (14). Thus, this iRs-SHP-2 binding in homeostatic solid tumor serves as a feed-forward regulation that reinforces the anti-inflammatory phenotype of macrophages, promoting the TME immunosuppression and tumor progression.

The SHP-1 activity in non-therapeutic solid tumors is low (see FIGS. 7A-7D), and this low activity inhibits immunosuppressive receptor signaling. Indeed, this function of SHP-1 synergizes SHP-2, both capable of binding to IL-4R and IL-10R and deactivating their signal transduction (see our previous study, and studies by others (14-17). In consistency, inhibition of SHP-1 in MC38 solid tumor resulted in increased IL-10 production in the TME (FIG. 6B). Interestingly, inhibition of SHP-1 also increased tumor production of IL-6, a cytokine that is reported to play an immunosuppressive role in TME and support tumor progression (18, 19). In vitro assays of macrophage activation in a tumor-pertaining setting with alternative activation (M2) stimuli IL-4, IL-13 and IL-10 confirmed that inhibition of SHP-1 with TPI-1, or another inhibitor PTP-1, dose-dependently increased macrophage production of IL-10 and TGFβ (FIG. 6C). In parallel experiments, the same SHP-1 inhibitors augmented macrophage responses to the M1 stimuli IFNγ and LPS and enhanced proinflammatory cytokine production.

Supporting this notion, we found that once tumors are therapeutically treated, such as those treated by TLR ligands (αTLR), inflammatory cytokines (IL-1/6/TNFα/IFNγ), STING activator (2′3′-cGAMP), RT, anti-PD-L1 immune checkpoint blockade (αPD-L1), or a chemotherapy drug Azacitidine (AZA), spikes of SHP-1 activity are induced (FIG. 7A and FIG. 7B). Indeed, late-stage large tumors having high expression of iRs on macrophages lend a capacity for robust activation of SHP-1 under therapeutic treatments. We found that intratumoral macrophages are the primary source of SHP-1 activity, and depletion of macrophages in tumors largely diminished the SHP-1 activity spikes upon therapies (FIG. 7C). Besides deactivating proinflammatory signaling pathways, these high SHP-1 activities also through currently unelucidated mechanisms augment proinflammatory stimuli-induced IL-10 and TGFβ (FIG. 7D, also see FIG. 2D), thus accelerating the TME to resume immunosuppression. Consequently, solid tumors with high inductions of SHP-1 activity under treatments with TLR agonists or other therapies failed to induce intratumoral macrophage phenotypic change from immunosuppression to proinflammation, nor did they reprogram the TME towards an anti-tumor immunogenicity, such as what we observed in pancreatic ductal adenocarcinoma (KPC) and colorectal carcinoma (MC38) under treatments (FIGS. 7D-7F). Rather, these tumors arbitrated strong resistance to therapies through increasing TGFβ and TGFβ receptors (TGFBR1 and TGFBR2) and CCL2 chemokines that attract MDSC (FIG. 7F), leading to wound healing and reinforcement of immunosuppression. With strong treatment resistance, there was no or only marginal-level antigen presentation detected in the TME and anti-tumor T cell immunity was curbed low.

In stark contrast, inhibition of SHP-1 with a single dose of TPI-1 completely altered how the TME responded to TLR agonists or RT. Merely a few hours (6-18 h) post treatment (TPI-1+αTLR or RT), intratumoral macrophages altered their phenotype from immunosuppression to characteristic proinflammation, featuring high expression of proinflammatory cytokines TNFα, IFNα/p, IFNγ, IL-1β, IL-6, IL-12, IL-17, IL-18, etc., while also abating IL-10 and TGFβ. A prominent immunogenic antigen presentation machinery was induced, with elevated cell surface markers MHC-I, MHC-II, and costimulatory molecules CD80, CD86, CD40, OX40L, etc. A panel of chemokines that attract neutrophils, NK and T cells were also increased in the TME, whereas TGFRs and CCL2 were reduced (FIGS. 7D-7F).

### Example 6

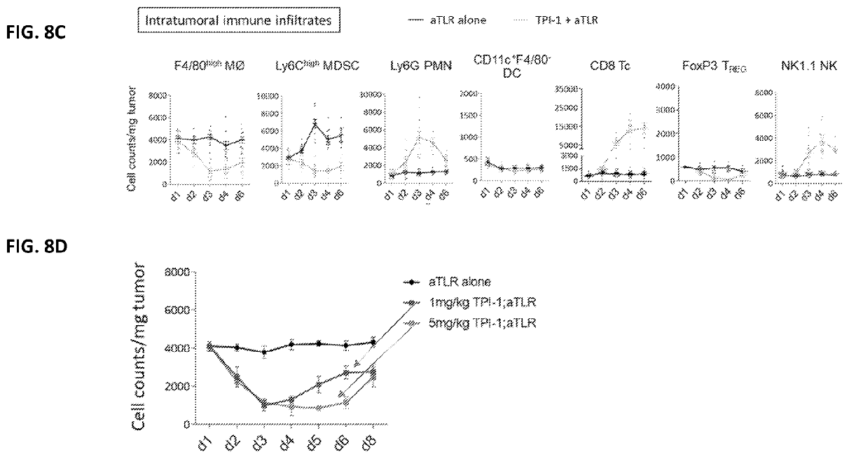

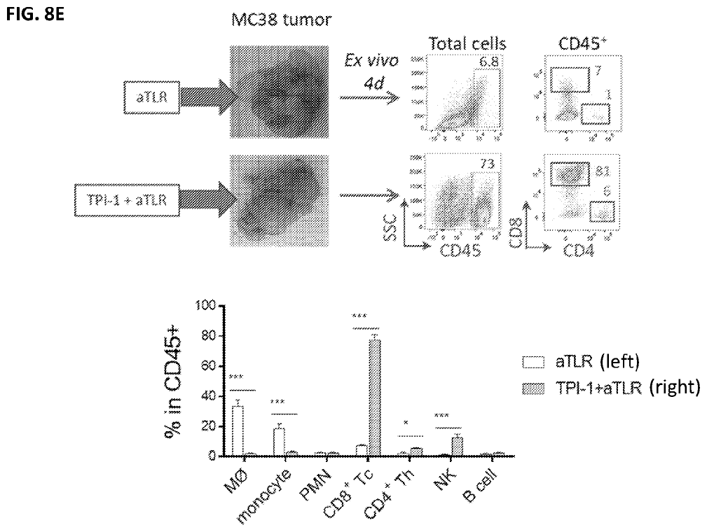

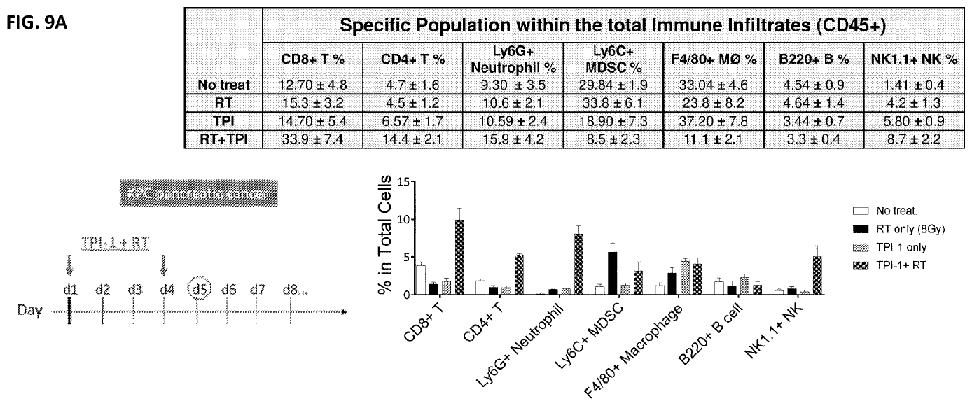

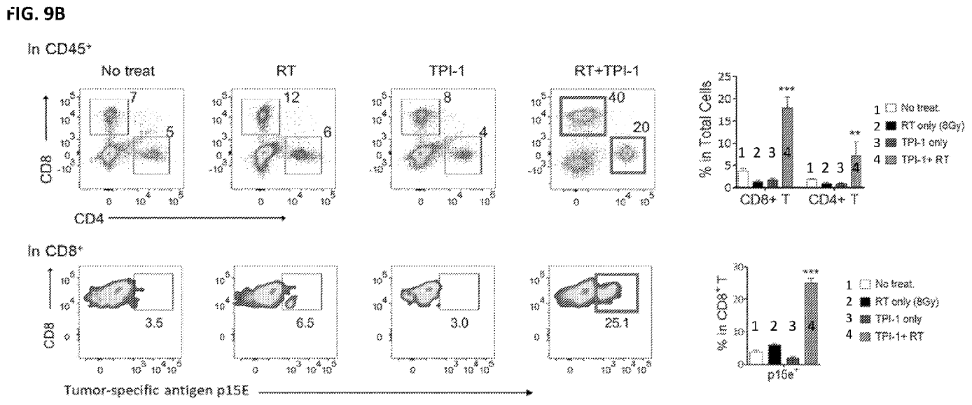

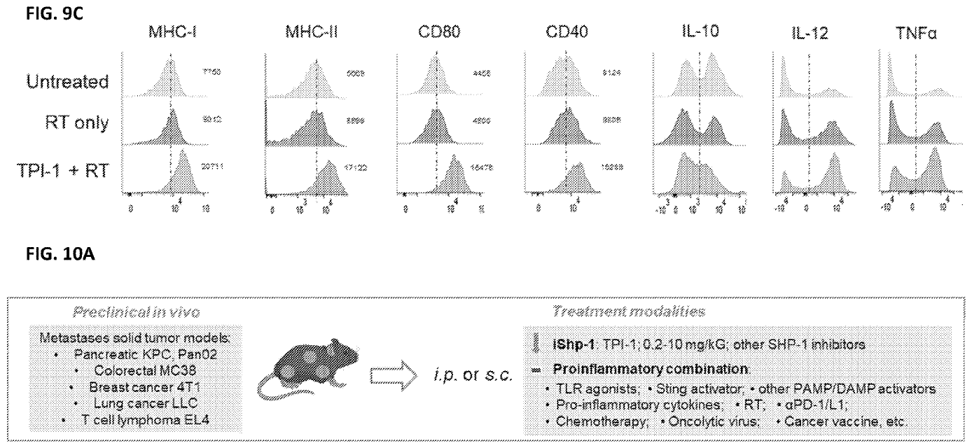

Consistent with these changes, TME analyses of MC38 colorectal and KPC pancreatic tumors treated with TLR agonist combined with TPI-1 or RT (FIGS. 8A-8E & 9) found rapid infiltration of inflammatory neutrophils (ROShigh) and tumor-killing NK cells (Granzhigh), and the strong antigen presentation also brought about the expansion of tumor-specific (p15E-reactive) cytotoxic T cells harboring high tumoricidal capacity (Granzhigh). The immunosuppressive compartments in the same TME, including MDSC and Treg, were reduced. Interestingly, the intratumoral population of macrophages (F4/80+) was also reduced to a minute size following T cell activation. We further found that this event of T cell activation and expansion was driven chiefly through intratumoral APC-mediated antigen presentation in situ that activated tumor-specific memory T cells (TEM/CM) in TIL. Ex vivo infusion of TLR agonists and TPI-1 together, but not each individually, into excised tumors induced the similar response of T cell expansion. However, treating excised tumors did not bring about increases in neutrophils and NK cells, suggesting that increased infiltration of these killing cells in tumors in vivo were through chemotactic recruitment extratumorally. Together, these results suggest that inhibiting SHP-1 to remove the central mechanism hurdling proinflammatory signal transduction unbridles intratumoral anti-tumor potentials under therapy, empowering both innate and adaptive immune cells against cancer.

Furthermore, these analyses of the TME immune landscape changes following TPI-1 combined therapies informed our design of the pulse-intermittent SHP-1 inhibition strategy for metastatic solid tumors. We found in tumors treated with TPI-1 combined with TLR agonists or RT that the population of intratumoral macrophages in the TME greatly reduced following antigen presentation to activate T cells. Along with macrophage reduction, tumor-associated SHP-1 activities also dwindled, a change that ousted the necessity of continuous TPI-1 application. This “intermittent” period lasted 3-6 days, a length dependent on TPI-1 and TLR agonists/RT dosages, as well as tumor type and stage prior to treatment. During this period, the TME was dominated by tumor-killing CD8 T cells, neutrophils, and NK cells, and the treated tumors were restrained from growth but displayed a state of stable disease (SD) or tumor regression. However, providing the tumor is not completely eliminated, the TME was later re-populated with ‘new’ macrophages and showed increased MDSC and Treg, suggesting re-establishment of immunosuppression. Consistent with these changes, the tumors after the intermittent period were reverted to growth, unless another cycle of TPI-1 combined therapy was applied, which were again effectively suppressing tumors.

### Example 7

Our design of pulse-intermittent inhibition of SHP-1 (iSHP-1) as an immune adjuvant/neoadjuvant therapy to solid tumor arises from the above mechanistic studies. Based on our findings, iSHP-1 monotherapy presents little rationale, but iSHP-1 combined with proinflammatory regimens predicts potent tumor suppression. Given that intratumoral macrophages dictate the TME response and SHP-1 activity in solid tumor, the iSHP-1 strategy is tailored to target intratumoral macrophages and exploits their capacity to drive proinflammatory response and antigen presentation for tumor elimination. Following the intratumoral macrophage population dynamics, the iSHP-1 treatment is given in a “pulsed” fashion when macrophages are abundant within the TME, but withheld when they diminish following antigen presentation to activate T cells (“intermittent” period). In addition, given our finding that macrophages re-populate the TME after the intermittent period, providing the tumor is not eliminated, multiple cycles of treatment are designed as each cycle repeats the pulsed iSHP-1 right after the intermittent period.

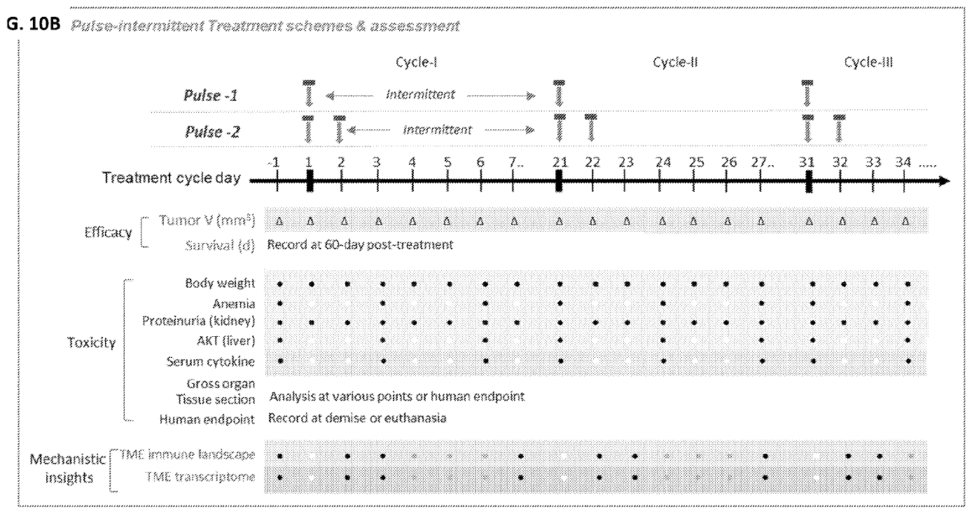

FIGS. 10A-10B depicts pulse-intermittent iSHP-1 design and treatment schemes. In preclinical tumor models, we have tested three pulsed treatment schemes, pulses-1, -2 and -3, with iSHP-1 given once, twice or three times consecutively (1× per day) at the beginning of each cycle, and an intermittent period of 2-9 days withholding iSHP-1 treatment between cycles. The combination modalities include, but are not limited to, TLR ligands, STING activator, RT, anti-PD-1/L1 immune checkpoint blockade (αPD-1/L1), inflammatory cytokines, chemotherapies and oncolytic viruses. These combination regimens were given together with SHP-1 inhibitor or following separate dosing schedules. Murine solid tumor models with a single tumor or multi-lesions (metastases) in syngeneic mice of different backgrounds were tested. These models were established by engrafting tumors in multiple locations, and iSHP-1 treatment began when a single tumor reaches ≥200 mm³ in size or the total tumor burden ≥300 mm³. These models included pancreatic adenocarcinoma (KPC and Pan02), colorectal carcinoma (MC38), metastatic breast cancer 4T1, lung cancer (LLC), and T cell lymphoma (EL4). All treatments to mice were given in a systemic fashion, via intraperitoneal (i.p.) or subcutaneous (s.c.) administration, in order to achieve a whole-body effect on ‘metastatic’ lesions. Treatments were also given via intratumoral injection (i.t.) in a subset of experiments.

Throughout treatments, anti-tumoral efficacies (tumor volume changes and survival rates), adverse effects (body weight loss, proteinuria, anemia and clinical discomfort), gross organ toxicity and tissue inflammation due to toxicity, and the TME immune landscape changes were analyzed at different time points and at the terminal point.

As seen in the next few examples, we have intensively vetted pulse-intermittent iSHP-1 strategy in preclinical tumor models, and in all cases, pulsed iSHP-1 combined with proinflammatory regimens induced potent anti-tumoral responses with activation of innate and adaptive immune cells together to eliminate tumor. Application of this strategy in a systemic approach led to control and regression of cancer lesions in the whole body, resulting in high rates of survival and a long-lasting anti-tumor immunity.

More importantly, the pulse-intermittent iSHP-1 strategy, for the first time, provides a practical method that allows SHP-1 inhibition to be possibly applied as a therapeutic regimen in vivo, for this strategy maximally minimizes the SHP-1 deficiency-incurred toxicity. Compared to animals deficient of SHP-1 or treated with SHP-1 inhibitor continuously that bashed into overt lung inflammation, kidney damage, colitis, anemia and splenomegaly, mice with three cycles of pulse-intermittent iSHP-1, with TPI-1 doses adequate to induce systemic tumor elimination, combined with TLR ligands, STING activator, RT, αPD-1/L1 and proinflammatory cytokines, did not exhibit serious lung or kidney damage, anemia or splenomegaly (data shown in the next section, case studies). This high beneficial vs risk effect attributes to the pulse intermittent iShp-1 design, which is specially tailored for targeting intratumoral macrophages to achieve tumor-focal efficacies, and which precludes lengthened depletion of SHP-1 activity in essential organs, e.g., lung, intestines, kidney and spleen, where SHP-1 activity is critical for preventing unwanted autoimmune inflammatory response against commensal microbials or debris particles. Our further studies supported this notion, demonstrating that following a pulse inhibition of SHP-1 in macrophages with the covalent inhibitor TPI-1, macrophages were able to recover the SHP-1 activity in 24 h.

### Example 8

We have tested iSHP-1 combined with TLR agonists (αTLR), STING activator, immune checkpoint blockade (αPD-1/L1), and/or RT to treat various solid tumors. These include pancreatic ductal adenocarcinoma KPC (KPC-luc) and Pan02, colorectal carcinoma MC38, lung cancer LLC, and metastatic breast cancer 4T1 in murine syngeneic models with complete immune competency. Both treatment efficacies and therapeutic safety were assessed according to FIG. 10.

Murine models (single or multi-lesion solid tumors): 1) Pancreatic ductal adenocarcinoma (KPC or KPC-luc)—C57BL6 syngeneic engraftment, 2) Colorectal carcinoma (MC38)—C57BL6 syngeneic engraftment, 3) Lung cancer (LLC or LLC-luc)—C57BL6 syngeneic engraftment, and 4) metastatic breast cancer (4T1, or 4T1-luc)—BalbC background syngeneic engraftment.

Establishing tumor models: Healthily cultured cancer cells (1-5×10⁵) suspended in 50 μL PBS were subcutaneously (s.c.), intraperitoneally (i.p.), intravenously (i.v.), or orthotopically injected into WT C57BL6 or BalbC mice (6-8 wk, male and female) to establish syngeneic models. For multiple tumor lesions (metastases model), tumor cells were engrafted at multiple locations through multi-point s.c. injections combined with i.p. or i.v. Palpable subcutaneous or orthotopic (e.g., 4T1) tumors formed after 10-14 days. Measurements using calipers to record the tumor length and width, followed by calculation of the tumor volume (V) with formula: volume=(length×width2)/2. For tumor cells expressing luciferase (KPC-luc, LLC-luc, 4T1-luc), whole-body images for luminescence intensity were taken to display tumors.

Therapeutic treatment: Treatments started when a single tumor grew to ≥200 mm³, or the total tumor burden ≥300 mm³. The covalent SHP-1 inhibitor TPI-1 was chosen given its strong inhibitory capability and relative specificity. TPI-1 of varied doses in PBS was given following different dosing strategies via i.p. or s.c. injection for a systemic effect at locations distant from the tumor sites. In a subset of experiments, the effect of TPI-1 was also tested by direct injection into tumors (intratumoral injection, i.t.). TPI-1 was given prior to or together with combination modalities.

A. Study-1 (FIGS. 11A-11E): Continuous Vs Intermittent iSHP-1 Combined with TLR Agonists for Therapeutic Efficacy and Adverse Toxicity

Tumor model: Single engraftment of KPC pancreatic adenocarcinoma

Treatments & dosing strategies: i) iSHP-1—TPI-1, 1, 3, and 10 mg/kg; i.p. either 1× per day (continuous), or following an intermittent schedule depicted in FIG. 11. ii) TLR agonists (αTLR)—CpG, PolyI:C, each 10 μg; i.p. every 3 days

Results: TPI-1 exhibited dose-dependent effects and its combination with TLR agonists restrained KPC pancreatic tumor growth and induced tumor regression. Both continuous and intermittent strategies of TPI-1 administration achieved similar anti-tumoral efficacies. However, continuous dosing of TPI-1 (1× per day) incurred acute anemia, proteinuria, splenomegaly and lung inflammation. These adverse effects did not display or were minor in mice intermittently treated with TPI-1. In conclusion, the intermittent iSHP-1 strategy greatly reduced risks of adverse toxicity while achieving tumor suppressing efficacies.

B. Study-2 (FIGS. 12A-12D): Pulse-Intermittent iSHP-1 Combined with TLR Agonists and/or ICB (αPD-L1) Treating Multi-Lesion Colorectal Carcinoma, a Study for Efficacy and Adverse Toxicity

Conclusion: i) Intermittent iSHP-1 combined with TLR agonists (αTLR), or αTLR and αPD-L1, effectively regressed multiple lesions of MC38 colorectal cancer, which otherwise resisted to TLR alone or αPD-L1. ii) TPI-1 administrated via i.p. and s.c. had similar systemic efficacies. iii) αPD-L1 enhances iSHP-1 plus αTLR mediated activation of T cell immunity. iv) Intermittent iSHP-1 therapies did not incur acute adverse toxicity.

C. Study-3 (FIGS. 13A-13E): Pulse-Intermittent iSHP-1 Combined RT and αPD-L1 Treating Multi-Lesion Pancreatic Ductal Adenocarcinoma and Lung Cancer

Modalities & Dosing Strategy: i) iSHP-1—TPI-1, 3 or 5 mg/kg, i.p., 1× every 3 days. ii) Tumor focal RT: 8Gy-4Gy-2Gy, given to right flank simultaneously with iSHP-1. iii) αPD-L1: 100 μg, i.p., given a day after iSHP-1 plus RT

Results: Systemic intermittent iSHP-1 combined with the right flank tumor-focal RT, followed by αPD-L1 to bolster T cell immunity, induced strong anti-tumor immunity with abscopal effects, effectively eliminating or repressing KPC pancreatic cancer and LLC lung cancer along with distal lesions.

D. Study-4 (FIGS. 14A-14E): Pulse-Intermittent iSHP-1 Combined with TLR Agonists Treating Late-Stage, Large KPC Pancreatic Cancers.

Murine models: Single large pancreatic ductal adenocarcinoma (KPC-luc) C57BL6 syngeneic

Treatments: SHP-1 inhibition (iSHP-1) combined with TLR agonists (αTLR)

Results: as shown in the FIGS. 14A-14E, TPI-1 combined with αTLR bolstered anti-tumor immune cells such as CD8+ T cells, NK cells and neutrophils and effectively eradiated late-stage, large KPC pancreatic cancers.

### Example 9

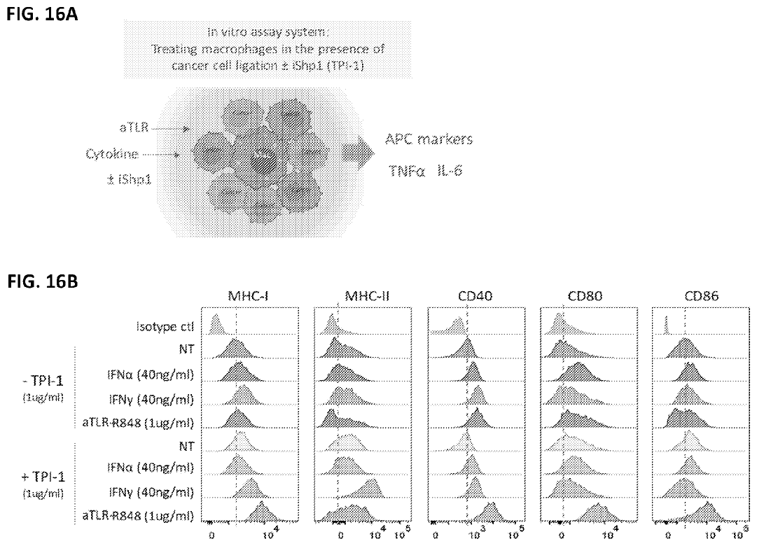

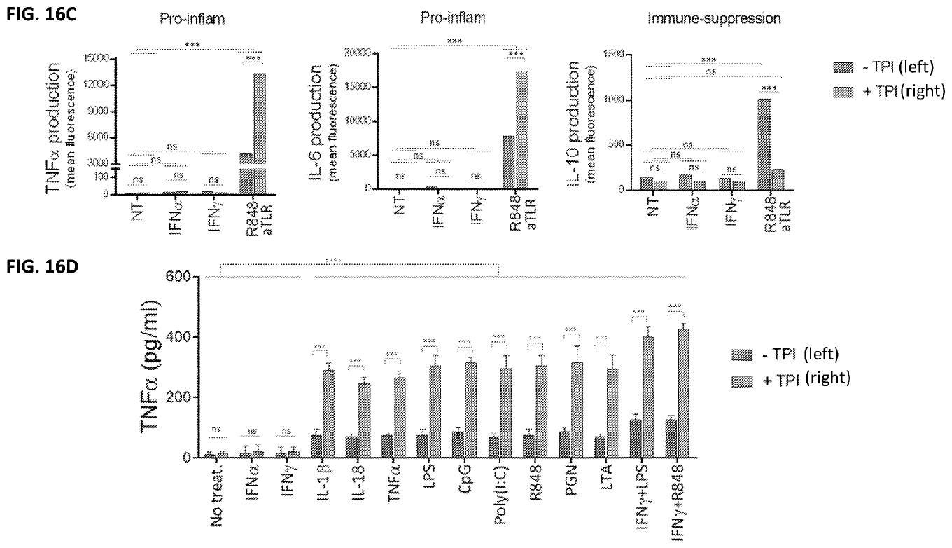

In vitro macrophages were assayed in the presence of tumor cells that ligate macrophage iRs with or without inhibition of SHP-1 (iShp1) by TPI-1. See the testing system in FIG. 16A. As shown in FIGS. 16B and 16C, IFNα treatment alone did not induce antigen presentation or proinflammatory response by macrophages without and with iShp1. IFNγ plus iShp1 significantly increased antigen presentation albeit the combination did not elicit proinflammatory cytokine production. In contrast, IL-1 family cytokines (IL-1β, IL-18), TNFα and TLR ligands combined with iShp1 exhibited capacities of pro-inflammatory activation of macrophages in a tumor milieu.

### Example 10

MC38 colorectal carcinoma was established (via subcutaneous administration) in syngeneic C57BL6 mice. After tumor sizes reached more than 200 mm³, the tumor-bearing mice were induced systemic inflammatory condition by TLR agonists (αTLR, CpG/PolyIC/R848, each 25 μg) administrated via subcutaneous administration at the location distal from MC38 tumor. A group of mice were concurrently administrated with TPI-1 (1 mg/kg) via subcutaneous administration to achieve systemic SHP-1 inhibition.

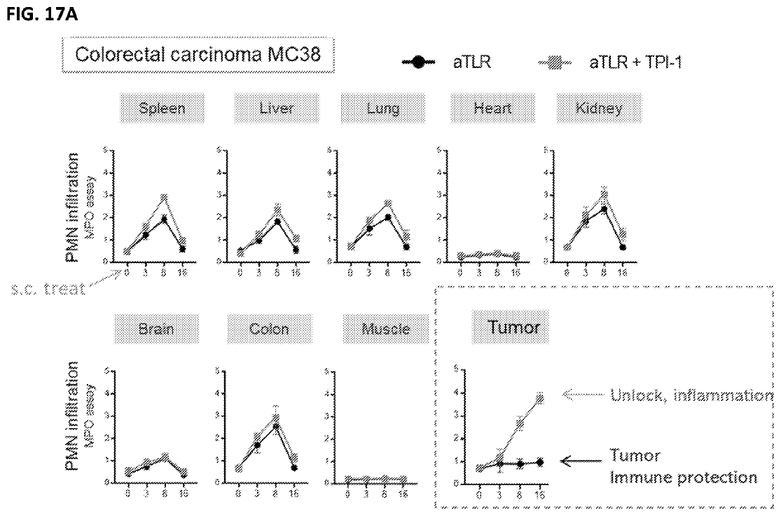

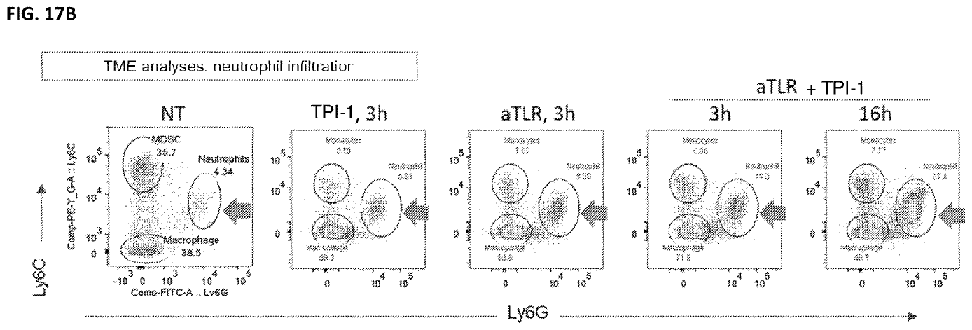

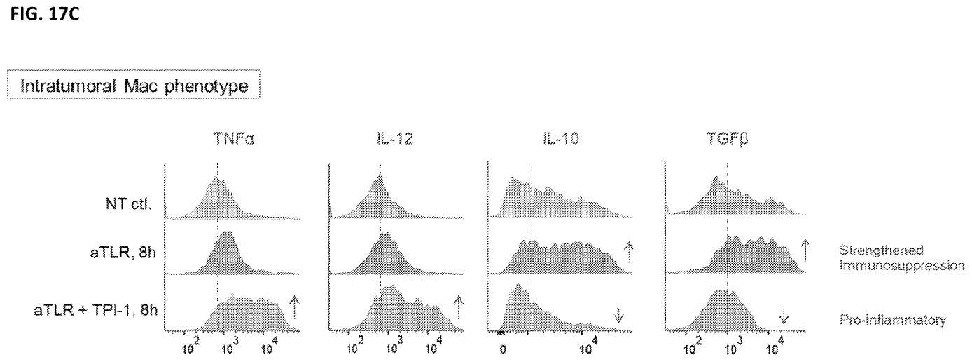

As shown in FIG. 17A, MC38 tumors were protected from αTLR-induced acute inflammation via SHP-1. Neutrophil infiltration in different organs, indicative of local tissue inflammation, were measured at various time point following αTLR challenge. Inhibition of SHP-1 by TPI-1 unlocked the tumor immunosuppression and enabled neutrophil infiltration upon αTLR (αTLR+TPI-1).

As shown in FIG. 17B, TEM analyses confirmed increased neutrophil infiltration in tumor tissues in mice treated with αTLR plus TPI-1.

As shown in FIG. 17C, inhibition of SHP-1 by TPI-1 enabled intratumoral macrophages to be skewed towards pro-inflammatory activation by αTLR, exhibiting increased TNFα and IL12 expression. In contrast, the absence of TPI-1 caused macrophages to resist proinflammation by αTLR while enhancing immunosuppression and increasing IL-10 and TGFβ expression.

### Example 11

This example demonstrates that neutralization of TNFα curbs down systemic inflammation without affecting anti-tumor efficacies by TKi, SHP-1 inhibition and αTLR combination.

Mice with established MC38 colorectal carcinoma (200-400 mm3) were treated with αTLR, TPI-1 and Dasatinib (s.c.), without or with additional treatment with anti-TNFα mAb or anti-IL-6 mAb (150 μg, i.p.). The treatment was repeated once (d1 and d2). Tumor volume changes were recorded, and tumor TMEs were analyzed for immune infiltrates on day 6 post treatments. See FIG. 11A.

As shown in FIG. 11B, tumor volume decreased following treatment of αTLR+TPI-1+Dasatinib, and the administration of anti-TNFα or anti-IL-6 did not interfere with their anti-tumor activities. Anti-TNFα mAb or anti-IL-6 mAb treatment also did not affect αTLR/TPI-1/Dasatinib therapy-induced increases in CD8 T cells (Tc) and NK cells, as well as reduction of macrophages and MDSC in the TME. See FIGS. 11C and 11D. Treating mice with anti-TNFα mAb, but not anti-IL-6 mAb, largely diminished the induction of inflammatory cytokines (TNFα, IL-6, IL-1β, IL-10, IFNα and IFNγ) associated with the αTLR/TPI-1/Dasatinib combination therapy. Anti-TNFα treatment also markedly reduced monocyte and PMN chemokines CCL2, CCL5 and CXCL1 in circulation, while without reducing CXCL10 that is essential for T cell trafficking. FIG. 11E. Further, as shown in FIG. 11F, anti-TNFα treatment protected mice from developing splenomegaly and intestinal inflammation that were commonly associated with αTLR/TPI-1/Dasatinib therapy.

Together, the results showed that neither anti-TNFα nor anti-IL-6 interfered TKi/iShp1/αTLR for driving anti-tumor immunity or achieving therapeutic efficacies. Moreover, anti-TNFα exhibited beneficial effects by largely abrogating TKi/iShp1/αTLR-induced cytokine storm and systemic inflammation, thereby curbing down the therapy-associated adverse toxicity.

Moreover, although the above specific experiment involves using both TPI-1 and dasatinib and both polyL:C and R848, our results from experiments using either one of TPI-1 and dasatinib and either one of polyL:C and R848 achieved similar effects (data not shown). We also found that the proper time window for anti-TNFα antibody treatment can be from at least a week prior to (as long as the antibody is stable for the time window) to immediately after (e.g., within 0.5-1 hour) the SHP-1 inhibitor/αTLR treatment. It is preferable that the anti-TNFα antibody is provided prior to or simultaneously with the SHP-1 inhibitor and/or αTLR so that it maximally blocks the TNFα induced after the treatment of SHP-1 inhibitor and the pro-inflammatory agent.

### Example 12

Mechanism by which tumor cells inhibit macrophage proinflammatory response in TME was studied. As shown in panel A of FIG. 19, macrophage responses were assayed in a tumor milieu: Human monocyte-derived macrophages (MØ) were cultured alone (configuration 1), cultured in the presence of cancer cells-conditioned medium (50%) that contained cancer cell secreted factors (secretome; configuration 2), or co-cultured with cancer cells (cell “touch” model; configuration 3), or co-cultured with cancer cells that were placed in a transwell (0.4 μm) without “touching” MØ(configuration 4). Macrophages in these settings were treated with proinflammatory stimuli TLR agonist R848 (l g/ml), R848 plus IFNγ (40 ng/ml), or the sting activator MSA-2 (10 μg/ml).

Macrophage responses to R848/IFNγ were then studied. The presence of cancer secretome (configurations 2) and 4)) did not change the pattern of macrophage proinflammatory response, albeit resulting in partial inhibition (10%-40% reduction) of proinflammatory cytokine production. In contrast, macrophages co-cultured with cancer cells were abolished proinflammatory response and exhibited enhanced immunosuppression with high IL-10 production. See panel B of FIG. 19.

Macrophage response to R848 or MSA-2 were also studied. Similar results as in panel B: the presence of cancer secretome partially inhibited macrophage proinflammatory response, but macrophages co-cultured with cancer cells displayed abolishment of proinflammatory response and enhanced immunosuppression with high IL-10 and TGFβ production. See panel C of FIG. 19.

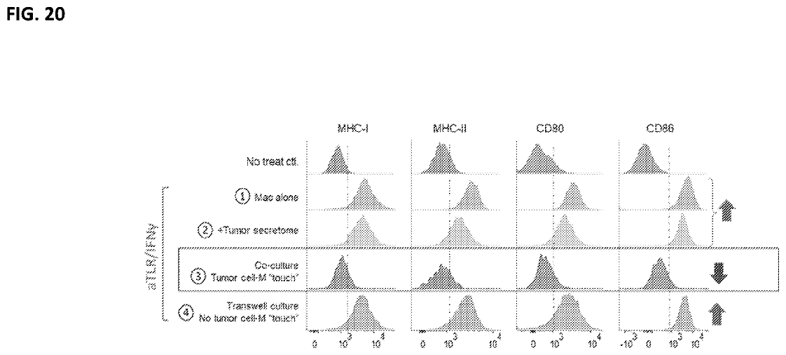

Furthermore, as shown in FIG. 20, macrophages co-cultured with cancer cells abolished TLR agonist (R848) plus IFNγ-induced macrophage antigen presentation.

### Example 13

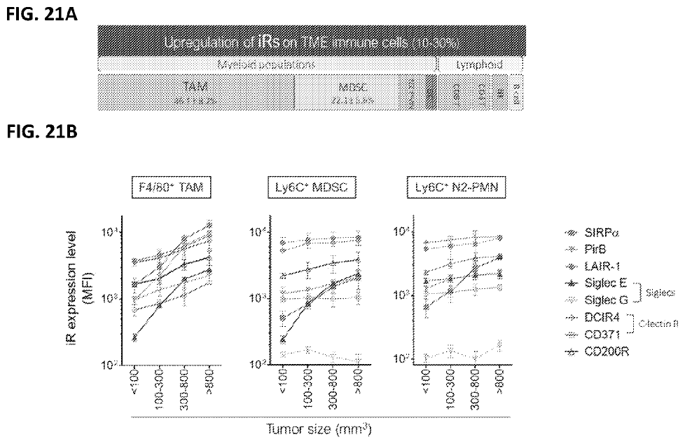

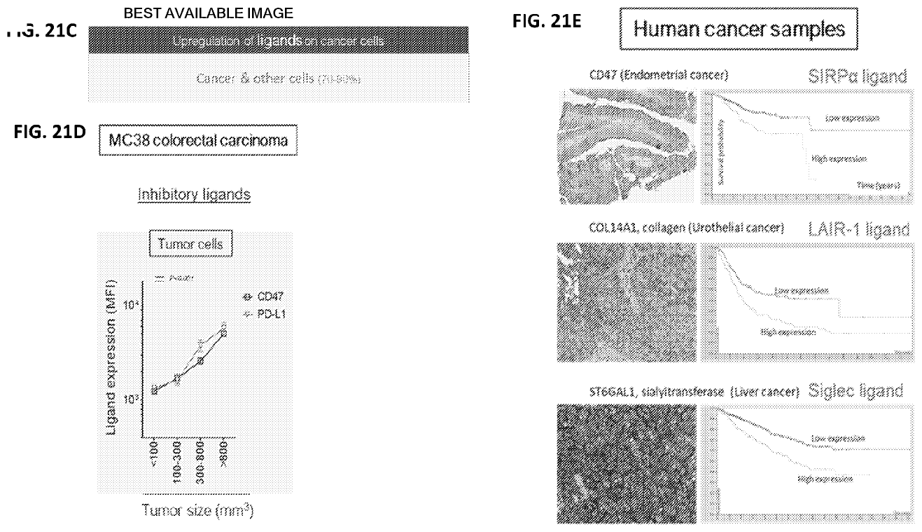

It was found that iRs and their ligands were upregulated when tumors progress to late stages (large sizes), as shown in FIG. 21. Panel A depicts immune cell compositions and percentages within a typical MC38 colorectal carcinoma. Panel B depicts expression of multiple inhibitory receptors (iRs) on myeloid immune populations including TAM (F4/80+), MDSC (Ly6C+) and N2-neutrophils (PMN) in MC38 carcinoma of difference sizes. Panels C-D depicts expression of CD47, a receptor for SIRPα, and PD-L1 on the same MC38 carcinoma of different sizes as in panel B. Panel E depicts representative IHC staining of samples of human cancers that display increased expression of ligands for myeloid iRs.

### Example 14

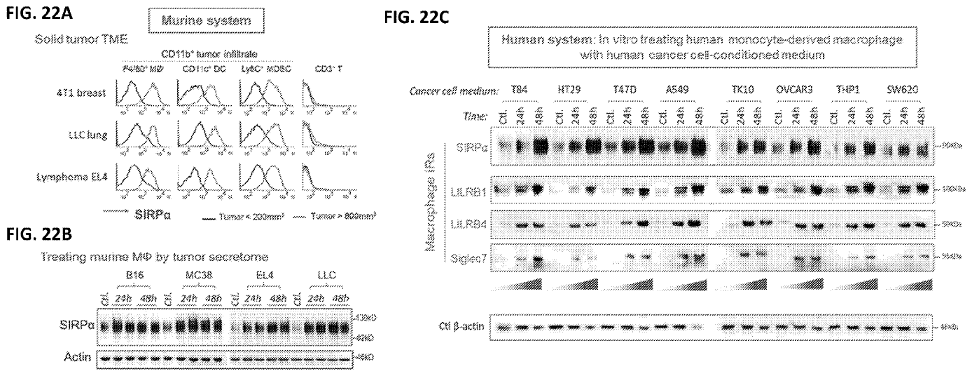

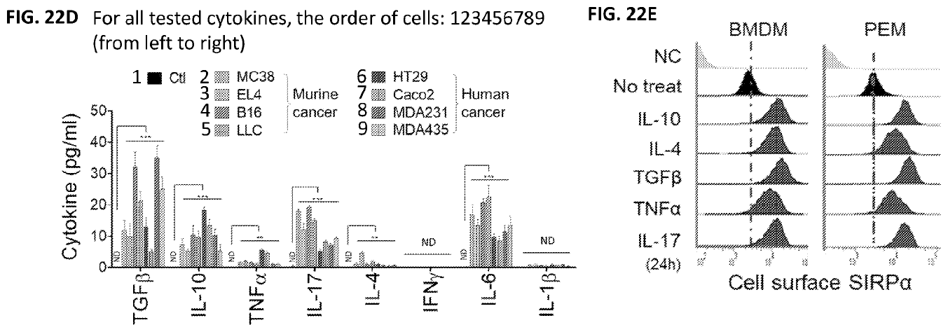

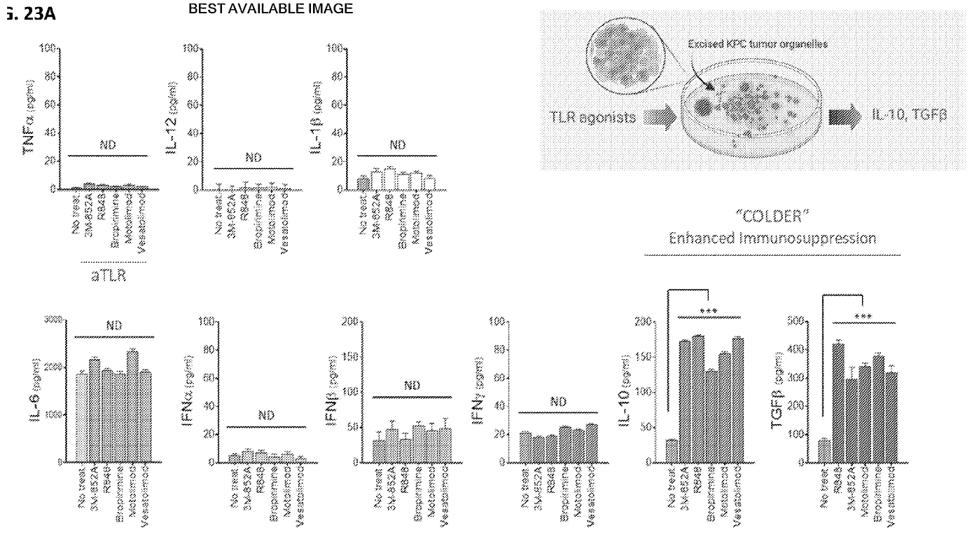

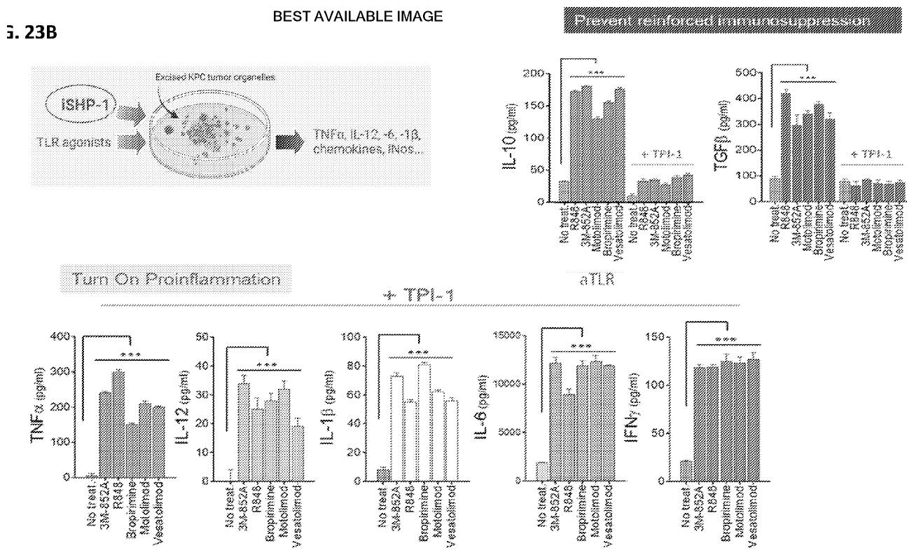

It was also found that cancer cells- and tumor TME-produced factors (secretome) induce increases in macrophage expression of iRs. See e.g., FIG. 22. Panel A shows increased expression of iRs on myeloid leukocytes in murine solid tumors including 4T1 breast cancer, LLC lung cancer and EL4 T cell lymphoma. Treating murine bone marrow-derived macrophages with murine cancer cells-conditioned medium induced increases in iR expression. Examples of cancer cells are B16 melanoma cells, MC38 colorectal carcinoma cells, EL4 T cell lymphoma cells and LLC lung cancer cells. SIRPα is an example of iR. See panel B of FIG. 22.

Treating human monocytes-derived macrophages with human cancer cells-conditioned medium induced increased expression of iRs. Examples of cancer cells shown are: colorectal cancer cells T84, HT29 and SW620T, breast cancer cell T47D, lung cancer A549, kidney cancer TK10, ovarian cancer OVCAR3, and monocytic leukemia THP1. Examples of iRs tested on human macrophages are: SIRPα, LILRB1, LILRB4 and Siglec7. See panel C of FIG. 22. Cytokines produced by murine and human cancer cells in culture were shown in panel D of FIG. 22. Treating macrophages with cancer cell cytokines induced increased iR expression (SIRPα as an example) on macrophages (bone marrow derived macrophages (BMDM) and peritoneal macrophages (PEM). See panel E of FIG. 22.

### Example 15

Inhibition of SHP-1 unleashes proinflammatory response in KPC tumor TME as shown in FIG. 23A-23B. KPC pancreatic tumor removed from mice were cut into small pieces and treated with 5 different TLR agonists (R848, 3M-852A, Motolimod, Bropirimine or Vesatolimod; 1 μg/ml each) without or with SHP-1 inhibitor TPI-1 (0.4 μM). After 18 h, cytokines secreted into the culture medium were assayed.

As shown in FIG. 23A, all five TLR agonists failed to trigger proinflammatory cytokine production but induced high level IL-10 and TGFβ, suggestive of enhanced immunosuppression in TME.

As shown in FIG. 23B, inhibition of SHP-1 (iSHP-1) enabled TLR agonists to drive proinflammatory response in tumor tissues. As shown, addition of SHP-1 inhibitor TPI-1 into A) induced high production of proinflammatory cytokine s while suppressing production of IL-10 and TGFβ.

### Example 16

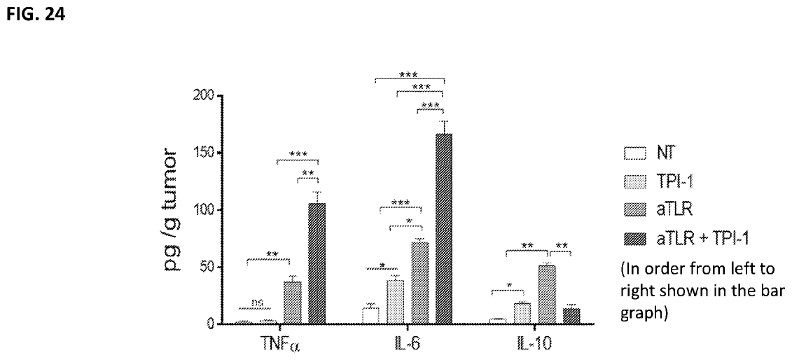

Treating MC38 tumor with TLR agonist (αTLR) plus SHP-1 inhibition led to proinflammatory polarization of TME. MC38 colorectal tumor excised from mice were cut into small pieces and treated with CpG+Poly(I:C)+R848 (each 0.4 ug/ml)±TPI-1 (0.4 μM). After 18 h, cytokines secreted into the culture medium were assayed. As shown in FIG. 24, αTLR plus TPI-1 induced a high level of proinflammatory response while suppressing IL-10 in the TME.

### Example 17

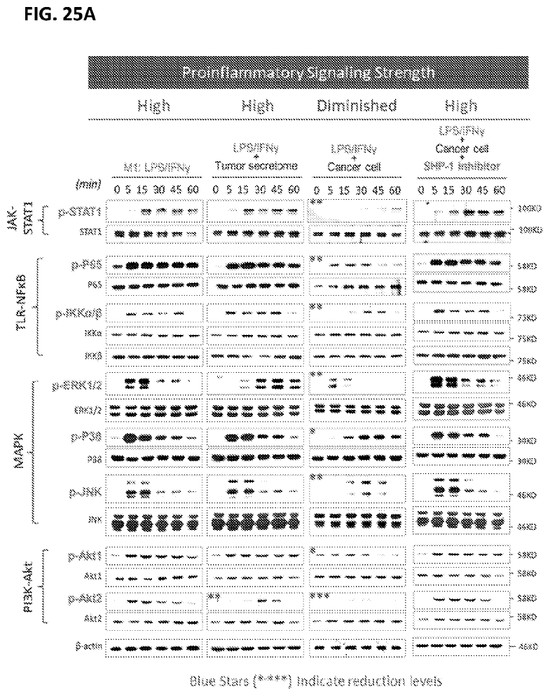

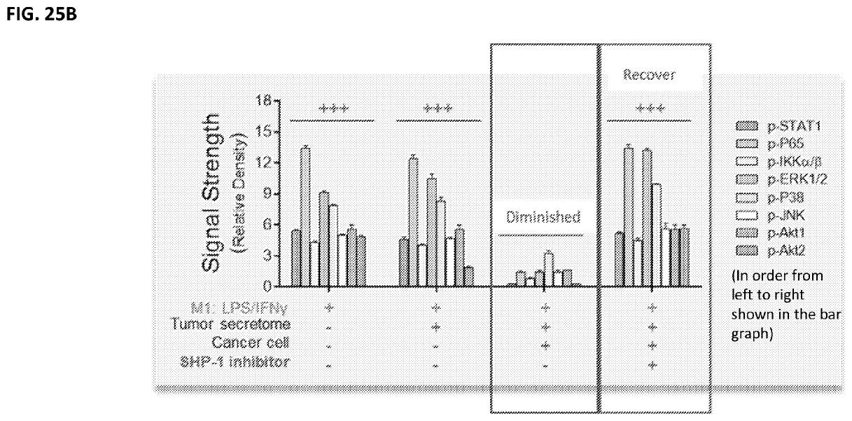

The iRs→SHP-1 inhibitory axis was studied. Activated SHP-1 by iRs dephosphorylates inflammatory stimuli-induced JAK-STAT, NFκB, MAPK, and PI3K-Akt activation pathways, quenching proinflammatory signaling and conferring therapeutic resistance. Inhibition of SHP-1 abolishes multi-axis iRs-mediated inhibitory regulation, unleashing macrophage proinflammatory polarization as shown in FIGS. 25A-25B.

### Example 18

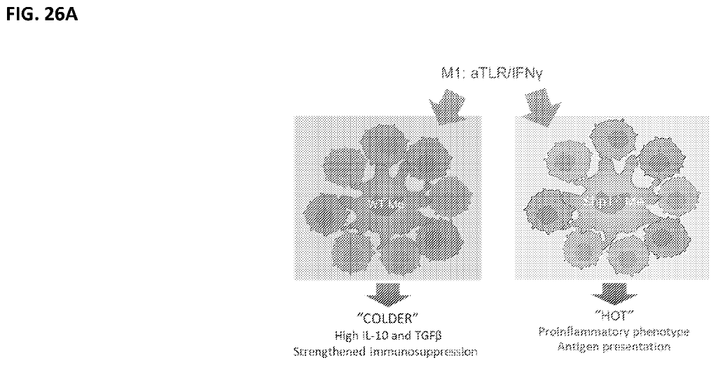

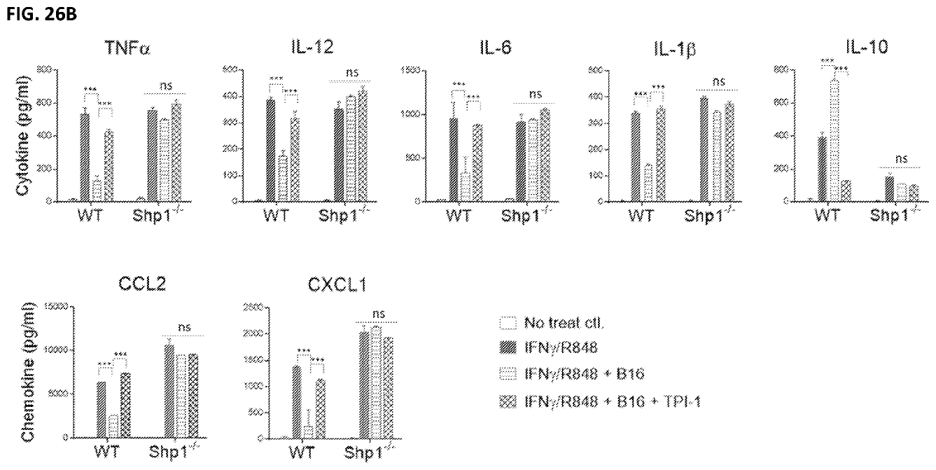

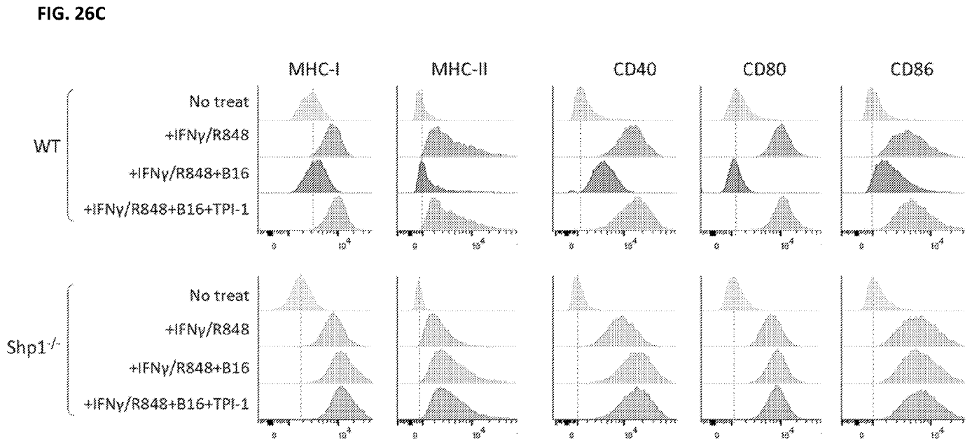

As shown in FIGS. 26B and 26C, Shp1^{-/-} macrophages resist cancer cells-imposed inhibition and unleash proinflammatory response under TLR and IFNγ stimulation. Murine bone marrow-derived macrophages (BMDM) prepared from WT, or homozygous Shp1^{-/-} mice were treated with TLR agonist (αTLR; R848, 1 μg/ml) plus IFNγ (40 ng/ml) in the presence of B16 melanoma cells. See FIG. 26A. After 16 h, cell culture medium were collected and assayed for cytokines. Results were shown in FIG. 26B. Macrophages were also collected and assayed for inflammatory phenotype and cell surface expression of antigen presentation machinery. Results were shown in FIG. 26C.

### Example 19

As shown in FIG. 27A-27C, cell surface blockade of iRs or ligands are alternative strategies to deplete the iRs→SHP-1 axis of inhibition.

Experimental setting: human macrophages co-cultured with cancer cells were stimulated with proinflammatory factors (e.g. TLR agonists, IFNγ, Sting activators, etc.) in the presence or absence of TPI-1 (inhibition of SHP-1, or iSHP-1), or mAbs that blockade Siglecs (aSiglec-7, -8 or -9), mAbs that blockade the LilRB-MHC interactions (αLILRB1, αLilRB2, αLIlRB3, αLilRB4, or a pan-HLA-A/B/C blockade Ab), or mAbs that blockade the CD47-SIRPα interaction (αCD47 or αSIRPα). To remove cancer cell surface sialic acid structures, which ligate Siglecs on macrophages, cancer cells were also treated with neuraminidase (50 mU/ml, 1 h) prior to use in experiments. See FIG. 27A.

Information of blockade antibodies used in experiments was shown in FIG. 27B. These antibodies were commercially available and were used at 2-10 μg/ml.

An example of data (macrophages with SW260 cancer cells). As shown in FIG. 27C, blockade of a single iR-ligand axis was inadequate to remove cancer cells-imposed immunosuppression on macrophages, whereas blockade multiple iR-ligand interactions or inhibition of SHP-1 downstream of all iRs in macrophages abrogated tumor cell inhibition, unleashing macrophages for proinflammatory response. Similar results were obtained when macrophages were co-cultured with other cancer cells such as OVCAR3, MDA231, TK10, HT29, etc.

### REFERENCES

1. Kang, X. L., J. Kim, M. Deng, S. John, H. Y. Chen, G. J. Wu, H. Phan, and C. C. Zhang. 2016. Inhibitory leukocyteimmunoglobulin-like receptors: Immune checkpoint proteins and tumor sustaining factors. Cell Cycle 15: 25-40.
2. Zarrin, A. A., and R. C. Monteiro. 2020. Editorial: The Role of Inhibitory Receptors in Inflammation and Cancer. Front Immunol 11.
3. Carosella, E. D., S. Gregori, and D. Tronik-Le Roux. 2021. HLA-G/LILRBs: A Cancer Immunotherapy Challenge. Trends Cancer 7: 389-392.
4. Yanagita, T., Y. Murata, D. Tanaka, S. I. Motegi, E. Arai, E. W. Daniwijaya, D. Hazama, K. Washio, Y. Saito, T. Kotani, H. Ohnishi, P. A. Oldenborg, N. V. Garcia, M. Miyasaka, O. Ishikawa, Y. Kanai, T. Komori, and T. Matozaki. 2017. Anti-SIRP alpha antibodies as a potential new tool for cancer immunotherapy. Jci Insight 2.
5. Zhang, W., Q. Huang, W. Xiao, Y. Zhao, J. Pi, H. Xu, H. Zhao, J. Xu, C. E. Evans, and H. Jin. 2020. Advances inAnti-Tumor Treatments Targeting the CD47/SIRPαlpha Axis. Front Immunol 11: 18.
6. Green, M. C., and L. D. Shultz. 1975. Motheaten, an immunodeficient mutant of the mouse. I. Genetics and pathology. J Hered 66: 250-258.
7. Shultz, L. D., and M. C. Green. 1976. Motheaten, an immunodeficient mutant of the mouse. II. Depressedimmune competence and elevated serum immunoglobulins. J Immunol 116: 936-943.
8. Kundu, S., K. Fan, M. L. Cao, D. J. Lindner, Z. Z. J. Zhao, E. Borden, and T. L. Yi. 2010. Novel SHP-1 Inhibitors Tyrosine Phosphatase Inhibitor-1 and Analogs with Preclinical Anti-Tumor Activities as Tolerated Oral Agents. J Immunol 184: 6529-6536.
9. Yuan, X., Y. Duan, Y. Xiao, K. Sun, Y. Qi, Y. Zhang, Z. Ahmed, D. Moiani, J. Yao, H. Li, L. Zhang, A. E. Yuzhalin, P. Li, C. Zhang, A. Badu-Nkansah, Y. Saito, X. Liu, W. L. Kuo, H. Ying, S. C. Sun, J. C. Chang, J. A. Tainer, and D. Yu. 2022. Vitamin E Enhances Cancer Immunotherapy by Reinvigorating Dendritic Cells via Targeting CheckpointSHP-1. Cancer Discov.
10. Arabaci, G., X. C. Guo, K. D. Beebe, K. M. Coggeshall, and D. Pei. 1999. alpha-Haloacetophenone derivatives asphotoreversible covalent inhibitors of protein tyrosine phosphatases. J Am Chem Soc 121: 5085-5086.
11. Yang, R. L., Q. Dong, H. B. Xu, X. H. Gao, Z. Y. Zhao, J. C. Qin, C. Chen, and D. Q. Luo. 2020. Identification of Phomoxanthone A and B as Protein Tyrosine Phosphatase Inhibitors. Acs Omega 5: 25927-25935.
12. Ben-Shmuel, A., B. Sabag, A. Puthenveetil, G. Biber, M. Levy, T. Jubany, F. Awwad, R. K. Roy, N. Joseph, O. Matalon, J. Kivelevitz, and M. Barda-Saad. 2022. Inhibition of SHP-1 activity by PKC-theta regulates NK cellactivation threshold and cytotoxicity. Elife 11.
13. Jones, M. L., J. D. Craik, J. M. Gibbins, and A. W. Poole. 2004. Regulation of SHP-1 tyrosine phosphatase inhuman platelets by serine phosphorylation at its C terminus. J Biol Chem 279: 40475-40483.
14. Shi, L., K. Kidder, Z. Bian, S. K. T. Chiang, C. Ouellette, and Y. Liu. 2021. SIRPαlpha sequesters SHP-2 to promote IL-4 and IL-13 signaling and the alternative activation of macrophages. Sci Signal 14: eabb3966.
15. Huang, Z., J. M. Coleman, Y. Su, M. Mann, J. Ryan, L. D. Shultz, and H. Huang. 2005. SHP-1 regulates STAT6phosphorylation and IL-4-mediated function in a cell type-specific manner. Cytokine 29: 118-124.
16. Johnson, D. J., L. I. Pao, S. Dhanji, K. Murakami, P. S. Ohashi, and B. G. Neel. 2013. SHP-1 regulates T cell homeostasis by limiting IL-4 signals. J Exp Med 210: 1419-1431.
17. Tao, B., W. Jin, J. Xu, Z. Liang, J. Yao, Y. Zhang, K. Wang, H. Cheng, X. Zhang, and Y. Ke. 2014. Myeloid-specific disruption of tyrosine phosphatase Shp2 promotes alternative activation of macrophages and predisposes mice to pulmonary fibrosis. J Immunol 193: 2801-2811.
18. Fisher, D. T., M. M. Appenheimer, and S. S. Evans. 2014. The two faces of IL-6 in the tumor microenvironment. Semin Immunol 26: 38-47.
19. Chonov, D. C., M. M. K. Ignatova, J. R. Ananiev, and M. V. Gulubova. 2019. IL-6 Activities in the Tumour Microenvironment. Part 1. Open Access Maced J Med Sci 7: 2391-2398.

1-2. (canceled)

3. A method of treating a cancer in an individual, comprising administering to the individual a) a SHP-1 inhibitor, and b) a pro-inflammatory agent, wherein the pro-inflammatory agent comprises an agent selected from the group consisting of a TLR agonist, a STING activator, a PAMP/DAMP activator, a chemotherapy, a pro-inflammatory cytokine, a cancer vaccine, an oncolytic virus, immune cells, a bacteria component, a sound treatment, a magnetic therapy, an electrical treatment, and an electrostatic treatment.

4. A method of treating a cancer in an individual, comprising administering to the individual a SHP-1 inhibitor, wherein the individual is under an inflammation reaction.

5. The method of claim 3, wherein the method further comprises administering the SHP-1 inhibitor to the individual intermittently.

6. The method of claim 3, wherein the method comprises systemically administering the SHP-1 inhibitor.

7. The method of claim 5, wherein the method comprises administering the SHP-1 inhibitor at an interval of no more than once every three days for at least twice.

8-9. (canceled)

10. The method of claim 1, wherein the SHP-1 inhibitor has a half-life of no more than about 5 days.

11-12. (canceled)

13. The method of claim 3, wherein the SHP-1 inhibitor is selected from the group consisting of TPI-1 or an analog or a derivative thereof, vitamin E derivative, phomoxanthone A (PXA), and a PKCθ activator.

14-15. (canceled)

16. The method of claim 3, wherein the method comprises administrating the SHP-1 inhibitor systemically or locally.

17. The method of claim 16, wherein the systemic administration of SHP-1 comprises oral administration, intravenous administration, subcutaneous administration, and/or intraperitoneal administration.

18. The method of claim 3, wherein the pro-inflammatory agent and the SHP-1 inhibitor are administered within 24 hours of each other.

19. The method of claim 3, wherein the method comprises intratumorally administering the pro-inflammatory agent and/or administering the pro-inflammatory agent to a site that is different from the site of the cancer to be treated.

20-21. (canceled)

22. The method of claim 3, wherein the pro-inflammatory agent comprises;
(1) a TLR agonist comprising CpG, polyL:C, and/or R848;
(2) a bacteria component comprising lipopolysaccharide (LPS);
(3) a STING activator comprising 2′3′ cGAMP;
(4) a chemotherapeutic agent comprising azathioprine (AZA);
(5) a proinflammatory cytokine selected from the group consisting of IL-1b, IL-18, IL-6, TNFα, and any combination thereof;
(6) a radiation therapy wherein the dose of the radiation therapy is insufficient to kill tumor cells; and/or
(7) a checkpoint inhibitor comprising an anti-PD-L1 antibody, an anti-PD-1 antibody, or an anti-CLTA4 antibody.

23-38. (canceled)

39. The method of claim 3, wherein the pro-inflammatory agent and the SHP-1 inhibitor are administered simultaneously or concurrently.

40. The method of claim 3, wherein the pro-inflammatory agent comprises immune cells.

41-52. (canceled)

53. The method of claim 1, further comprising administering to the individual an effective amount of an anti-TNFα antibody.

54. The method of claim 3, wherein the cancer is a solid tumor.

55. (canceled)

56. The method of claim 3, wherein the cancer is a late stage cancer.

57. The method of claim 1, wherein the cancer is resistant or refractory to a radiation therapy, a chemotherapeutic agent, and/or a checkpoint inhibitor.

58. The method of claim 1, wherein the individual is a human.

59. A composition comprising a SHP-1 inhibitor and a pro-inflammatory agent, wherein the pro-inflammatory agent comprises an agent selected from the group consisting of immune cells, a TLR agonist, a STING activator, an agent used in radiation therapy, a PAMP/DAMP activator, a checkpoint inhibitor, a pro-inflammatory cytokine, a chemotherapeutic agent, a bacteria component, a cancer vaccine, an oncolytic virus, and an agent used in sound treatment, a magnetic therapy, an electrical treatment or an electrostatic treatment.
